(19) United States
(12) Patent Application Publication
(10) Pub. No.: US 2008/0171064 A1
(43) Pub. Date: Jul. 17, 2008

(54) Live Attenuated Salmonella For Use as Vaccine

(75) Inventor: Tetsuo Mizuno, Queensland (AU)

Correspondence Address: DUANE MORRIS, LLP;IP DEPARTMENT, 30 SOUTH 17TH STREET, PHILADELPHIA, PA, 19103-4196, US

(73) Assignee: THE UNIVERSITY OF QUEENSLAND, ST. LUCIA (AU)

(21) Appl. No.: 10/589,065

(22) PCT Filed: Feb. 14, 2005
(86) PCT No.: PCT/AU2005/000187

§ 371 Date: Oct. 12, 2007

(30) Foreign Application Priority Data
Feb. 12, 2004 (AU) 2004900675
Feb. 12, 2004 (AU) 2004900679

(51) Int. Cl. A61K 39/02; A61K 39/112; C12N 1/20

(52) U.S. Cl. 424/235.1; 424/234.1; 424/258.1; 435/252.8

(57) ABSTRACT

The present invention relates to live attenuated Salmonella cultures for use as vaccines. The Salmonella cultures of the present invention have a substantially reduced capacity to grow and replicate in the presence of bile. The reduced capacity for growth is due to a metabolic-drift mutation induced by exposure to a combination of nalidixic acid and rifampicin for a time and under conditions sufficient to induce the mutation.

### BACKGROUND OF THE INVENTION

1. Field of the Invention

The present invention relates generally to a therapeutic agent. More particularly, the present invention provides a therapeutic agent in the form of a microorganism which has a substantially reduced capacity to grow and replicate due to microbiostatic agents present in, or introduced to, an environment within a subject to which the microorganism is administered, but which is capable of inducing a humoral and/or cell-mediated immune response to cell surface antigens or antigens secreted, or released, from the microbial cell. Antigens contemplated by the present invention include antigens naturally occurring on, or secreted from, the microbial cell as well as antigens produced through recombinant means such as antigens from other microorganisms, viruses, and parasites. Even more particularly, the therapeutic agent is a species of Salmonella or related organism. The therapeutic agent provided by the present invention is useful, inter alia, for the prophylaxis, amelioration or treatment of a range of diseases and conditions due to bacterial, viral, fungal and parasitic infections.

2. Description of the Prior Art

Reference to any prior art in this specification is not, and should not be taken as, an acknowledgment or any form of suggestion that this prior art forms part of the common general knowledge in any country.

Bibliographic details of references provided in this document are listed at the end of the specification.

Since their development, vaccines have successfully resulted in the eradication of major diseases such as smallpox and have lead to a dramatic reduction in other diseases such as poliomyelitis, hepatitis, measles and tetanus.

An immune response to a foreign antigen generally comprises two mechanisms. A humoral response, which is largely an antibody response directed to antigens present on pathogens in body fluids and a cell-mediated response which is largely a cell based response directed to pathogens which have infected host cells. Activation of the immune system leads to the creation of memory cells that can recognize and repel the same antigens if these reappear in the body.

Vaccines function by eliciting an immune response. However, vaccines vary in the kind and duration of immune protection they can provide. Those based on inactivated or “killed” antigens generally elicit a humoral response only. Such responses are ineffective against pathogens which infiltrate host cells and generally their protection wears off over time which necessitates “booster” vaccinations. Live attenuated vaccines, on the other hand, also have the capacity to elicit a cell-mediated immune response and generally the immunity they provide lasts for the life of the vaccinated subject.

One disease which has yet to be effectively controlled on a global scale is salmonellosis. Salmonellosis is a complex zoonotic disease in terms of its epidemiology, pathogenesis and control. It is a disease arising from infection by certain serotypes of Salmonella. In humans, salmonellosis may present clinically as a variety of conditions including gastroenteritis, enteric fever, bacteraemia and focal disease.

Although some advances have been made in strategies and techniques for its control in both humans and animals, salmonellosis still constitutes a major problem in both developed and developing countries (Gomez et al., World Health Statistics Quarterly 50:81-89, 1997; World Health Organization, Salmonellosis control: the role of animal and product hygiene pp 7-78, World Health Organization, Geneva, 1988).

Salmonella enterica subspecies enterica serotype Dublin (S. dublin) is one example of a microorganism which can cause salmonellosis. Salmonella dublin is a host-adapted bacterium which mainly colonizes cattle and calves. Major symptoms of S. dublin infection are enteritis and septicaemia in calves, enteritis in adult cattle and abortion in pregnant animals (Field, Veterinary Journal 104:251-266, 294-302, 323-339, 1948; Gibson, Veterinary Record 73:1284-1295, 1961; Hinton, British Veterinary Journal 130:556-562, 1974; Wray and Sojka, Journal of Dairy Research 44:383-425, 1977). One of the most important features of S. dublin infection is the development of a prolonged carrier state with resultant shedding of bacteria into the environment. The carrier state is unaffected by antimicrobial therapy and bacterial shedding may continue for several years and even throughout the life of the carrier (Vandergraff and Malmo, Australian Veterinary Journal 53:453-455, 1977; Wray, Veterinary Record 116:485-489, 1985; Wray, Irish Veterinary Journal 46:137-140, 1993). This organism, therefore, often becomes endemic to some regions.

Salmonella dublin infection in cattle has been reported in numerous countries around the world, particularly the United Kingdom (UK), Ireland, the United States of America (USA), Australia and many continental European nations (Gibson, Journal of Dairy Research 32:97-134, 1965; Wray and Sojka, 1977, Supra; Taylor et al., Journal of Infectious Diseases 146:322-327, 1982; Bruner, Cornell Veterinarian 75:93-96, 1985; Wray, 1985, Supra; Wray, 1993, Supra; Imberechts, Salmonella serotypes analysed at the VAR in 2000 pp 4-27, Veterinary and Agrochemical Research Centre, Brussels, Belgium, 2001; Murray et al., Australian Salmonella Reference Centre Annual Report 2000 p 4, Institute of Medical and Veterinary Science, Adelaide, Australia, 2001).

Whilst S. dublin rarely results in salmonellosis in human subjects, infections that do occur are generally more severe than with other Salmonella serovars, and the disease is often fatal (The Centres for Disease Control and Prevention, MMWR 43:1-7, 1994). In addition the latter report also states that the major cause of human S. dublin infection in the USA was the consumption of contaminated certified raw milk. Salmonella dublin is, therefore, an important food borne pathogen. Furthermore, increases in the use of antimicrobial agents in dairy and beef cattle over the last 50 years have caused the emergence of multiple antimicrobial agent resistant strains in some Salmonella serovars, such as Salmonella enterica subspecies enterica serotype Typhimurium (S. typhimurium) DT104. Preventing salmonellosis by vaccination should, therefore, reduce the risk of such strains emerging in the future, making the development of new improved Salmonella vaccines a priority.

Vaccines against S. dublin infection have been produced using both killed and live Salmonella. Killed vaccines are commercially available in some countries to prevent salmonellosis caused by S. dublin and S. typhimurium in calves and adult cattle. However, although the use of S. dublin killed vaccines is reasonably safe, the protection they offer is generally modest (House and Smith, USAHA Proceedings: evaluation of bovine Salmonella vaccines United States Animal Health Association, USA, 1997). Live S. dublin vaccines, on the other hand, confer better protection to vaccinated animals by inducing a greater cell-mediated immunity (Smith et al., American Journal of Veterinary Research 45:2231-2235, 1984). This is believed to be particularly important in the inactivation of facultative intracellular organisms such as Salmonella (Lindberg and Robertsson, Infection and Immunity 41:751-757, 1983). It is, however, difficult to produce prolonged cell-mediated immunity because this type of immunity is usually active for only a limited time span (Smith, 1984, Supra). Moreover, live Salmonella vaccines may be potentially pathogenic when administered to animals in poor health or to pregnant animals. For these and other reasons, use of S. dublin live vaccines is often restricted (World Health Organization, 1988, Supra).

In work leading up to the present invention, the inventors reasoned that since attenuated strains of salmonella can function as efficient tools for inducing immunity against salmonellosis, they may also act as potential carriers for the expression and delivery of heterologous antigens to the immune system. The present invention provides, therefore, a microorganism with the capability to function, inter alia, as a live attenuated vaccine that is both effective and safe to use.

### SUMMARY OF THE INVENTION

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Nucleotide and amino acid sequences are referred to by sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided at the end of the specification.

Abbreviations used herein are defined in Table 2.

The present invention provides a therapeutic agent. More particularly, the present invention provides, in one embodiment, a therapeutic agent comprising a microorganism which has a reduced capacity to grow and replicate in the presence of a microbiostatic substance present in, or introduced to, an environment within a subject into which said microorganism is administered wherein said microorganism is capable of inducing an immune response in said subject, which immune response is directed against an antigen on, or secreted by, the microorganism.

The preferred subjects to which the therapeutic agent of the present invention is administered are livestock species such as cattle, sheep and pigs as primates such as well as humans.

The present invention is predicated in part on the determination that microorganisms can be selected according to their inability to grow and replicate in the presence of a microbiostatic agent. This facilitates the selection of a substantially attenuated microorganism which can carry an antigen to the immune system of a host such that it can elicit an immune response. The immune response may be humoral and/or T-cell-mediated. In one embodiment, the humoral immune response is a mucosal immune response.

The ability to select for a substantially attenuated microorganism which can elicit an immune response in a host allows their use as a therapeutic agent in the treatment of disease. The therapeutic agent provided by the present invention is useful, inter alia, for the prophylaxis, amelioration or treatment of a range of diseases and conditions, due to bacterial, viral, fungal and parasitic infections.

Any microorganism may be used as the therapeutic agent of the present invention. Preferred microorganisms of the present invention are serotypes of Salmonella. Most preferably, the microorganisms of the present invention are of the S. dublin serotype. An immune response against S. dublin in cattle, sheep and pigs is useful in preventing infection and spontaneous abortions in those animals including enteritis and septicaemia.

The selection of a microorganism that is substantially incapable of growth and multiplication in the presence of a microbiostatic agent may be performed using any microbiostatic agent that will not harm the host. The preferred microbiostatic agent of the present invention is bile and the microorganism is selected by exposure to naladixic acid and rifampicin or their chemical or functional equivalents for a time and under conditions to induce a metabolic-drift mutation.

In yet another embodiment, the present invention provides a therapeutic agent comprising a modified microorganism which has a substantially reduced capacity to grow and replicate due to a microbiostatic agent present in, or introduced to, an environment within the host to which it migrates to following administration, wherein said modified microorganism comprises and/or expresses heterologous DNA sequences.

Still another aspect of the present invention contemplates a method of vaccinating a subject against a microorganism or an antigen produced by a microorganism said method comprising selecting a microorganism, exposing the microorganism to naladixic acid and rifampicin or their chemical or functional equivalents for a time and under conditions sufficient to induce a metabolic-drift mutation which renders the microorganism substantially unable to grow or replicate in the presence of a selected microbiostatic agent, and administering said mutated microorganism to the subject under conditions sufficient for the microorganism to migrate to an environment comprising the microbiostatic agent where it maintains itself for a time sufficient for an immune response to be induced to the microorganism or an antigen produced thereby.

TABLE 1
SUMMARY OF SEQUENCE IDENTIFIERS

| SEQUENCE ID NO: | DESCRIPTION |
|---|---|
| 1 | rpoB primer |
| 2 | rpoB primer |
| 3 | rpoB primer |
| 4 | rpoB primer |
| 5 | Nucleotide sequence of rpoB deletion in S. dublin |
| 6 | Nucleotide insertion in rpoB gene in S. dublin |

TABLE 2
ABBREVIATIONS

| ABBREVIATION | DESCRIPTION |
|---|---|
| AGRF | Australian Genome Research Facility |
| AR | Analytical regent |
| Arg | Arginine |
| Asp | Aspartic acid |
| BHI | Brain-heart infusion |
| BDNF | Brain-derived neurotrophic factor |
| bp | Base pair(s) |
| BS | Bile salt(s) |
| B.V. | Booster vaccination |
| cfu | Colony forming unit(s) |
| CLED | Cystine-Lactose-Electrolyte Deficient |
| CM | Cooked meat |
| CNTF | Cilliary-derived neurotrophic factor |
| Cys | Cysteine |
| DDW | Double distilled water |
| DI | Direct isolation |
| DNA | Deoxyribonucleic acid |
| dNTP | dinucleotide triphosphate |
| EGF | Epidermal growth factor |
| EI | Enrichment isolation |
| EPO | Erythropoitin |
| FGF | Fibroblast grwoth factor |
| G-CSF | Granulocyte colony stimulating factor |
| GH | Growth hormone |
| Gln | Glutamine |
| Glu | Glutamic acid |
| Gly | Glycine |
| GM-CSF | Granulocyte macrophage colony stimulating factor |
| h | Hours |
| HE | Hematoxylin and eosin |
| ID₅₀ | 50% infectious dose |
| IFN | Interferon |
| IL | Interleukin |
| Ile | Isoleucine |
| KDa | Kilo-dalton |
| LD | Lysin decarboxylase |
| LD₅₀ | 50% lethal dose |
| Leu | Leucine |
| LIF | Leukemia inhibitory factor |
| LP | Lamina propria |
| LPS | Lipopolysaccharide |
| LR | Laboratory reagent |
| M-CSF | Monocyte colony stimulating factor |
| MBC | Minimum bactericidal concentration |
| MCA | MacConkey agar No. 3 |
| MCP | Monocyte chemotactic protein |
| Met | Methionine |
| MHA | Mueller-Hinton agar |
| MIC | Minimum inhibitory concentration |
| min | Minute(s) |
| MIP | Macrophage inflammatory protein |
| MLN | Mesenteric lymph node |
| MN | Mononuclear |
| MSB | Mannitol selenite broth |
| NCCLS | The National Committee on Clinical Laboratory |
| | Standards |
| N-R | Resistant to nalidixic acid first and to rifampicin |
| | second |
| n | Number(s) |
| NGF | Nerve growth factor |
| NT | Neurotrophin |
| OD | Optical density |
| ONPG | O-Nitrophenyl-β-D-galactopyranoside |
| OSM | Oncostatin M |
| PBP | Platelet basic protein |
| PBS | Phosphate buffered saline |
| PBSF | Pre-B-cell growth stimulating factor |
| P.B.V. | Post booster vaccination |
| P.C. | Post challenge |
| PCR | Polymerase chain reaction |
| PDGF | Platelet-derived growth factor |
| PF | Platelet factor |
| Phe | Phenylalanine |
| P.I. | Post inoculation |
| PMN | Polymorphonuclear neutrophil granulocyte |
| PMSF | phenyl methyl-sulfonyl fluoride |
| pmol | Pico mole |
| Pro | Proline |
| P.V. | Post vaccination |
| QRDR | Quinolone-resistance determining region |
| R-N | Resistant to rifampicin first and to nalidixic acid |
| | second |
| RANTES | Regulated upon activation, normal T-cell expressed |
| RNA | Ribonucleic acid |
| RNAi | RNA interference |
| SBA | 10% Sheep blood agar |
| SCF | Sertoli cell factor |
| SD | Standard deviation |
| SDS | Sodium dodecyl sulfate |
| SDS-PAGE | Sodium dodecyl sulfate-polyacrylamide gel |
| | electrophoresis |
| sec | Second(s) |
| Ser | Serine |
| siRNA | Small interfering RNA |
| TEMED | N,N,N′,N′-tetramethylethylenediamine |
| TGF | Tranforming growth factor |
| Th cell | Helper T cell |
| TNF | Tumour necrosis factor |
| TPO | Thrombopoietin |
| TSB | Tryptone soya broth |
| TSI | Triple sugar iron |
| Tyr | Tyrosine |
| U | Unit |
| Val | Valine |
| VEGF | Vascular endothelial growth factor |
| XLD | Xylose lysine desoxycholate |
| ° C. | Degree Celsius |

### BRIEF DESCRIPTION OF THE FIGURES

FIG. 1 is a diagrammatical representation of the isolation and identification of Salmonella.

FIG. 2 is a graphical representation of growth curves for FD436 and nalidixic acid resistant strains N1 and N6 in TSB media determined by Bioscreen measurements of culture optical density (OD₄₂₀₋₅₈₀) taken every 10 min for 18 h.

FIG. 3 is a graphical representation of growth curves for FD436 and rifampicin resistant strain R3 in TSB media determined by Bioscreen measurements of culture optical density (OD₄₂₀₋₅₈₀) taken every 10 min for 18 h.

FIG. 4 is a graphical representation of growth curves for FD436 and N-R double antibiotic-resistant strains in TSB media determined by Bioscreen measurements of culture optical density (OD₄₂₀₋₅₈₀) taken every 10 min for 18 h.

FIG. 5 is a graphical representation of growth curves for FD436 and R-N double antibiotic-resistant strains in TSB media determined by Bioscreen measurements of culture optical density (OD₄₂₀₋₅₈₀) taken every 10 min for 18 h.

FIG. 6 is a photographic representation of agarose gel electrophoresis of amplified PCR products of S. dublin gyrA, the sizes of which were later confirmed by sequencing as 347 bp (arrow). Lanes: 1—1 k bp DNA marker; 2—S. dublin FD436; 3—N-RM4; 4—N-RM8; 5—N-RM9; 6—N-RM15; 7—N-RM20; 8—N-RM25; 9—N-RM27; 10—R-NM29.

FIG. 7 is a photographic representation of agarose gel electrophoresis of amplified PCR products of S. dublin rpoB, the sizes of which were later confirmed by sequencing as between 696 and 714 bp (arrow). Lanes: 1—1 k bp DNA marker; 2—S. dublin FD436; 3—N-RM4; 4—N-RM8; 5-N-RM9; 6—N-RM15; 7—N-RM20; 8—N-RM25; 9—N-RM27; 10—R-NM29.

FIG. 8 is a photographic representation of colonies of S. dublin wild strain FD436 (A) and mutant N-RM25 (B) growing on SBA plate after aerobic incubation at 37° C. for 24 hours. FD436 produced colonies that were white-grey, opaque, circular and convex, and that had an entire margin and a smooth surface. Compared with this, colonies of N-RM25 were smaller, translucent and slightly raised, and had a matte surface.

FIG. 9 is a photographic representation of lipopolysaccharide profiles of wild strain FD436 and metabolic-drift mutants determined by SDS-PAGE and ammoniacal silver staining (molecular weights are based on the migration of the protein marker). Lanes: 1-wild strain FD436; 2—mutant N-RM4; 3—N-RM8; 4—N-RM9; 5—N-RM15; 6—N-RM20; 7—N-RM25; 8—N-RM27; 9—R-NM29. Red arrows indicate bands.

FIG. 10 is a photographic representation of whole cell protein profiles of wild strain FD436 and metabolic-drift mutants determined by SDS-PAGE and Coomassie blue staining. Lanes: 1—broad range molecular weight standards; 2—wild strain FD436; 3—mutant N-RM4; 4—N-RM8; 5—N-RM9; 6—N-RM15; 7—N-RM20; 8—N-RM25; 9—N-RM27; 10—R-NM29. Black arrow band of approximately 53 kDa; Red arrow=band of approximately 24 kDa.

FIG. 11 is a graphical representation of growth curves of S. dublin wild strain FD436 and metabolic-drift mutants N-RM4 and 25 and R-NM29 in TSB containing graded concentrations of bile salts No. 3 determined by Bioscreen measurements of culture optical density (OD₄₂₀₋₅₈₀) taken every 10 min for 20 h.

FIG. 12 is a graphical representation of enumeration of Salmonella in liver plus spleen of mice 0-24 days following intraperitoneal inoculation with FD436, N-RM4, N-RM25 or R-NM29.

FIG. 13 is a photographic representation of a mouse euthanased on Day 7 following challenge with S. dublin metabolic-drift mutant N-RM4 (Group 2). Prominent splenomegaly (blue arrow) and grey-white foci (black arrows) scattered across the entire surface of liver (A) and an enlargement (B) of part of the liver in (A) showing scattered foci (arrows).

FIG. 14 is a photographic representation of a mouse euthanased on Day 12 following challenge with S. dublin metabolic-drift mutant N-RM25 (Group 3). No gross lesions are evident.

FIG. 15 is a photographic representation of acute focal hepatitis. (A) Mouse euthanased on Day 3 following challenge with S. dublin wild strain FD436 (Group 1). Prominent PMN infiltration and a suppurative focus (arrow). ×200, H&E stain. (B) Coagulative necrosis (arrow). ×400, H&E stain.

FIG. 16 is a photographic representation of penetration by Salmonella of liver of mice euthanased on Day 5 following challenge. Organisms were observed in Kupffer cells (black arrows) and PMNs (red arrow). (A) mouse given S. dublin wild strain FD436 (Group 1) and (B) mouse given S. dublin metabolic-drift mutant N-RM4 (Group 2). ×1000, Giemsa stain.

FIG. 17 is a photographic representation of periportal inflammatory infiltrate in liver. Mouse euthanased on Day 3 (A) and Day 24 (B) following challenge with S. dublin metabolic-drift mutant N-RM25 (Group 3). Note infiltrating cells are PMNs, lymphocytes and macrophages (A), and lymphocytes and plasma cells (B). ×200, H&E stain.

FIG. 18 is a photographic representation of clustered PMNs (arrow) in splenic sinus areas. Mouse euthanased on Day 3 following challenge with S. dublin metabolic-drift mutant N-RM4 (Group 2). ×400, H&E stain.

FIG. 19 is a photographic representation of the spleen of mouse euthanased on Day 24 following challenge with S. dublin metabolic-drift mutant N-RM25 (Group 3). Although there is moderate lymphoid hyperplasia, inflammation is not evident. ×200, H&E stain.

FIG. 20 is a photographic representation of the gall bladder from negative control mouse (Group 5). BB=brush border, EP=epithelium, LP=lamina propria, SM=submucosa, MW=muscular wall and S=serosa. ×400, H&E stain.

FIG. 21 is a photographic representation of cholecystitis. Mouse euthanased on Day 5 following challenge with S. dublin wild strain FD436 (Group 1). Epithelial hyperplasia and oedema of lamina propria are evident. Infiltration of lamina propria by MN cells and a small number of PMNs is also observed. ×400, H&E stain.

FIG. 22 is a photographic representation of penetration of gall bladder by Salmonella. Mouse euthanased on Day 5 following challenge with S. dublin wild strain FD436 (Group 1). The organisms are in the lumen (A) and epithelial cells (B—red arrows). Inflammatory infiltration by PMNs is evident. ×1000, Giemsa stain.

FIG. 23 is a photographic representation of severe acute cholecystitis. Mouse euthanased on Day 7 following challenge with S. dublin metabolic-drift mutant N-RM4 (Group 2). Epithelial hyperplasia, and oedema, haemorrhage and fibroplasia in lamina propria are prominent. Predominant infiltrating cells are PMNs. Subserosal oedema, fibroplasia and inflammatory infiltrate are also evident (A). ×200, H&E stain. Intraluminal inflammatory exudate of PMNs and MN cells associated with acute cholecystitis in the same mouse (B). ×400, H&E stain.

FIG. 24 is a photographic representation of penetration of gall bladder by Salmonella. Mouse euthanased on Day 7 following challenge with S. dublin metabolic-drift mutant N-RM4 (Group 2). (A) numerous organisms are present in the lumen and adhering to brush border (red arrows). (B) organisms are adhering to epithelial cells (red arrows). Some organisms are in epithelial cells (white arrow). (C) organisms are in epithelial cells (black arrows). Infiltration by PMNs is prominent. ×1000, Giemsa stain.

FIG. 25 is a photographic representation of the gall bladder of mouse euthanased on Day 5 following challenge with S. dublin metabolic-drift mutant N-RM25 (Group 3). (A) mild epithelial hyperplasia, congestion of lamina propria, margination in vessels (arrows) and mild infiltration suggesting early stage inflammation. ×400, H&E stain. (B) Salmonella are present in gall bladder lumen in low numbers (arrows). ×1000, Giemsa stain.

FIG. 26 is a photographic representation of the gall bladder of mouse euthanased on Day 24 following challenge with S. dublin metabolic-drift mutant N-RM25 (Group 3). Inflammation is not evident except very mild oedema of lamina propria and subserosa. ×400, H&E stain.

FIG. 27 is a graphical representation of the number of Salmonella in liver and spleen of mice (n=5/group) dosed using single and double vaccination methods with different doses of N-RM25 and challenged with wild strain FD436.

FIG. 28 is a graphical representation of the number of Salmonella in liver and spleen of mice vaccinated with N-RM4 or R-NM29 and challenged with wild strain FD436.

FIG. 29 is a graphical representation showing change in weight of calves between pre (Day-1)- and post (Day-16)-vaccination.

FIG. 30 is a graphical representation showing fibrinogen in peripheral blood of calves in intramuscular vaccine trials.

FIG. 31 is a graphical representation showing the number of white blood cells in peripheral blood of calves in intramuscular vaccine trials.

FIG. 32 is a graphical representation showing serum immunoglobulin G (as analysed by ELISA) against Salmonella dublin wild strain FD436 induced by N-RM25 vaccine administered via the intramuscular route.

FIG. 33 is a graphical representation showing serum immunoglobulin G (as analysed by ELISA) against Salmonella typhimurium wild strain DH436 induced by N-RM25 vaccine administered via the intramuscular route.

FIG. 34 is a graphical representation showing serum immunoglobulin A (as analysed by ELISA) against Salmonella dublin wild strain FD436 induced by N-RM25 vaccine administered via the intramuscular route.

FIG. 35 is a graphical representation showing serum immunoglobulin A (as analysed by ELISA) against Salmonella typhimurium wild strain DH436 induced by N-RM25 vaccine administered via the intramuscular route.

### DETAILED DESCRIPTION OF THE PREFERRED EMBODIMENTS

The present invention contemplates a therapeutic agent in the form of a microorganism which has a substantially reduced capacity to grow and replicate due to microbiostatic agents present in, or introduced to, an environment within the host to which the microorganism migrates following administration. The reduced capacity for growth is due to a metabolic-drift mutation following exposure of the microorganism to naladixic acid and rifampicin or their chemical or functional equivalents. This facilitates the selection of a substantially attenuated microorganism which can carry an antigen to the immune system of a host such that it can elicit an immune response. The therapeutic agent provided by the present invention is useful, inter alia, for the prophylaxis of a range of diseases or conditions, and in particular, diseases or conditions which can be treated, or symptoms ameliorated by, the elicitation of an immune response. Such diseases and conditions include, inter alia, infection by bacteria, viruses, fungi and parasites.

It is to be understood that unless otherwise indicated, the subject invention is not limited to specific therapeutic components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise. Thus, for example, reference to a “therapeutic agent” includes a single therapeutic agent, as well as two or more therapeutic agents; reference to a “microorganism” includes a single microorganism, as well as two or more microorganisms; and so forth.

In one embodiment the present invention provides a therapeutic agent comprising a microorganism which has a reduced capacity to grow and replicate in the presence of a microbiostatic substance present in, or introduced to, an environment within a subject to which said microorganism migrates following administration wherein said microorganism is capable of inducing an immune response in said subject, which immune response is directed against an antigen on, or secreted by, the microorganism.

The term “subject” as used herein refers to humans and non-human primates (e.g. gorilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer) and any other organisms who can benefit from the therapeutic agent of the present invention. There is no limitation on the type of animal that could benefit from the presently described therapeutic agents. The most preferred subjects of the present invention are livestock animals and humans. A subject regardless of whether it is a human or non-human may be referred to as a patient, subject, individual, animal, host or recipient.

Reference herein to “microorganism” means any prokaryotic organism (e.g. bacteria) or lower eukaryotic organism (e.g. algae, fungi, protozoa). The preferred microorganisms of the present invention are members of the Enterobacteriaceae group of bacteria. The Enterobacteriaceae group comprises, but is not limited to, Enterobacter (e.g. Enterobacter aerogenes, Enterobacter amnigenus, Enterobacter asburiae, Enterobacter cancerogenus, Enterobacter cloacae, Enterobacter cowanii, Enterobacter dissolvens, Enterobacter gergoviae, Enterobacter hormaechei, Enterobacter intermedius, Enterobacter kobei, Enterobacter nimipressuralis, Enterobacter pyrinus, Enterobacter sakazakii, Enterobacter sp., Enterobacter sp. ‘MS 412’, Enterobacter sp. 16-31, Enterobacter sp. 2002-2301161, Enterobacter sp. 22, Enterobacter sp. 253a, Enterobacter sp. 3-45, Enterobacter sp. 76996, Enterobacter sp. B2/69, Enterobacter sp. B24a, Enterobacter sp. B24b, Enterobacter sp. B41, Enterobacter sp. B509, Enterobacter sp. B5R5, Enterobacter sp. B901-2, Enterobacter sp. B96, Enterobacter sp. C1T5, Enterobacter sp. CC1, Enterobacter sp. CH2-4, Enterobacter sp. dtb33, Enterobacter sp. DW143a1, Enterobacter sp. DW56, Enterobacter sp. EK3.1, Enterobacter sp. EK4, Enterobacter sp. Fma, Enterobacter sp. IMD1260, Enterobacter sp. isolate #3, Enterobacter sp. Lgg10.16, Enterobacter sp. Lgg10.2, Enterobacter sp. Lgg5.6, Enterobacter sp. NAB4, Enterobacter sp. NAB5, Enterobacter sp. PD12, Enterobacter sp. RFL1396, Enterobacter sp. S23, Enterobacter sp. S4-27, Enterobacter sp. TUT1014), Erwinia (e.g. Candidatus Erwinia dacicola, Erwinia amylovora, Erwinia aphidicola, Erwinia billingiae, Erwinia chrysantum, Erwinia mallotivora, Erwinia papayae, Erwinia persicina, Erwinia psidii, Erwinia pyrifoliae, Erwinia rhapontici, Erwinia tracheiphila, Erwinia sp., Erwinia sp. 1, Erwinia sp. 2, Erwinia sp. 3, Erwinia sp. 4, Erwinia sp. 5, Erwinia sp. 6, Erwinia sp. DW1, Erwinia sp. Ejp 556, Erwinia sp. Ejp557, Erwinia sp. Ejp617, Erwinia sp. Lgg10.4, Erwinia sp. Lgg20.9, Erwinia sp. MB31, Erwinia sp. P18G1186, Erwinia sp. SK-30-7, Erwinia sp. TzT-JG-2-8), Escherichia (e.g. Escherichia albertii, Escherichia blattae, Escherichia coli, Escherichia coli 042, Escherichia coli B, Escherichia coli CFT073, Escherichia coli E2348, Escherichia coli K12, Escherichia coli O111:H-, Escherichia coli O127:H6, Escherichia coli O157:H-, Escherichia coli O157:H7, Escherichia coli O157:H7 EDL933, Escherichia coli O6, Escherichia fergusonii, Escherichia hermannii, Escherichia senegalensis, Escherichia vulneris, Escherichia sp. 253b, Escherichia sp. Souza-207, Escherichia sp. Souza-273, Escherichia sp. Souza-57), Klebsiella (e.g. Klebsiella aerogenes, Klebsiella granulomatis, Klebsiella milletis, Klebsiella oxytoca, Klebsiella cf. planticola B43, Klebsiella pneumoniae, Klebsiella pneumoniae subsp. ozaenae, Klebsiella pneumoniae subsp. pneumoniae, Klebsiella pneumoniae subsp. rhinoscleromatis, Klebsiella senegalensis, Klebsiella singaporensis, Klebsiella variicola, Klebsiella sp., Klebsiella sp. 141, Klebsiella sp. 6613, Klebsiella sp. 6A2, Klebsiella sp. 708, Klebsiella sp. 910, Klebsiella sp. A5054, Klebsiella sp. ABR11, Klebsiella sp. B2-1, Klebsiella sp. CC-88028, Klebsiella sp. CC-88037, Klebsiella sp. CC-88168, Klebsiella sp. Cd-1, Klebsiella sp. Ck-1, Klebsiella sp. DW40, Klebsiella sp. ES14.2, Klebsiella sp. HL1, Klebsiella sp. K2-1, Klebsiella sp. KCL-2, Klebsiella sp. KCL2, Klebsiella sp. KG1, Klebsiella sp. KGA, Klebsiella sp. LS13-39, Klebsiella sp. LX3, Klebsiella sp. M4112, Klebsiella sp. Ni3, Klebsiella sp. P2, Klebsiella sp. P3, Klebsiella sp. P4, Klebsiella sp. PN2, Klebsiella sp. rennanqilfy18, Klebsiella sp. RS1, Klebsiella sp. TK44, Klebsiella sp. VI), Proteus (e.g. Proteus inconstans, Proteus mirabilis, Proteus penneri, Proteus vulgaris, Proteus sp. LE138), Salmonella (e.g. Salmonella bongori, Salmonella bongori 12149, Salmonella bongori 66:z41:-, Salmonella choleraesuis, Salmonella choleraesuis subsp. arizonae, Salmonella choleraesuis subsp. choleraesuis, Salmonella choleraesuis subsp. diarizonae, Salmonella choleraesuis subsp. houtenae, Salmonella choleraesuis subsp. indica, Salmonella choleraesuis subsp. salamae, Salmonella dublin, Salmonella enterica, Salmonella enterica subsp. arizonae, Salmonella enterica subsp. diarizonae, Salmonella enterica IIIb 50:k:z, Salmonella enterica subsp. enterica, Salmonella enterica subsp. enterica serovar 1,4,[5],12,:i:1,2, Salmonella enterica subsp. enterica serovar Abony, Salmonella enterica subsp. enterica serovar Abortusequi, Salmonella enterica subsp. enterica serovar Abortusovis, Salmonella enterica subsp. enterica serovar Adelaide, Salmonella enterica subsp. enterica serovar Agona, Salmonella enterica subsp. enterica serovar Albany, Salmonella enterica subsp. enterica serovar Anatum, Salmonella enterica subsp. enterica serovar Austin, Salmonella enterica subsp. enterica serovar Azteca, Salmonella enterica subsp. enterica serovar Banana, Salmonella enterica subsp. enterica serovar Bareilly, Salmonella enterica subsp. enterica serovar Berta, Salmonella enterica subsp. enterica serovar Blockley, Salmonella enterica subsp. enterica serovar Borreze, Salmonella enterica subsp. enterica serovar Bovis-morbificans, Salmonella enterica subsp. enterica serovar Braenderup, Salmonella enterica subsp. enterica serovar Brandenburg, Salmonella enterica subsp. enterica serovar Bredeney, Salmonella enterica subsp. enterica serovar Budapest, Salmonella enterica subsp. enterica serovar Bury, Salmonella enterica subsp. enterica serovar California, Salmonella enterica subsp. enterica serovar Chester, Salmonella enterica subsp. enterica serovar Chingola, Salmonella enterica subsp. enterica serovar Choleraesuis, Salmonella enterica subsp. enterica serovar Cubana, Salmonella enterica subsp. enterica serovar Derby, Salmonella enterica subsp. enterica serovar Dublin, Salmonella enterica subsp. enterica serovar Enteritidis, Salmonella enterica subsp. enterica serovar Essen, Salmonella enterica subsp. enterica serovar Gallinarum, Salmonella enterica subsp. enterica serovar Gallinarum/pullorum, Salmonella enterica subsp. enterica serovar Give, Salmonella enterica subsp. enterica serovar Hadar, Salmonella enterica subsp. enterica serovar Haifa, Salmonella enterica subsp. enterica serovar Havana, Salmonella enterica subsp. enterica serovar Heidelberg, Salmonella enterica subsp. enterica serovar Infantis, Salmonella enterica subsp. enterica serovar Java, Salmonella enterica subsp. enterica serovar Kentucky, Salmonella enterica subsp. enterica serovar Kottbus, Salmonella enterica subsp. enterica serovar Liverpool, Salmonella enterica subsp. enterica serovar London, Salmonella enterica subsp. enterica serovar Maregrosso, Salmonella enterica subsp. enterica serovar Marrtens, Salmonella enterica subsp. enterica serovar Matopeni, Salmonella enterica subsp. enterica serovar Mbandaka, Salmonella enterica subsp. enterica serovar Mikawasima, Salmonella enterica subsp. enterica serovar Minnesota, Salmonella enterica subsp. enterica serovar Monschaui, Salmonella enterica subsp. enterica serovar Montevideo, Salmonella enterica subsp. enterica serovar Moscow, Salmonella enterica subsp. enterica serovar Muenchen, Salmonella enterica subsp. enterica serovar Muenster, Salmonella enterica subsp. enterica serovar Naestved, Salmonella enterica subsp. enterica serovar Newmexico, Salmonella enterica subsp. enterica serovar Newport, Salmonella enterica subsp. enterica serovar Ohio, Salmonella enterica subsp. enterica serovar Oranienburg, Salmonella enterica subsp. enterica serovar Ordonez, Salmonella enterica subsp. enterica serovar Othmarschen, Salmonella enterica subsp. enterica serovar Panama, Salmonella enterica subsp. enterica serovar Paratyphi B, Salmonella enterica subsp. enterica serovar Paratyphi C, Salmonella enterica subsp. enterica serovar Pensacola, Salmonella enterica subsp. enterica serovar Potsdam, Salmonella enterica subsp. enterica serovar Pullorum, Salmonella enterica subsp. enterica serovar Rachaburi, Salmonella enterica subsp. enterica serovar Reading, Salmonella enterica subsp. enterica serovar Rostock, Salmonella enterica subsp. enterica serovar Rubislaw, Salmonella enterica subsp. enterica serovar Saintpaul, Salmonella enterica subsp. enterica serovar Schleissheim, Salmonella enterica subsp. enterica serovar Senftenberg, Salmonella enterica subsp. enterica serovar Setubal, Salmonella enterica subsp. enterica serovar Shomron, Salmonella enterica subsp. enterica serovar Simsbury, Salmonella enterica subsp. enterica serovar Sloterdijk, Salmonella enterica subsp. enterica serovar Sofia, Salmonella enterica subsp. enterica serovar Stanley, Salmonella enterica subsp. enterica serovar Tejas, Salmonella enterica subsp. enterica serovar Tennessee, Salmonella enterica subsp. enterica serovar Tennyson, Salmonella enterica subsp. enterica serovar Texas, Salmonella enterica subsp. enterica serovar Thompson, Salmonella enterica subsp. enterica serovar Toulon, Salmonella enterica subsp. enterica serovar Travis, Salmonella enterica subsp. enterica serovar Typhi, Salmonella enterica subsp. enterica serovar Typhimurium, Salmonella enterica subsp. enterica serovar Typhisuis, Salmonella enterica subsp. enterica serovar Vellore, Salmonella enterica subsp. enterica serovar Virchow, Salmonella enterica subsp. enterica serovar Virginia, Salmonella enterica subsp. enterica serovar Waycross, Salmonella enterica subsp. enterica serovar Weltevreden, Salmonella enterica subsp. enterica serovar Wien, Salmonella enterica subsp. houtenae, Salmonella enterica IV 43:z4,z23:-, Salmonella enterica subsp. houtenae serovar Houten, Salmonella enterica subsp. indica, Salmonella enterica VI 1,6,14,25:a:e,n,x, Salmonella enterica subsp. salamae, Salmonella enterica subsp. salamae serovar 4,12,27:i:z35, Salmonella enterica subsp. salamae serovar 4,12:z:1,7, Salmonella enterica subsp. VII, Salmonella enteritidis, Salmonella paratyphi, Salmonella typhi, Salmonella typhimurium, Salmonella typhimurium D104, Salmonella typhimurium LT2, Salmonella typhimurium SL1344, Salmonella typhimurium TR7095, Salmonella sp., Salmonella sp. 4182, Salmonella sp. AHL 6, Salmonella sp. S126, Salmonella sp. S14, Salmonella sp. S191, Salmonella sp. TC67), Shigella (e.g. Shigella boydii, Shigella dysenteriae, Shigella dysenteriae M131649, Shigella flexneri, Shigella flexneri 2a, Shigella sonnei, Shigella sonnei 53G, Shigella sp.) and Yersinia (e.g. Yersinia aldovae, Yersinia bercovieri, Yersinia bercovieri (type 0:58,16), Yersinia enterocolitica, Yersinia enterocolitica (type 0:1), Yersinia enterocolitica (type 0:13,18), Yersinia enterocolitica (type 0:13a,13b), Yersinia enterocolitica (type 0:15), Yersinia enterocolitica (type 0:2), Yersinia enterocolitica (type 0:20), Yersinia enterocolitica (type 0:21), Yersinia enterocolitica (type 0:3), Yersinia enterocolitica (type 0:34), Yersinia enterocolitica (type 0:4,32), Yersinia enterocolitica (type 0:5,27), Yersinia enterocolitica (type 0:8), Yersinia enterocolitica (type 0:9), Yersinia frederiksenii, Yersinia intermedia, Yersinia kristensenii, Yersinia mollaretii, Yersinia pestis, Yersinia pestis C092, Yersinia pestis KIM, Yersinia pseudotuberculosis, Yersinia pseudotuberculosis (type O:1b), Yersinia rohdei, Yersinia ruckeri, Yersinia sp.).

Even more preferably, the microorganisms of the present invention are serotypes of Salmonella.

Accordingly, another embodiment of the present invention provides a therapeutic agent comprising a serotype of Salmonella which carries a metabolic-drift mutation and has a substantially reduced capacity to grow and replicate due to a microbiostatic agent present in, or introduced to, an environment within the host to which it migrates to following administration, wherein said serotype of Salmonella is capable of eliciting an immune response in a subject, which immune response is directed against an antigen produced by said Salmonella.

Preferably, the microorganism of the present invention functions as a “carrier”. Reference herein to a “carrier” means a microorganism which can carry an antigen (either naturally occurring or heterologous) such that it can elicit a desired biological response in a subject. Preferably the desired biological response is a humoral and/or cell-mediated immune response. Even more preferably, the biological response of the present invention results in the prevention, amelioration or treatment of symptoms associated with a disease or condition such as, but not limited to, salmonellosis.

Most preferably, the microorganism of the present invention is Salmonella dublin (S. dublin).

Accordingly another embodiment of the present invention provides a therapeutic agent comprising S. dublin which carries a metabolic-drift mutation and has a substantially reduced capacity to grow and replicate due to a microbiostatic agent present in, or introduced to, an environment within the host to which it migrates to following administration, wherein said S. dublin is capable of eliciting an immune response in a subject, which immune response is directed against an antigen produced by said S. dublin.

Reference herein to “S. dublin” also includes all S. dublin variants, such as those S. dublin which are genetically modified and S. dublin related serovars (i.e. other Salmonella species or sub-species with related antigenic properties).

The microorganisms contemplated by the present invention are substantially attenuated. Reference herein to “attenuated” means a microorganism that does not induce an infection or symptoms of an infection in a host compared to a non-attenuated organism present in the same amount yet is still capable of inducing an immune response.

One method of attenuation used in accordance with the present invention is genetic attenuation. Reference herein to “genetic attenuation” means attenuating a microorganism through modification of the microorganism genome using sense, antisense, RNAi, si-RNA or mutation technology. A preferred method of modifying the genome of the microorganism of the present invention is mutation. This involves exposing nucleic acids which comprise the genome of a microorganism to mutagens such as chemical and radiation mutagens to induce single or multiple nucleotide substitutions, additions, deletions and/or insertions into genes or genetic regions required for, or which, facilitate pathogenesis in or on a host.

In a particularly preferred embodiment of the present invention, the mutation of a microorganism genome is achieved by exposing the microorganism to high concentrations of nalidixic acid and rifampicin. This results in the induction of one or more metabolic drift mutations in the genome of the microorganism. The present invention extends to a single or a multiplicity of metabolic drift mutations such as 1, 2, 3, 4, 5, 6, 7, 8, 9 or 10 mutations.

The substantially attenuated microorganism of the present invention preferably has a substantially reduced capacity to grow and replicate due to a microbiostatic agent present in, or introduced to, an environment to which it is administered.

Reference herein to “substantially reduced capacity to grow and replicate” means that the microorganism is capable of survival and eliciting an immune response without growing and generating substantial numbers of progeny. This may be achieved by any means well known to those in the art. In a preferred embodiment it is achieved by a microbiostatic agent. Reference herein to a “microbiostatic agent” means an agent that will render a microorganism substantially incapable of growth and multiplication but which is not microbicidal at the concentration present. The term “microbiostatic agent” should also be taken to include a reference to an “inhibitory agent” and as such the terms may be used interchangably.

The microorganisms of the present invention are therefore selected according to their inability to grow and multiply in the presence of a microbiostatic agent. The preferred microorganisms of the present invention have a substantially reduced capacity to grow and replicate in the presence of a microbiostatic agent.

Although the microbiostatic agent of the present invention may also comprise any agent which has microbiostatic properties, in a preferred embodiment, the microbiostatic agent of the present invention is naturally present in the environment to which the microorganism of the present invention is administered. Most preferably, the microbiostatic agent of the present invention is bile.

Accordingly, another embodiment of the present invention contemplates a therapeutic agent comprising a Salmonella sp. which carries a metabolic-drift mutation resulting in a reduced capacity to grow and replicate in the presence of bile salts present in a subject to which the therapeutic agent is administered said Salmonella sp. is capable of inducing an immune response against itself or an antigen produced by itself.

More particularly, the present invention provides a therapeutic agent comprising S. dublin which carries a metabolic-drift mutation and has a substantially reduced capacity to grow and replicate due to bile salts present in, or introduced to, an environment within the host to which it migrates to following administration, wherein said S. dublin is capable of eliciting an immune response in a subject, which immune response is directed against an antigen produced by said S. dublin.

Microbiostatic agents can either be naturally present in the environment to which the present invention is administered or may be introduced, either simultaneously or sequentially, with the present invention at the time of administration. Sequential administration includes administration within nanoseconds, seconds, minutes, hours or days. Preferably, within seconds or minutes.

In one embodiment the present invention comprises a microorganism which only expresses naturally occurring antigens. However, the development of recombinant nucleic acid technology has facilitated the ability to modify the microorganisms of the present invention such that they also contain and/or express heterologous nucleic acid sequences.

Heterologous nucleic acid sequences may be introduced into the microorganism such that the nucleic acid remains extrachromosomal. The introduced nucleic acid sequences may also be in the form of artificial chromosomes, such as but not limited to bacterial artificial chromosomes (BACs). Alternatively, heterologous nucleic acid sequences may also incorporated directly into chromosomes naturally present within the microorganism. Methods for nucleic acid transfer to the microorganisms of the present invention are well known in the art (e.g. Sambrook and Russell, Molecular Cloning: A Laboratory Manual, 3^{rd} Edition, Cold Spring Harbor Laboratory Press, Cold Spring Harbor, N.Y., 2001; Ausubel (Ed), Current Protocols in Molecular Biology, 5^{th} Edition, John Wiley & Sons, Inc, NY, 2002) and include, but are not limited to, electroporation and calcium chloride-mediated transfection.

Heterologous nucleic acid sequences introduced to the microorganisms of the present invention facilitate “DNA vaccination” or allow the expression of heterologous proteins which may function, inter alia, as antigens or as factors which enhance the immunogenicity of a particular antigen.

The introduced heterologous nucleic acid sequences may co-exist and/or be co-expressed with other nucleic acid sequences already present in the microorganism of the present invention. This facilitates the co-expression of heterologous nucleic acid sequences, preferably in the form of antigens or factors which enhance the immunogenicity of a particular antigen, with the naturally occurring antigens on the microorganism of the present invention.

Accordingly, in another embodiment the present invention provides a therapeutic agent comprising a modified microorganism containing and/or expressing heterologous nucleic acid sequences which is substantially attenuated and incapable of growth and multiplication due to a microbiostatic agent present in, or introduced to, an environment within the host to which it migrates following administration.

Examples of proteins which may function as antigens include, but are not limited to, cell-surface proteins associated with microorganisms (e.g. bacteria, algae, fungi and protozoa) and viruses which cause upper respiratory tract infections, pleuropulmonary and bronchial infections, cardiovascular infections, urinary tract infections, sexually transmitted infections, nervous system infections, skin and soft tissue infections, gastrointestinal infections, bone and joint infections, eye infections and the like.

Examples of factors which may enhance the immunogenicity of a particular antigen include, but are not limited to, Brain-derived neurotrophic factor (BDNF), Cilliary-derived neurotrophic factor (CNTF), Epidermal growth factor (EGF), Erythropoitin (EPO), Fibroblast growth factor (FGF) 1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, Granulocyte colony stimulating factor (G-CSF), Granulocyte macrophage colony stimulating factor (GM-CSF), Interferon (IFN)α, IFNβ, IFNγ, Interleukin (IL) 1, IL2, IL3, IL4, IL5, IL6, IL7, IL8, IL9, IL10, IL11, IL12, IL13, IL14, IL15, Leukemia inhibitory factor (LIF), Monocyte chemotactic protein (MCP) 1, MCP2, MCP3, MCP4, MCP5, Macrophage colony stimulating factor (M-CSF), Macrophage inflammatory protein (MIP) 1, MIP2, Nerve growth factor (NGF), Neurotrophin (NT) 3, NT4, NT5, NT6, NT7, Oncostatin M (OSM), Platelet basic protein (PBP), Pre-B-cell growth stimulating factor (PBSF), Platelet derived growth factor (PDGF), Platelet factor (PF) 4, Regulated upon activation, normal T-cell expressed (RANTES), Sertoli cell factor (SCF), Transforming growth factor (TGF) α, TGFβ, Tumour necrosis factor (TNF) α, TNFβ, Thrombopoietin (TPO), Vascular endothelial growth factor (VEGF), Growth hormone (GH), insulin and the like.

The identification of therapeutic agents in the form of microorganisms capable of eliciting a humoral and/or cell-mediated immune response to cell surface antigens or antigens secreted or released from a cell enables these agents to be used in the prophylaxis and/or treatment of a range of diseases and conditions. Examples of diseases and conditions which may be prevented, ameliorated or treated by the therapeutic agents of the present invention include, but are not limited to, bacterial, viral, fungal and parasitic infections.

Reference herein to “treatment” may mean a reduction in the severity of an existing condition. The term “treatment” does not necessarily imply that a subject is treated until total recovery. Reference herein to the term “prophylaxis” means to prevent the onset of a particular disease or condition. Similarly, “prophylaxis” does not necessarily mean that the subject will not eventually contract a disease or condition.

Accordingly, the present invention further contemplates a method of vaccinating a subject against a microorganism or an antigen produced by a microorganism said method comprising selecting a microorganism, exposing the microorganism to naladixic acid and rifampicin or their chemical or functional equivalents for a time and under conditions sufficient to induce a metabolic drift mutation which renders the microorganism substantially unable to grow or replicate in the presence of a selected microbiostatic agent, and administering said mutated microorganism to the subject under conditions sufficient for the microorganism to migrate to an environment comprising the microbiostatic agent where it maintains itself for a time sufficient for an immune response to be induced to the microorganism or an antigen produced thereby.

The therapeutic agent of the present invention may be administered to a subject by any convenient means known to one skilled in the art. Routes of administration may include, but are not limited to, oral, intramuscular, intraperitoneal and parenteral routes.

The amount of therapeutic agent necessary to administer to a subject is an amount necessary at least partly to attain the desired response, or to delay the onset or inhibit progression or halt altogether, the onset or progression of a particular disease or condition being treated. The amount varies depending upon the species of the subject to be treated, the health and physical condition of the subject to be treated, the degree of protection desired, the assessment of the medical situation, and other relevant factors. It is expected that the amount will fall in a relatively broad range that can be determined through routine trials.

The therapeutic agent of the present invention is generally administered along with a pharmaceutically acceptable carrier or diluent. The particular pharmaceutically acceptable carrier or diluent used is not critical to the present invention. Examples of diluents include phosphate buffered saline, buffer for buffering against gastric acid in the stomach, such as citrate buffer (pH 7.0) containing sucrose, bicarbonate buffer (pH 7.0) alone (Levine et al., Journal of Clinical Investigation 79:888-902, 1987; Black et al., Journal of Infectious Diseases 155:1260-1265, 1987), or bicarbonate buffer (pH 7.0) containing ascorbic acid, lactose, and optionally aspartame (Levine et al., Lancet II:467-470, 1988). Examples of carriers include proteins such as those as found in skim milk, sugars such as sucrose, or polyvinylpyrrolidone. Typically these carriers would be used at a concentration of about 0.1-90% (w/v) but preferably at a range of 1-10% (w/v).

The present invention further contemplates the use of the microorganism herein described such as a Salmonella sp. and in particular S. dublin in the manufacture of a medicament to induce an immune response in a mammal to the microorganism.

The present invention is further described by the following non-limiting examples.

### Example 1

### General Experimental Procedures

The following experimental procedures are used in the subsequent examples which follow:

### Materials

### Bacteria

A S. dublin wild strain FD436 was obtained from the Department of Primary Industries' Animal Research Institute (ARI) and was used to generate metabolic-drift mutants. It was also the challenge strain used in mouse inoculation studies. This organism was the causative organism of an outbreak of salmonellosis in Beaudesert, Queensland, Australia in 1998.

### Chemicals

All chemicals were used in accordance with the manufacturer's recommendations. Information in square brackets indicates catalogue numbers of products.

### Commercial Chemicals

### Ajax Laboratory Chemicals (Ajax), New South Wales (NSW), Australia:

Glycerol, Sodium hydroxide pellets (analytical reagent (AR))

### BDH Chemicals (BDH), Victoria, Australia:

Acetic acid, Bromophenol blue, Ethanol, Glycine, Hydrochloric acid (HCl) sp.gr.1.18 (AR), Methanol 99.8 atom %, Sodium hydrogen carbonate (NaHCO₃) (AR)
Boehringer Ingelheim Pty. Ltd., Animal Health Division, NSW, Australia:
Nembutal (Pentobarbitone sodium)

### Gibco BRL, USA:

Ethylenediaminetetraacetic acid (EDTA), Sodium dodecyl sulfate (SDS), Tris

### LKB-Produkter, Sweden:

### Acrylamide

### Merck-Schuchardt, Germany

2-mercapto-ethanol

### Murex Biotech Ltd. (Murex), Dartford, England:

Salmonella polyvalent agglutinating serum (polyvalent 0 groups A-S) [ZC02]
Progen Industries Ltd., NSW, Australia, Agarose (DNA grade)
Sigma Chemical Co. (Sigma), St. Louis, Mo., USA:
Antimicrobials, Nalidixic acid [N-8878], Rifampicin approximately 95% [R-3501]

### Solutions

Phosphate buffered saline (PBS) ×1:

| | | |
|---|---|---|
| Sodium Chloride | 20 | g |
| Potassium Chloride | 0.5 | g |
| Na₂HPO₄ | 2.29 | g |
| KH₂PO₄ | 0.5 | g |
| Distilled water | 2500 | ml |

The mixture was sterilized by autoclaving at 121° C., 151b for 15 minutes (min).

### Equipment

Labsystems Co., Helsinki, Finland, Bioscreen analyser system

### Media

In the production of media, any necessary adjustments to pH were made with HCl or NaOH and media were autoclaved at 121° C., 151b for 15 min, unless otherwise stated.

### Commercial Media

Media were prepared following the manufacturer's recommendations and made up to volume with distilled water.

### Oxoid Ltd. (Oxoid), Hampshire, England

Brain-heart infusion (BHI) broth [CM225]
Cystine-Lactose-Electrolyte Deficient (CLED) medium [CM301]
Lysine decarboxylase (LD) broth (Taylor modification) [CM308]
MacConkey agar No. 3 (MCA) medium [CM1 15]
Mannitol selenite broth (MSB) base [L121]
Mueller-Hinton agar (MHA) medium [CM337]
Triple sugar iron (TSI) agar [CM277]
Tryptone soya broth (TSB) [CM129]
Xylose lysine desoxycholate (XLD) medium [CM469]

### Laboratory Made Media

### Cooked Meat (CM) Broth:

Beef hearts, from which fat and sinew had been removed, were minced. NaOH of 1/500 of the weight of the mince was dissolved in a volume of water equal to the weight of the mince. The mince was added to the NaOH solution and the mixture boiled for 20 min. After cooling, solid fat in the mixture was removed. The mixture was then filtered through gauze to remove any liquid. The dry mince was packed into 1 oz narrow bottles to about ¼ full. Bottles were filled with BHI broth and autoclaved.

### ONPG Broth:

### ONPG Solution:

| | |
|---|---|
| O-Nitrophenyl-β-D-galactopyranoside | 0.5 g |
| 0.1M potassium phosphate | 84.0 ml |

The solution was sterilised with a 0.45 U millipore filter and stored at 5° C. for up to 2 weeks.

### Basal Medium:

| | | |
|---|---|---|
| Tryptone [Oxoid, L42] | 1.0 | g |
| Sodium chloride | 0.5 | g |
| Distilled water | 100.0 | ml |

The pH of the medium was adjusted to 7.4 and the medium was autoclaved.

Twenty-five millilitres of the ONPG solution was aseptically added to 75 ml of the basal medium. The mixture was dispensed into ¼ oz bottles in 2 ml amounts and stored at 5° C. until use.

### Semi-Solid Medium:

The following ingredients were combined and heated to boiling point whilst stirring then cooled and the pH adjusted to 7.0.

| | | |
|---|---|---|
| ‘Lab-Lemco’ powder [Oxoid, L29] | 1.0 | g |
| Peptone bacteriological [Oxoid, L37] | 1.0 | g |
| Sodium chloride | 0.5 | g |
| Agar technical [Oxoid, L13] | 0.2 | g |
| Distilled water | 100 | ml |

### 10% Sheep Blood Agar (SBA):

### Blood Agar Base:

| | | |
|---|---|---|
| Blood agar base No. 2 [Oxoid, CM271] | 40 | g |
| Distilled water | 1000 | ml |

The blood agar base was autoclaved and kept at 56° C. until ready to pour then cooled to 50° C. Sterile sheep blood was mixed with the blood agar base at a ratio of 1:9. The mixture was poured onto plates and the plates dried in an incubator overnight. The plates were stored at 5° C. until use.

### Carbohydrate Fermentation Broth Media:

One millilitre of Andrade's indicator was added to 90 ml of 1% peptone water. One gram of each of the following carbohydrates was dissolved in 9 ml of distilled water and then added to the peptone water. These mixtures were then filtered through a millipore filter (0.2 μm). The mixture was dispensed into ¼ oz bottles in 2 ml amounts and stored at room temperature in a dark room until use.

0.04% (w/v) Nalidixic Acid and/or 0.02% (w/v) Rifampicin SBA:
2% (w/v) Nalidixic Acid Solution:

| | | |
|---|---|---|
| Nalidixic acid | 200 | mg |
| 1N NaOH | 2 | ml |
| Sterilised distilled water | 8 | ml |

1% (w/v) Rifampicin Solution:

| | | |
|---|---|---|
| Rifampicin | 100 | mg |
| Methanol (99.8 atom %) | 10 | ml |

The blood agar base was autoclaved and kept at 56° C. until ready to pour then cooled to 50° C. Sterile sheep blood was mixed with the blood agar base at a ratio of 1:9. One millilitre of either 2% nalidixic acid or 1% rifampicin solution, or both were then added to a mixture of the blood agar base and the sheep blood to make a total volume of 50 ml. The mixture was poured onto the plates and the plates dried in an incubator overnight. The plates were stored at 5° C. until use.

### Experimental Animals

### Mice:

Unless otherwise stated, conventional female mice (20-25 g) were purchased from the UQ Central Animal Breeding House. To minimise the influence of individual variation on experimental results, mice with the same genetic line were used. The mice were housed in standard mice cages (the number of mice in each cage was equal to or less than five) and were given commercial mouse pellets and clean water. Health of the mice was monitored once a day (9:00 AM) for 3 days before commencing experiments. Twenty pieces of faeces in each cage were randomly taken everyday over this period and tested using the Salmonella isolation and identification methods to confirm the mice were free from Salmonella. The bedding was renewed every day and the cages were sterilised with 70% ethyl alcohol and then flamed at each renewal. Mice showing any clinical signs were isolated from other healthy mice immediately after the finding and replaced by healthy mice.

### Methods

### Bacteria Storage:

One colony from a pure culture was inoculated onto a 10% (v/v) SBA plate then incubated at 37° C. for 24 hours (h). Bacteria growing on the plate were mixed with BHI broth to obtain a suspension of approximately 10⁹ bacteria per ml. Aliquots of 0.5 ml each were then distributed into small vials and stored at −70° C. until required, at which time aliquots were cultured once on SBA at 37° C. for 24 h and then used for the experiment, unless otherwise stated.

### Bacterial Enumeration:

The number of viable bacterial cells in a specimen was estimated as cfu using the Miles-Misra surface plate-count method (Miles et al., Journal of Hygiene Cambridge 38:732-749, 1938), unless otherwise stated. Ten-fold dilution of specimens containing Salmonella were made in PBS. One hundred microliters of each dilution were inoculated onto SBA plates. For estimating the number of nalidixic acid and/or rifampicin resistant mutants, 0.04% (w/v) nalidixic acid and/or 0.02% (w/v) rifampicin SBA plates were also used. These plates were incubated at 37° C. for 24 to 48 h before counting the number of colonies on the plates.

### Salmonella Isolation:

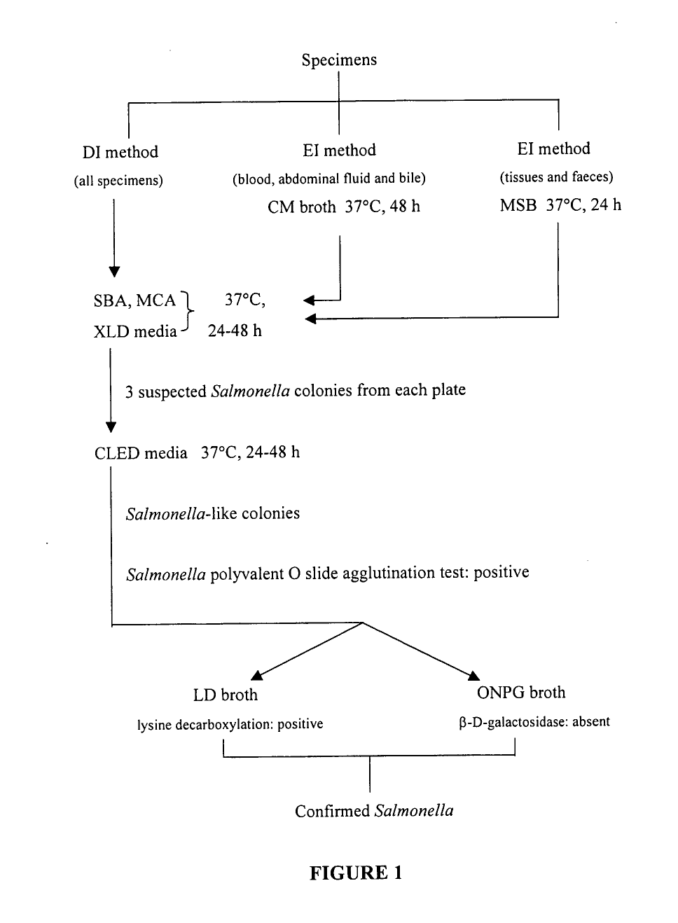

Unless otherwise stated, three methods were employed to isolate Salmonella including its metabolic-drift mutants. When the organisms were not isolated through the direct isolation (DI) method, the enrichment isolation (EI) methods were employed (FIG. 1).

DI method for all specimens: Specimens were directly plated onto SBA, MCA and XLD plates. These media were then incubated at 37° C. for 24 to 48 h.

EI method for blood, abdominal fluid and bile specimens: A specimen was added to 20 ml of CM broth and incubated at 37° C. After 48 h, 50 μl of the broth were plated onto SBA, MCA and XLD plates. These plates were then incubated at 37° C. for 24 to 48 h.

EI method for tissue and faecal specimens: Up to 1 ml of homogenate of each sample was added to 10 ml of MSB containing 0.0001 g L-cystine (BDH) and incubated at 37° C. for 24 h. Fifty microlitres of MSB were then plated onto SBA, MCA and XLD plates. These plates were incubated at 37° C. for 24 to 48 h.

### Salmonella Identification:

After isolation, three colonies suspected of being Salmonella from each XLD plate were transferred to CLED media. After incubation for 48 h, colonies suspected of being Salmonella were then tested serologically in the polyvalent 0 antigen slide agglutination test. Colonies which produced a positive result to this test were further tested by culturing in LD and ONPG broths. Colonies that showed lysine decarboxylation and an absence of β-D-galactosidase were identified as Salmonella (FIG. 1).

Identification of Nalidixic Acid and/or Rifampicin Resistant Strains:

Bacteria identified as Salmonella were further investigated to determine their resistance to either nalidixic acid or rifampicin, or both. The bacteria were inoculated onto: 1) SBA plates containing 0.04% nalidixic acid; 2) SBA plates containing 0.02% rifampicin, and; 3) SBA plates containing 0.04% nalidixic acid and 0.02% rifampicin. These plates were incubated at 37° C. for 24 to 48 h. Colonies growing on 1) but not growing on 2) and 3) were identified as resistant to nalidixic acid, those growing on 2) but not on 1) and 3) as resistant to rifampicin and those growing on 1), 2) and 3) as resistant to both nalidixic acid and rifampicin.

### Polyvalent O Antigen Slide Agglutination Test for Salmonella:

Bacterial colonies were mixed with a drop of sterile PBS to make two heavy suspensions per slide. One of the suspensions was left for one minute to confirm the absence of auto-agglutination, then a drop of PBS was added to the suspension as a control. A drop of Salmonella polyvalent O agglutinating serum was mixed with the other bacterial suspension. The slide was then rocked in a rolling motion for one minute. Suspensions in which agglutination occurred within one minute were considered positive.

### MIC and MBC of Antimicrobial Agents:

MIC was defined as the lowest concentration of antimicrobial agents which inhibits visible bacterial growth, and MBC is the lowest concentration of the agents which kills a defined proportion (usually 99.9%) of the bacterial population after incubation for a set time (Collins et al., Microbiological Methods pp 86, 114-115, 178-205, Butterworth-Heineman, Oxford, England, 1995). The broth dilution method was used for determining MIC. The method for determining MBC was an extension of the broth dilution method.

### Determination of Bacterial Growth Rate

### Bioscreen Analyser System:

All microorganisms increase the turbidity (optical density (OD)) of the liquid growth medium in which they are being cultured, and for any given type of microorganism, the relationship between the turbidity and the microorganism concentration in a liquid medium is approximately linear over a certain range of values (Metssalu, A new user's manual, pp 1-27, Labsystems Corporation, Helsinki, Finland, 1995). Bioscreen is a fully automated, turbidmetric instrument to observe in vitro growth of microorganisms kinetically or at an end-point. To determine kinetic growth of microorganisms, the turbidity of samples is periodically monitored at the selected temperatures (room temperature, 37 and 40° C.) for the desired period by a vertical light photometry system. In this system, a vertical light beam passes through the bottom of the cuvette, the sample, and the cuvette cover to the photometric reader. A turbidity curve (growth curve) is computer generated based on the results of turbidimetry.

### Determination of Growth Rate:

Unless otherwise stated, the following procedure was used in the project. Bacteria were suspended in a liquid medium to a concentration of approximately 10⁸ cfu/ml. In order to prevent an extended lag phase, suspensions were pre-incubated aerobically at 37° C. for 3 h (for wild strain FD436) and 5 h (for metabolic-drift mutants). The same type of liquid medium (heated to 37° C.) was then added to obtain a concentration of approximately 10⁵ cfu/ml. Two hundred microlitres of well-mixed suspension were aseptically transferred into a sterile cuvette. The cuvette was then immediately placed in Bioscreen and the turbidity of the suspension measured at the required temperature over time. A light wavelength filter for a wide band area (420-580 nm) was used because results obtained using this filter are not affected by changes in the colour of the growth media but indicate only the development of turbidity (Metssalu, 1995, Supra). The growth rate was taken to be the median value of three simultaneous measurements for each strain.

### Replica Plate Method:

A disk-shaped plate with a stand which fits inside a petri dish was used to aseptically replicate large numbers of colonies growing on one medium to fresh solid media. Before replication, the surface of the disk was tightly covered with a piece of sterile cloth. The covered surface was then applied evenly and precisely to the agar surface containing between 30 and 300 colonies. The disk was then applied evenly to the new media, accurately reproducing colony distribution on the original plate (Carlton and Brown, Gene Mutation in Manual of Methods for General Bacteriology, pp 232-234, Eds Gerhardt et al., American Society for Microbiology, Washington D.C., USA, 1981).

### Challenge Bacteria and Vaccine Preparation:

Fresh cultures were suspended in PBS to give an approximate concentration of 10⁷ cfu/ml. One millilitre of suspension was flooded onto SBA plates, reduced by anaerobic incubation at 37° C. overnight, and the plates then anaerobically incubated at 37° C. for 4 h (for wild strain FD436) and 6 h (for metabolic-drift mutants). Bacteria growing on each plate were harvested in PBS by gentle washing using a pipette. Bacterial preparations for challenge infection and vaccination were made from these suspensions by adding warm (37° C.) PBS to give the desired bacterial concentrations.

### Fifty Percent Infectious Dose (ID₅₀):

The ID₅₀ is a measurement of microorganism virulence that determines the dose required to infect 50% of the animals in the target group. Groups of forty mice were used to estimate the ID₅₀ and to provide a standard deviation from the mean using the Reed-Muench method (Reed and Muench, The American Journal of Hygiene 27:493-497, 1937).

### Euthanasia of Mice:

Five parameters, activity, appetite, dehydration, respiratory rate and faecal consistency, were monitored to assess the health of mice. Mice showing clinical signs in three or more of the five parameters were classed as suffering from serious illness and immediately euthanased using intraperitoneal injection with Nembutal at a dose of 100 μl/mouse.

### Statistical Methods:

GraphPad Instat version 3.05 (GraphPad Software, San Diego, USA) was used to analyse data. Results were considered statistically significant when probability was less than 5% (p<0.05). Detailed statistical analysis is described in each chapter.

### Example 2

### Isolation of Double Metabolic-Drift Mutants

Experimental procedures for Example 2:

### Bacteria:

Fresh cultures of FD436 were prepared as previously described.

### MIC and MBC Determination

### Media:

Two percent (w/v) nalidixic acid solution, or 1% (w/v) rifampicin solution, was added to TSB to make 2 ml of TSB media containing graded concentrations of nalidixic acid or rifampicin (0.04, 0.02, 0.01, 0.005, 0.0025, 0.00125, 0.000625 and 0.0003125% (w/v)). TSB media containing no antimicrobials were also prepared as controls. TSB medium was used instead of the conventionally recommended media for MIC and MBC determinations as metabolic-drift mutants may be more fastidious than their parent strains and thus may require highly nutritious media, such as TSB.

### Methods:

Turbidity (OD₄₂₀₋₅₈₀) measured by Bioscreen and visual observation after 24 h incubation at 37° C. were the criteria used for assessing the growth of bacteria. FD436 was mixed with the above TSB media containing the graded concentrations of antimicrobials to make approximately 10⁶ cfu/ml bacterial suspensions (the actual concentration was 9.6×10⁵ cfu/ml). Inoculated and uninoculated TSB containing no antimicrobials were included as positive and negative controls, respectively. For determining the MIC, 200 μl of each medium was aseptically transferred to a sterile cuvette and bacterial growth was measured at 37° C. for 24 h using Bioscreen. In addition, the remainder of the medium was incubated aerobically at 37° C. for 24 h for visual observation of turbidity. The MICs of the antimicrobials were determined based on the definition described previously. For determining the MBC, each medium containing antimicrobial concentrations above the MIC was diluted with TSB to the MIC, and 1 ml of each was pipetted directly onto an SBA plate and distributed with a sterile glass spreader. These plates were incubated at 37° C. for 48 h. Colonies growing on the SBA were identified using the Salmonella identification procedure. The MBC of each antimicrobial was determined based on the definition described previously.

### Isolation of Single Antimicrobial-Resistant Strains:

Colonies from a fresh culture of FD436 on SBA plates were mixed with PBS to make a bacterial suspension of approximately 10¹⁰ cfu/ml (the actual concentration was determined to be 2.1×10¹⁰ cfu/ml). One millilitre of the suspension was spread onto SBA plates containing 0.04% (w/v) nalidixic acid or 0.02% (w/v) rifampicin (three plates each), and a SBA plate containing no antimicrobials as a control. These plates were incubated aerobically at 37° C. for 72 h. Bacteria isolated on the plates were identified using the Salmonella identification procedure.

### Selection of Stable, Single Antimicrobial-Resistant Strains

Seventeen strains (N1-N14 and R1-R3) were tested for stability of resistance by subculturing a total of 10 times on SBA plates containing 0.04% (w/v) nalidixic acid and 0.02% (w/v) rifampicin, respectively. Each strain was also inoculated onto SBA plates containing no antimicrobials as a control. Plates were incubated aerobically at 37° C. for 48 h before the next subculture.

Selection of Single Antimicrobial-Resistant Strains with a Reduced Growth Rate:

The turbidity of strains N1-N14 and R1-R3 growing in TSB media at 37° C. was measured every 10 min for 18 h using Bioscreen. The determination of turbidity followed the procedure described previously. The turbidity of strain FD436 growing in TSB media was also measured as a control in the same manner. The growth rate during the logarithmic growth phase of each strain was compared.

### Isolation of Double Antimicrobial-Resistant Strains:

Colonies from fresh cultures of strains N1, N6 and R3 on SBA plates were mixed with PBS to make bacterial suspensions of approximately 10¹⁰ cfu/ml (actual concentrations were 1.6×10¹⁰, 1.1×10¹⁰, 1.6×10¹⁰ and 0.9×10¹⁰ cfu/ml, respectively). One millilitre of each suspension containing the nalidixic acid-resistant strains was spread onto 0.04% (w/v) nalidixic acid and 0.01% (w/v) rifampicin SBA plates and 0.04% (w/v) nalidixic acid and 0.02% (w/v) rifampicin SBA plates (three plates each). One millilitre of the suspension containing the rifampicin-resistant strain was spread onto 0.02% (w/v) nalidixic acid and 0.02% (w/v) rifampicin SBA plates and 0.04% (w/v) nalidixic acid and 0.02% (w/v) rifampicin SBA plates (three plates each). The same amount of the suspensions was inoculated onto SBA plates containing no antimicrobials as controls. All plates were incubated aerobically at 37° C. for 72 h. Bacteria growing on each plate were identified using the Salmonella identification procedure.

### Selection of Stable, Double Antimicrobial-Resistant Strains:

Twenty-seven potential N-R double antimicrobial-resistant mutant colonies were randomly selected from 94 colonies growing on 0.04% (w/v) nalidixic acid and 0.02% (w/v) rifampicin SBA plates on which N1 or N6 had been inoculated. As only 2 potential R-N double antimicrobial-resistant mutant colonies were isolated both were selected for further studies. The selected colonies and ID numbers of the colonies (strains) are indicated in Table 6. Each candidate double antimicrobial-resistant strain (1-29) was subcultured 10 times on SBA containing 0.04% (w/v) nalidixic acid and 0.02% (w/v) rifampicin, and on SBA containing no antimicrobials (control). Each subculture was incubated aerobically at 37° C. for 48 h. The strains were then subcultured a further 10 times on SBA containing no antimicrobials (each subculture was incubated at 37° C. for 48 h). Following this, an isolated colony of each strain was mixed with 2 ml of PBS and the serial ten-fold dilution method was employed to make bacterial suspensions containing a final concentration of approximately 10² cfu/ml. One millilitre of each suspension was spread onto individual SBA plates and incubated aerobically at 37° C. for 48 h. After incubation, colonies growing on each SBA plate were transferred onto 0.04% (w/v) nalidixic acid and 0.02% (w/v) rifampicin SBA plates and new SBA plates using the replica plate method. The replicated plates were then incubated aerobically at 37° C. for 48 h. Approximately 500 colonies of each strain were transferred using the replica plate method.

Selection of Double Antimicrobial-Resistant Strains with a Reduced Growth Rate:

The growth rates of double antimicrobial-resistant strains 1-29 were determined based on the results of turbidimetry measurements by using the same procedures as outlined previously.

### Results for Example 2

### MIC and MBC Determination for FD436

### MIC and MBC of Nalidixic Acid:

Through turbidity measurements obtained with Bioscreen and direct observation, the MIC of nalidixic acid for FD436 was determined to be 0.00125% (w/v) (Table 3). The MBC was also determined to be 0.00125% (w/v) as profuse growth (greater than 1.0×10³ colonies) of Salmonella was observed on SBA plates streaked with TSB media originally containing equal to or less than 0.000625% (w/v) of nalidixic acid. Only small numbers of colonies or no colonies at all were found on SBA plates streaked with TSB media originally containing equal to or greater than 0.00125% (w/v) of the antimicrobial (Table 4).

### MIC and MBC of Rifampicin:

Through testing with Bioscreen and direct observation, the MIC of rifampicin for FD436 was determined to be 0.0025% (w/v) (Table 3). The MBC was also determined to be 0.0025% (w/v) (Table 4).

TABLE 3
TURBIDITY OF TSB MEDIA CONTAINING NALIDIXIC ACID OR
RIFAMPICIN AFTER 24-HOUR INCUBATION MEASURED USING
BIOSCREEN

| Concentration of antimicrobial in TSB | | |
|---|---|---|
| (% (w/v)) | Nalidixic acid | Rifampicin |
| 0.04 | − | − |
| 0.02 | − | − |
| 0.01 | − | − |
| 0.005 | − | − |
| 0.0025 | − | − |
| 0.00125 | − | + |
| 0.000625 | + | + |
| 0.0003125 | + | + |
| Positive control | + | + |
| Negative control | − | − |

+ = increase,
− = no change

TABLE 4
GROWTH OF Salmonella ON SBA PLATES AFTER 48-HOUR
INCUBATION IN TSB MEDIA CONTAINING NALIDIXIC ACID OR
RIFAMPICIN.

| Concentration of antimicrobial in TSB | | |
|---|---|---|
| (% (w/v)) | Nalidixic acid | Rifampicin |
| 0.04 | 1 | N.G |
| 0.02 | 8 | 1 |
| 0.01 | N.G | 1 |
| 0.005 | N.G | 5 |
| 0.0025 | 1 | 6 |
| 0.00125 | N.G | P.G |
| 0.000625 | P.G | P.G |
| 0.0003125 | P.G | P.G |
| Positive control | P.G | P.G |
| Negative control | N.G | N.G |

N.G = no growth,
P.G = profuse growth (greater than 1.0 × 10³ colonies)

### Isolation and Stability of Single Antimicrobial-Resistant Strains:

In total, 14 colonies were observed on the SBA plates containing 0.04% (w/v) nalidixic acid after 72 h of incubation. After 72 h of incubation, 3 colonies were found on the SBA plates containing 0.02% (w/v) rifampicin. Similar to the growth observed on the 0.04% (w/v) nalidixic acid SBA plates, the colonies on the 0.02% (w/v) rifampicin SBA plates took more time to appear than the colonies on the control plate. Each isolated colony was identified as a strain of Salmonella. Strains from SBA plates containing nalidixic acid were designated N1-N14 and strains from SBA plates containing rifampicin were designated R1-R3.

All strains grew well on SBA plates containing the antimicrobials throughout the ten subcultures.

Selection of Single Antimicrobial-Resistant Strains with a Reduced Growth Rate:

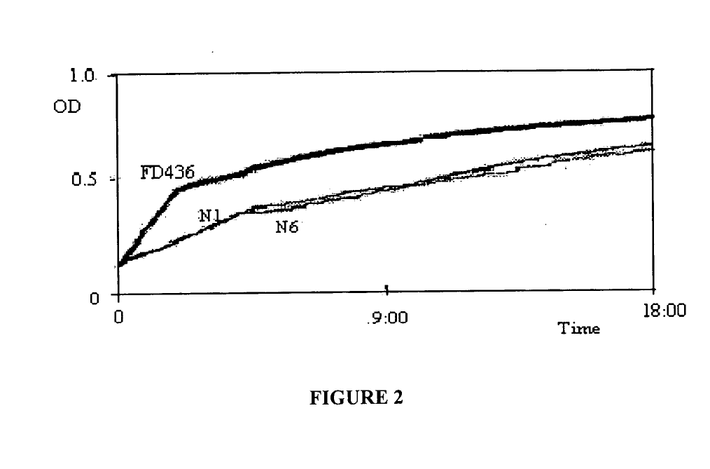

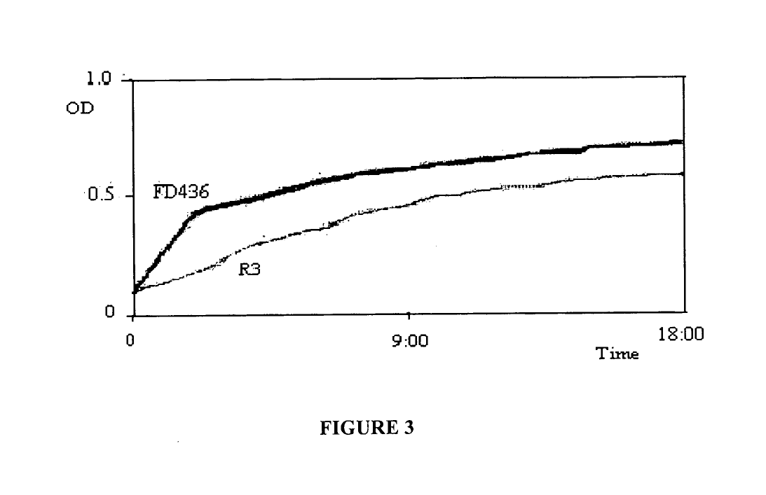

Nalidixic acid resistant strains N1 and N6 had slower growth rates when compared with wild strain FD436 (FIG. 2). Rifampicin resistant strain R3 also had a reduced growth rate when compared with FD436 (FIG. 3). Based on these results, N1, N6 and R3 were selected for producing double antimicrobial-resistant strains.

### Isolation of Double Antimicrobial-Resistant Strains:

For N1 and N6, the total number of potential double antimicrobial-resistant mutant colonies ranged between 38 and greater than 100 for each antimicrobial concentration after 72 h of incubation. For R3, only one colony grew on each plate for each antimicrobial concentration after 72 h of incubation (Table 5). Profuse growth was seen on each control plate. Bacteria growing on the plates were identified as Salmonella.

TABLE 5
NUMBER OF POTENTIAL DOUBLE ANTIMICROBIAL-
RESISTANT MUTANT COLONIES (N-R AND R-N)
GROWING ON SBA PLATES CONTAINING DIFFERENT
CONCENTRATIONS OF NALIDIXIC ACID AND
RIFAMPICIN AFTER 72-HOUR INCUBATION.

| | Concentration (w/v) of antimicrobials | Number of |
|---|---|---|
| Inoculated strain | in media | colonies* |
| N1 | 0.04% nalidixic acid & 0.01% | >100 |
| | rifampicin | |
| N1 | 0.04% nalidixic acid & 0.02% | 56 |
| | rifampicin | |
| N1 | No antimicrobials (control) | P.G |
| N6 | 0.04% nalidixic acid & 0.01% | 59 |
| | rifampicin | |
| N6 | 0.04% nalidixic acid & 0.02% | 38 |
| | rifampicin | |
| N6 | No antimicrobials (control) | P.G |
| R3 | 0.02% nalidixic acid & 0.02% | 1 |
| | rifampicin | |
| R3 | 0.04% nalidixic acid & 0.02% | 1 |
| | rifampicin | |
| R3 | No antimicrobials (control) | P.G |

P.G = profuse growth,
N.G = no growth,
*= Numbers indicate the total number of colonies on 3 plates.

### Selection of Stable, Double Antimicrobial-Resistant Strains:

The twenty-nine potential double antimicrobial-resistant strains (Table 6) grew well and maintained their double antimicrobial resistance throughout and following the ten subcultures on SBA plates containing 0.04% (w/v) nalidixic acid and 0.02% (w/v) rifampicin. Therefore, the selected strains have obtained highly stable resistance to both nalidixic acid and rifampicin.

TABLE 6
CANDIDATE DOUBLE ANTIMICROBIAL-RESISTANT STRAINS
SELECTED IN ORDER TO DETERMINE THE STABILITY OF
RESISTANCE.

| Parent | | | | |
|---|---|---|---|---|
| single | | | | |
| anti- | Concentration | Order of | | |
| microbial- | (w/v) of | obtained | Number of | |
| resistant | antimicrobials in | antimicrobial- | selected | ID |
| strain | media | resistance | colonies | number |
| N1 | 0.04% nalidixic acid & | N-R | 12 | 1-12 |
| | 0.02% rifampicin | | | |
| N6 | 0.04% nalidixic acid & | N-R | 15 | 13-27 |
| | 0.02% rifampicin | | | |
| R3 | 0.02% nalidixic acid & | R-N | 1 | 28 |
| | 0.02% rifampicin | | | |
| R3 | 0.04% nalidixic acid & | R-N | 1 | 29 |
| | 0.02% rifampicin | | | |

Selection of Double Antimicrobial-Resistant Strains with a Reduced Growth Rate:

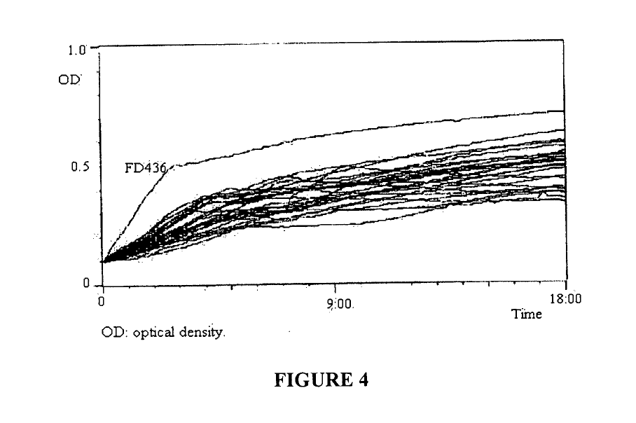

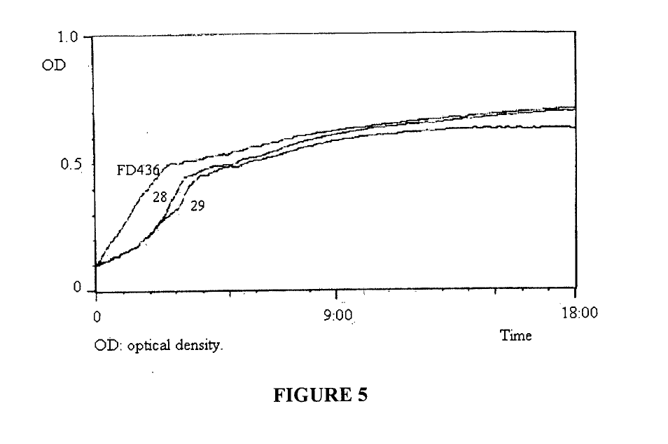

Compared with wild strain FD436, the growth rates of both the N-R and the R-N double antimicrobial-resistant strains were reduced to different levels (FIGS. 4 and 5).

### Example 3

### Characterization of Double Antimicrobial-Resistant Strains with Reduced Growth Rate and Selection of Vaccine Candidates

### Experimental Procedures for Example 3

### Bacteria:

FD436 and the 8 selected double antimicrobial-resistant strains N-RM4, 8, 9, 15, 20, 25, and 27 and R-NM29 were prepared following the procedure described previously.

### Genotyping Characterization:

Genotypic characterisation of FD436 and the double antimicrobial-resistant strains prepared as above was performed. To investigate the stability of the identified mutations, characterisation of the strains was repeated following subculturing 15 times on SBA (each subculture was incubated at 37° C. for 48 h).

PCR amplification of S. dublin gyrA Genes:

A portion of the gyrA gene (expected approximate size 347 base pairs (bp) (Griggs et al., Antimicrobial Agents and Chemotherapy 40:1009-1013, 1996) encompassing the QRDR region was amplified by PCR for FD436 and the 8 mutant strains using 20-mer oligonucleotide primers, SALGYRA-F and SALGYRA-R (synthesized by Genset Pacific Pty., Ltd., Lismore, Australia). The primer sequences were as follows—SALGYRA-F=5′-TGTCCGAGATGGCCTGAAGC-3′ (SEQ ID NO:1) and SALGYRA-R=5′-TACCGTCATAGTTATCCACG-3′ (SEQ ID NO:2). The PCR amplification reactions (total 50 μl) contained 6.4 picomole (pmol) of each primer, 200 μM of each dinucleotide triphosphate (dNTP), 1× Expand High Fidelity buffer with 2 mM magnesium chloride (Expand High Fidelity PCR System, Roche Diagnostics Australia Pty., Ltd., Australia), I unit of Expand High Fidelity PCR enzyme mix (Expand High Fidelity PCR System) and sterile distilled water. For ease and rapidity, all PCR assays were performed directly using single colonies grown on SBA plates as the DNA target. A small amount of a colony was taken by touching it lightly with a small pipette tip, and this sample was then resuspended in the PCR amplification reaction. All amplification reactions were performed in a Gene Amp PCR System 2400 thermocycler (PerkinElmer, USA). After an initial denaturation at 94° C. for 3 min, the cycling protocol for 30 cycles was as follows: denaturation at 94° C. for 30 seconds (sec), annealing at 55° C. for 30 sec and extension at 68° C. for 3 min. A final extension was conducted at 72° C. for 3 min.

PCR Amplification of S. dublin rpoB Genes:

A portion of the rpoB gene (expected approximate size 708 bp (McClelland et al., Nature 413:852-856, 2001)) was amplified by PCR for FD436 and the mutant strains. This portion encompassed three distinct clusters (I, II and III) that Jin and Gross (Journal of Molecular Biology 202:45-58, 1988) suggest are related to rifampicin resistance. Materials and methods for this amplification were identical to those described previously for PCR amplification of the gyrA gene with the exception of the type of primers used. In this experiment, 20-mer oligonucleotide primers, SALRPOF3 and RPOBREV (Genset Pacific Pty., Ltd. and Gibco BRL, respectively) were used. The primer sequences were as follows—SALRPOF3=5′-TCGGCAACCGTCGTATCCGT-3′ (SEQ ID NO:3) and RPOBREV=5′-TCGCACCCATCAACGCACGG-3′ (SEQ ID NO:4).

### Agarose Gel Electrophoresis:

Amplified DNA fragments were separated by agarose gel electrophoresis in 2% and 1.5% agarose gels containing 1% ethidium bromide (Bio-Rad Laboratories Ltd., Sydney, Australia). The gels were prepared by dissolving the desired amounts of DNA Grade Agarose in 1×TAE buffer (40 mM Tris base) and 1 mM EDTA titrated to pH 7.9 with acetic acid) by heating in a microwave oven. The dissolved agarose was then cooled to approximately 50° C. and poured onto a flatbed horizontal gel apparatus (Bio-Rad Laboratories Ltd.). Before use, the agarose was kept at room temperature for 30 min to allow polymerisation.

Following the above amplifications, 5 μl of each PCR product was mixed with 1 μl of tracking dye solution (50% glycerol, 0.1% bromophenol blue and 0.1% xylene cyanol FF (Australian Chemical Reagents, Australia)), loaded into a well and electrophoresed in the agarose gels (2% for gyrA products and 1.5% for rpoB products) at 60 mA for 1 h. A 1 kbp DNA ladder (MBI Fermentas, USA) was used as a standard. Amplified DNA fragments were visualised by UV transillumination.

### DNA Sequencing and Analysis:

Prior to DNA sequencing, PCR products were purified using the QIAquick PCR Purification Kit (Qiagen, Germany) following the procedure described in the manufacturer's manual. The oligonucleotide primers, SALGYRA-F and SALGYRA-R, and SALRPOF3 and RPOBREV, were used as primers for direct sequencing of the amplified gyrA and rpoB gene fragments, respectively. Each sequencing reaction mixture (total 12 μl) contained 4 μl of BigDye Ready Reaction DyeDeoxy Terminator Cycle Sequencing kit (Applied Biosystems, USA), 3.2 μmol of primer, 1 μl of purified PCR product (DNA product) and sterile distilled water. Sequencing reactions were performed using the following cycling conditions: initial denaturation at 94° C. for 5 min; 30 cycles of denaturation at 96° C. for 10 sec, annealing at 50° C. for 5 sec and extension at 60° C. for 4 min. Products of the sequencing reactions were purified using the following procedure. Each sequencing reaction mixture (12 μl) was mixed with 50 μl of ice-cold 100% ethanol and 2 μl 3M sodium acetate (Sigma), and kept at −20° C. for 30 min. The mixtures were then centrifuging performed (13,000 rpm at 4° C.) for 30 min and the supernatant was gently discarded to obtain a pellet containing the sequencing reaction product. Five hundred microliters of 70% ethanol was added and centrifuged again (13,000 rpm, 4° C., 30 min). Supernatant was carefully discarded and the pellet was air-dried. The reaction product was analysed using a 373A DNA Sequencing System at the Australian Genome Research Facility (AGRF), Brisbane, Qld.

### Phenotypic Characterization

### Colony:

Colonies on SBA plates seeded with each of the 9 strains and incubated aerobically for 24 h at 37° C. were examined under 10× magnification.

### Motility:

The conventional hanging drop method (Frost and Spradbrow, Veterinary Microbiology I, II, II Practical Manual and Laboratory Notes, p 8, The University of Queensland, Brisbane, Australia) and the Craigie tube method (Collins et al., Supra) were used to examine the motility of FD436 and the selected mutants.

For the Craigie tube method, 12 ml of semi-solid medium was distributed into one oz screw-capped bottles containing an open-ended glass tube (the Craigie tube) to make a Craigie tube set. These sets were then autoclaved at 121° C. for 15 min. One individual colony of each strain was inoculated approximately 10 mm below the surface of the semi-solid medium within the Craigie tube using a sterile straight wire. The media were then incubated at 37° C. for 36 h. Samples of each strain were taken every 3 h from the surface of the media outside the Craigie tube and cultured on XLD plates. To avoid false positive results produced by bacterial contamination, organisms growing on the XLD media were identified as being Salmonella using the procedure described previously. Each result was the mean of two separate determinations per strain.

### Capsulation:

Duguid's method (Doetsch, Determinative Methods of Light Microscopy In Manual of Methods for General Bacteriology, p 29, Eds Gerhardt, et al., American Society for Microbiology, Washington D.C., USA, 1981) was used to determine capsulation. Briefly, a large loopful of India ink was mixed with cells from each strain on a clean slide and then pressed down with a glass cover slip. Excess mixture was absorbed by blotting paper. Capsulation was examined with high-dry (×400) and oil immersion lens systems.

### Lipopolysaccharides (LPS)

Lipopolysaccharides extracts were resolved by SDS-PAGE and silver stained to detect and evaluate heterogeneity of LPS structure between the strains.

### Extraction of LPS:

Proteinase K digestion (Hitchcock and Brown, Journal of Bacteriology 154:269-277, 1983) followed by phenol-water extraction (Marolda et al., Journal of Bacteriology 172:3590-3599, 1990) was employed to extract LPS. Colonies from fresh cultures of each strain were suspended in PBS (pH 7.2) and the concentration adjusted to approximately 10⁹ cfu/ml. To sediment bacteria, 1.5 ml of each suspension was centrifuged at 10,000×g for 3 min. Supernatant fluid was carefully discarded and the pellet was resuspended in 50 μl of solubilisation buffer (Table 7). This suspension was heated at 100° C. for 10 min. Twenty-five micrograms of proteinase K (Roche Diagnostics GmbH, Germany) was then added to the suspension and incubated at 60° C. for 1 h. The suspension was extracted with double the volume of TE Saturated Phenol/Chloroform (Progen Industries Ltd.) by stirring vigorously at 65° C. for 15 min. The suspension was then chilled on ice and centrifuged at 8,500×g for 10 min. The aqueous phase was transferred to a new microcentrifuge tube, and LPS was precipitated twice with 10 volumes of 95% ethanol at −20° C. for 6 h. The precipitate was suspended in 50 μl of DDW and stored at −20° C. until use.

### SDS-PAGE of LPS Extracts:

SDS-PAGE was performed for the selected mutants and the wild strain using the bi-layer stacking gel method (Inzana and Apicella, Electrophoresis 20:462-465, 1999) with some modifications. Gels were cast in 18 cm×16 cm glass plates separated with 0.75 mm spacers. The separating gel (Table 6) was poured to 4 cm from the top of the plates. After the separating gel polymerised, stacking gel A (Table 6) was poured onto the separating gel to a level at which the well comb just touched stacking gel A. After polymerisation, the well comb (0.75 mm) was placed in the gel sandwich and then overlaid with stacking gel B (Table 6) until all the teeth were covered by the gel. The well comb was carefully removed after polymerisation of the gels and the wells were filled with reservoir buffer (0.025M Tris base/0.19M glycine, pH 8.3) containing 0.1% sodium dodecyl sulfate (SDS).

Prior to running the gel, extracted LPS of each sample in 50 μl of DDW was mixed with the same amount of solubilisation buffer and placed in boiling water for 5 min. Fifty microliters of each mixture was loaded into the well. Electrophoresis was performed at 4° C. using a circulating cooling bath in a Protean II vertical electrophoresis unit (Bio-Rad Laboratories Ltd.). The samples were electrophoresed at a constant current of 10 mA/gel through both stacking gels, then 15 mA/gel through the separating gel. Two gels were prepared for the samples and electrophoresed at the same time under the same conditions. One of the gels was stained with ammoniacal silver for detecting LPS. To confirm complete digestion of protein in the samples by Proteinase K, the other gel was stained with Coomassie Brilliant Blue stain (10% (v/v) methanol, 10% (v/v) acetic acid and 0.1% (w/v) Coomassie Blue R-250 (Sigma).

### Silver Staining:

After electrophoresis, the gel was fixed and stained with ammoniacal silver following the method described by Tsai and Frasch (Analystical biochemistry 119:115-110, 1982) with some modification. The gel was fixed overnight with 200 ml of 40% (v/v) ethanol and 10% (v/v) acetic acid in DDW. The gel was then oxidised for 5 min in 200 ml of the above fixative containing 0.7% (w/v) periodic acid (Sigma), and washed with DDW three times for 15 min each. The gel was stained for 10 min with a freshly prepared stain consisting of 0.25 ml of 30% sodium hydroxide (Ajax), 1.4 ml of 14.8M ammonium hydroxide (Sigma), 4 ml of 20% silver nitrate (Ajax) and 97 ml of DDW. After staining, the gel was washed with DDW three times for 10 min each and then developed with 0.005% (w/v) citric acid (Ajax) and 0.019% (v/v) formaldehyde (Asia Pacific Specialty Chemicals Ltd., Australia) in DDW. When bands were visible, the development was stopped by removing the gel and soaking in the fixative.

TABLE 7
COMPOSITION OF SOLUBILISATION BUFFER AND
SEPARATING AND STACKING GELS.

| Solution | Component | Volume (ml) |
|---|---|---|
| Solubilisation | Stock buffer (0.06M Tris-HCl, pH6.8, 1 mM EDTA, 2% | 0.59 |
| buffer | SDS) | |
| | Glycerol | 0.40 |
| | 2-mercapto-ethanol | 0.08 |
| | Saturated bromophenol blue | 0.04 |
| Separating | 30% Acrylamide stock (29.2% acrylamide and 0.8% | 9.30 |
| gel | bis-acrylamide (Bio-Rad Laboratories Ltd.)) | |
| | 9M Urea (BDH) | 6.40 |
| | 1.88M Tris/HCl, pH 8.8 | 4.00 |
| | 10% SDS | 0.20 |
| | Degas 10 min, then add | |
| | 10% Ammonium persulfate (Sigma) | 0.15 |
| | N,N,N′,N′-tetramethylethylenediamine (TEMED) (Sigma) | 0.01 |
| Stacking gel A | 30% Acrylamide stock (29.2% acrylamide and 0.8% | 1.00 |
| | bis-acrylamide) | |
| | 1.25M Tris/HCl, pH 6.8 | 1.00 |
| | 10% SDS | 0.10 |
| | DDW | 7.80 |
| | Degas 10 min, then add | |
| | 10% Ammonium persulfate | 0.10 |
| | TEMED | 0.01 |
| Stacking gel B | 30% Acrylamide stock (29.2% acrylamide and 0.8% | 1.00 |
| | bis-acrylamide) | |
| | 0.25M Tris/1.92 M glycine, pH 8.3 | 1.00 |
| | 10% SDS | 0.10 |
| | DDW | 7.80 |
| | Degas 10 min, then add | |
| | 10% Ammonium persulfate | 0.10 |
| | TEMED | 0.01 |

### Temperature Sensitivity:

Each of the 9 strains was inoculated onto two SBA plates. One of the two plates was aerobically incubated at 20° C. and the other at 42° C. The growth of each strain was examined at 48 h after inoculation.

### Hydrogen Sulphide Production:

Bacteria from a single colony of each strain on an SBA plate were inoculated onto an XLD plate and to TSI agar using conventional methods. They were then incubated at 37° C. for 48 hours. Strains that produced black pigment in TSI agar media and in the centre of isolated colonies on XLD media were identified as hydrogen sulphide producers.

### Lysine Decarboxylase (LD) Test:

Bacteria of each strain on an SBA plate were inoculated into LD broth medium and incubated at 37° C. for 48 hours. Strains that showed a change in colour of the media to dark purple were identified as producers of LD.

O-nitrophenyl-β-d-galactopyranoside (ONPG) Test:

Bacteria of each strain on an SBA plate were inoculated into an ONPG broth medium and incubated at 37° C. for 48 hours. Strains that grew in the media without colour change were determined to be lacking galactosidase.

### Fermentation of Carbohydrates:

Twenty different kinds of carbohydrate fermentation broth media were prepared following the procedure described previously. Carbohydrates used in this experiment were as follows:

### Adonitol [Sigma, A-5502]

D-(−)-Arabinose minimum 99% [Sigma, A-3131]
L-(+)-Arabinose minimum 99% [Sigma, A-3256]
D-(+)-Cellobiose minimum 98% [Sigma, C-7252]

### Dulcitol [Sigma, D-0256]

### D-(+)-Glucose 99.5% [Sigma, G-7528] (GC)

### Inositol (Meso) [BDH, 38044] (AR)

### Lactose [BDH, 10139] (AR)

Maltose [BDH, 29131] (Laboratory reagent (LR))

### Mannitol [Ajax, D-3247] (LR)

### D-(+)-Mannose [BDH, 01566]

### Melibiose [Sigma, M-5500]

### D-(+)-Raffinose [Sigma, R-0250]

### α-L-Rhamnose [Sigma, R-3875]

### Salicin [BDH, 38060]

D-Sorbitol minimum 98% [Sigma, S-1876]

### Sucrose 99+% [Sigma, S-0389] (AR)

### D-(+)-Trehalose [Sigma, T-5251]

### D-(+)-Xylose [Sigma, X-1500]

### L-(−)-Xylose [Sigma, X-1625]

To investigate gas production in a broth medium containing D-(+)-glucose, a small vial (5 mm×25 mm) was placed in an inverted position in a ¼ oz bottle in which 2 ml of the medium had been distributed. After autoclaving at 121° C. for 15 min, the vial was completely filled with the medium (Smibert and Krieg, General Characterization In Manual of Methods for General Bacteriology, p 415, Eds Gerhardt et al., American Society for Microbiology, Washington D.C., USA, 1981).

One individual colony of each of the 9 strains was inoculated into each broth medium containing a different carbohydrate. The media were then incubated at 37° C. for 10 days. The media were observed for red colour change and gas production every 24 h over the incubation period.

### Proteins:

Protein profiles of the strains were analysed using SDS-PAGE.

### Extraction of Whole Cell Proteins:

A fresh culture of each strain on SBA was harvested in 2 ml of 0.1M Tris/HCl (pH 6.8) containing 15% glycerol, 2 mM phenyl methyl-sulfonyl fluoride (PMSF) (BDH) and 2% SDS to obtain an approximate concentration of 10¹⁰ cfu/ml. The suspensions were boiled in a water bath for 5 min and whole cell protein extracts collected and stored at −20° C. until use.

### SDS-PAGE of Whole Cell Protein Extracts:

A 12% acrylamide separating gel and a 4% acrylamide stacking gel (Table 7) were used for the separation of bacterial proteins. Prior to running the gel, each sample was mixed with 2×SDS sample buffer (Table 7) at a ratio of 2:1. The mixture was then placed in boiling water for 5 min. Fifteen microliters of each mixture was loaded into the well. Electrophoresis was performed in a running buffer (0.025M Tris base/0.19M glycine (pH 8.3) containing 0.1% SDS) using a circulating cooling bath (at 10° C.) in a Mini Protean vertical electrophoresis unit (Bio-Rad Laboratories Ltd.) at 30 mA for 1 h. Following electrophoresis, the separating gel was stained with 0.1% Coomassie Brilliant Blue for an hour.

TABLE 8
COMPOSITION OF SEPARATING AND STACKING GELS
AND SAMPLE BUFFER.

| Solution | Component | Volume (ml) |
|---|---|---|
| Separating | 30% Acrylamide stock (29.2% acrylamide | 5.00 |
| gel | and 0.8% bis-acrylamide) | |
| | 1.5M Tris/HCl, pH 8.8 | 3.13 |
| | 10% SDS | 0.13 |
| | DDW | 4.18 |
| | Degas 10 min, then add | |
| | 10% Ammonium persulfate | 0.06 |
| | TEMED | 0.006 |
| Stacking gel | 30% Acrylamide stock (29.2% acrylamide | 0.65 |
| | and 0.8% bis-acrylamide) | |
| | 0.5M Tris/HCl, pH 6.8 | 1.25 |
| | 10% SDS | 0.05 |
| | DDW | 3.02 |
| | Degas 10 min, then add | |
| | 10% Ammonium persulfate | 0.03 |
| | TEMED | 0.005 |
| 2 × SDS | 0.5M Tris/HCl, pH 6.8 | 1.00 |
| sample buffer | Glycerol | 0.80 |
| | 10% SDS | 1.60 |
| | 2-mercapto-ethanol | 0.40 |
| | DDW | 0.40 |
| | Saturated bromophenol blue | 0.20 |

### Antimicrobial Sensitivity

### Antimicrobial Agents:

Commercial antimicrobial disks (Oxoid) were used for susceptibility tests. Antimicrobial agents and the amount contained in each disk are shown in Table 9.

TABLE 9
ANTIMICROBIAL AGENTS AND DISK CONTENTS USED IN
DISK SUSCEPTIBILITY TESTS.

| Group | Agent | Content (μg) |
|---|---|---|
| β-lactams penicillin | Amoxicillin | 10 |
| β-lactams/β-lactamase | Amoxicillin/Clavulanic acid | 20/10 |
| inhibitor combination | Ticarcillin/Clavulanic acid | 75/10 |
| Cephalosporin(1^{st}) | Cephalothin | 30 |
| Cephalosporin(3^{rd}) | Ceftazidime | 30 |
| Aminoglycoside | Gentamicin | 10 |
| Tetracycline | Tetracycline | 30 |
| Fluoroquinolones | Enrofloxacin | 5 |
| | Ciprofloxacin | 5 |
| Others | Sulphamethoxazole/Trimethoprim | 1.25/23.75 |

1^{st} = first generation;
3^{rd} = third generation.

### Antimicrobial Disk Susceptibility Tests:

The procedure employed in the tests was in accordance with the method proposed by The National Committee on Clinical Laboratory Standards (NCCLS) (Ferraro et al., Performance standards for antimicrobial susceptibility testing, NCCCLS 18:1-35, 1998; Watts et al., Performance standards for antimicrobial disk and dilution susceptibility test for bacteria isolated from animals, NCCLS 17:1-63, 1994). Briefly, MHA medium was dispensed into plastic culture plates with 9 cm internal diameters and flat bottoms to yield a uniform depth of 4 mm. These plates were kept at room temperature for 1 h and then dried in an incubator at 35° C. for 30 min. Four isolated colonies from a fresh culture of each strain were suspended in 4 ml of TSB and incubated at 37° C. for 3 h (for wild strain FD436) and 5 h (for mutant strains). PBS heated to 37° C. was then added to the suspensions to give approximately 10⁸ cfu/ml (comparable with the 0.5 McFarland turbidity standard). Each mixture was spread onto an MHA plate by streaking 3 times using a sterile cotton swab, and antimicrobial disks were applied to the plates with a dispenser (Oxoid). The inoculated plates were incubated at 37° C. for 18 h in an inverted position. Zone diameters for individual antimicrobials were translated in terms of susceptible, intermediate or resistant categories by referring to the zone diameter interpretative standards proposed by NCCLS (Ferraro et al., 1998, Supra; Watts et al., 1994, Supra).

### Results for Example 3

### Genotypic Characterization:

PCR amplification and nucleotide sequence of S. dublin gyrA genes:

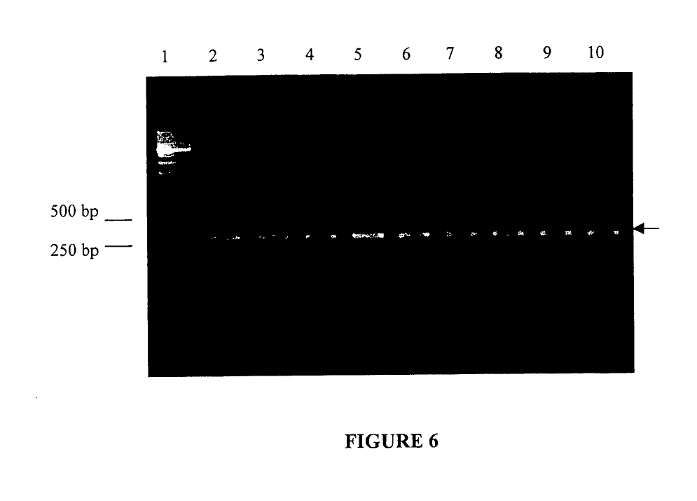

Based on the results of gyrA gene sequencing, the size of the amplified products of FD436 and all tested mutant strains was 347 bp (FIG. 6 and Table 10). A nucleotide substitution of A→G was identified in all tested N-RM strains. These substitutions resulted in an amino acid substitution of Asp-87→Gly. R-NM29 had a substitution of G→T, resulting in a Gly-81→cysteine (Cys) substitution. Sequencing repeated following 15 times subculture produced identical results.

PCR Amplification and Nucleotide Sequence of S. dublin rpoB Genes:

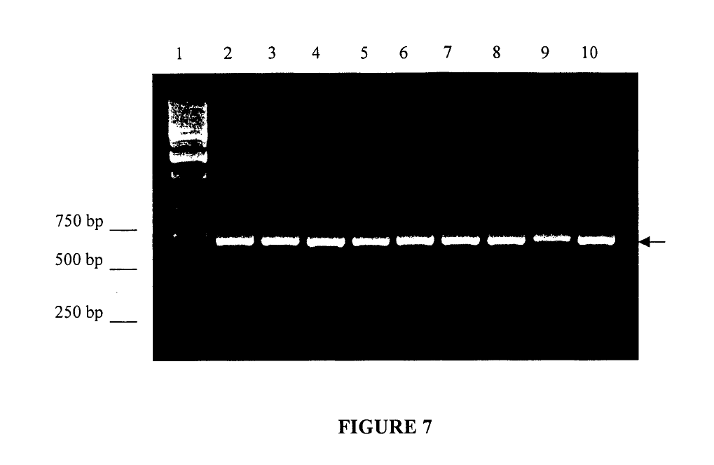

Based on the results of rpoB gene sequencing, products between the size of 696 and 714 bp were amplified from FD436 and the mutant strains (FIG. 7 and Table 11). N-RM4 had two point mutations, nucleotide substitutions of C→T and C→G, which resulted in amino acid substitutions of proline (Pro)-564→Leu and Gln-649→glutamic acid (Glu), respectively. N-RM15 showed a single point mutation, a nucleotide substitution of A→T, which caused an amino acid substitution of Ile-572→Phe. N-RM20 also had a nucleotide substitution of C→A that resulted in an amino acid substitution of Arg-529→Ser. Both N-RM25 and R-NM29 had a nucleotide substitution of A→G at the same position that resulted in an amino acid substitution of Asp-516→Gly. A deletion mutation of 12 nucleotides, TCTCAGTTTATG (SEQ ID NO:5), which resulted in a deletion of 4 amino acids, Ser-512, Gln-513, Phe-514 and methionine (Met)-515, was observed in both N-RM8 and N-RM9. N-RM27 showed an insertion mutation of 6 nucleotides, GACCAG (SEQ ID NO:6), which caused an insertion of 2 amino acids, Asp and Gln, between Met-515 and Asp-516. Pre- and post-subculture sequence results for each respective strain were identical.

TABLE 10
SEQUENCE ANALYSIS OF THE gyrA GENE OF S. dublin
DOUBLE ANTIMICROBIAL-RESISTANT STRAINS.

| Strain | Nucleotide change | Amino acid change |
|---|---|---|
| N-RM strains¹ | GAC → GGC | Asp-87 → Gly |
| R-NM29 | GGC → TGC | Gly-81 → Cys |

¹N-RM4, 8, 9, 15, 20 25 and 27; Asp = aspartic acid, Gly = glycine, Cys = cysteine.

TABLE 11
SEQUENCE ANALYSIS OF THE rpoB GENE OF S. dublin
DOUBLE ANTIMICROBIAL-RESISTANT STRAINS.

| Strain | Nucleotide change | Amino acid change |
|---|---|---|
| N-RM4 | CCG → CTG | Pro-564 → Leu |
| | CAG → GAG | Gln-649 → Glu |
| N-RM8 | Deletion of | Deletion of Ser-512, Gln-513, |
| | TCTCAGTTTATG | Phe-514, Met-515. |
| N-RM9 | Deletion of | Deletion of Ser-512, Gln-513, |
| | TCTCAGTTTATG | Phe-514, Met-515. |
| N-RM15 | ATC → TTC | Ile-572 → Phe |
| N-RM20 | CGT → AGT | Arg-529 → Ser |
| N-RM25 | GAC → GGC | Asp-516 → Gly |
| N-RM27 | Insertion of GACCAG | Insertion of Asp and Gln |
| | | between Met-515 and Asp-516 |
| R-NM29 | GAC → GGC | Asp-516 → Gly |

Arg = arginine,
Asp = aspartic acid,
Gln = glutamine,
Glu = glutamic acid,
Gly = glycine,
Ile = isoleucine,
Leu = leucine,
Met = methionine,
Phe = phenylalanine,
Pro = proline,
Ser = serine.

### Phenotypic Characterization

### Colony:

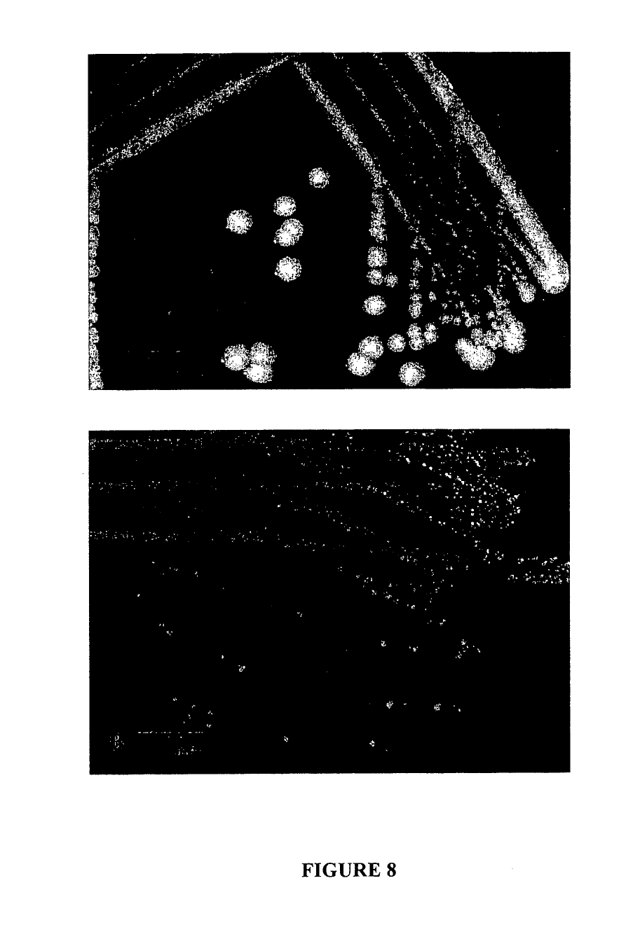

FD436 produced white-grey, opaque, circular, convex colonies with entire margins. The surface of the colonies was smooth and not haemolytic. The mutants, on the other hand, produced smaller colonies after 24-hour incubation than did FD436. Colonies of N-RM 20, 25 and 27 were particularly small. N-RM25 produced grey, translucent, slightly raised colonies that were easily distinguishable from colonies produced by FD436 and other tested mutants (FIG. 8). Details are shown in Table 12.

### Motility:

The results of the hanging drop method confirm that FD436 and all tested mutants were motile. Using the Craigie tube method, the time until the mutants were first isolated from the surface of the media outside the Craigie tubes was found to range from 12-33 h, longer than the time to first isolation for FD436 (9 h). No bacterial contamination of semi-solid media was evident during the Craigie tube test. Based on these results, motility was divided into three categories (time between inoculation and the first isolation: <12 h=high motility, 12 h-24 h=medium motility, >24 h=low motility). The results of this experiment are indicated in Table 13.

### Capsulation:

The wild strain was not capsulated, nor did any tested mutant strain possess a capsule.

### LPS:

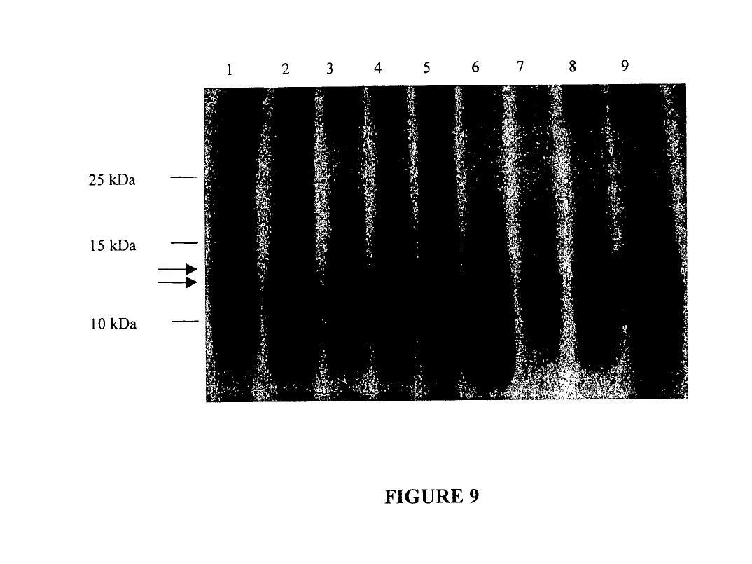

Regular ladder-like bands approximately 12-17 kDa in size (based on the migration of the protein marker) together with leading bands of approximately 8 kDa in size were observed in the LPS profiles of all strains tested except N-RM20. N-RM20 did not yield a regular banding pattern and the leading band of this strain migrated beyond 8 kDa (FIG. 9). No bands were observed on the gel stained with Coomassie Brilliant Blue except in the lane loaded with a protein marker.

### Growth:

### Temperature Sensitivity:

All tested strains grew on SBA media at both 20° C. and 42° C.

TABLE 12
COLONY MORPHOLOGY OF WILD STRAIN FD436 AND
MUTANTS N-RM4, 8, 9, 15, 20, 25, 27 AND R-NM29 POST 24-HOUR
INCUBATION AT 37° C. ON SBA.

| | | | Eleva- | | Colour & | |
|---|---|---|---|---|---|---|
| | Size¹ | Form | tion | Margin | opacity | Surface |
| FD436 | L | Circular | Convex | Entire | Grey & opaque | Glossy |
| N-RM4 | M | Circular | Convex | Entire | Grey & opaque | Glossy |
| N-RM8 | S | Circular | Convex | Entire | Grey & opaque | Glossy |
| N-RM9 | S | Circular | Convex | Entire | Grey & opaque | Glossy |
| N-RM15 | S | Circular | Convex | Entire | Grey & opaque | Glossy |
| N-RM20 | T | Circular | Convex | Entire | Grey & opaque | Glossy |
| N-RM25 | T | Circular | Raised | Entire | Grey & | Matt |
| | | | | | translucent | |
| N-RM27 | T | Circular | Convex | Entire | Grey & opaque | Glossy |
| R-NM29 | M | Circular | Convex | Entire | Grey & opaque | Glossy |

¹Average diameter of 5 isolated colonies of each strain. L = >2 mm; M = 1-2 mm; S = 0.5-1 mm; T = <0.5 mm.

TABLE 13
CHRONOLOGICAL OBSERVATION OF THE MOTILITY OF WILD
STRAIN FD436 AND MUTANTS N-RM4, 8, 9, 15, 20, 25, 27 AND
R-NM29 USING THE CRAIGIE TUBE METHOD.

| First isolation of Salmonella | | |
|---|---|---|
| post-inoculation (h) | Strain | Motility |
| 9 | FD436 | High |
| 12 | N-RM8,9,15 | High |
| 15 | R-NM29 | Medium |
| 18 | N-RM4 | Medium |
| 27 | N-RM20 | Low |
| 30 | N-RM25 | Low |
| 33 | N-RM27 | Low |

Time periods between inoculation and the first isolation of Salmonella strains from the surface of media outside the Craigie tubes: ≦12 h = high motility, 12 h-24 h = medium, ≧24 h = low.

### Biochemical Reactions

### Hydrogen Sulphide Production:

All tested strains produced black pigment both on XLD plates and in TSI agar slopes. Therefore, these strains were identified as hydrogen sulphide producers. However, although N-RM25 was similar to other tested strains in that it produced black pigment in a TSI agar slope within 24 h post-inoculation, production of hydrogen sulphide was markedly slower (taking almost 72 h) on XLD medium.

### LD Test:

All tested LD broth media turned yellow within a few hours post-inoculation and then turned dark purple. Therefore, the strains were identified as LD producers.

### ONPG Test:

At 48 hours following inoculation, no colour change in the ONPG broth media was observed, confirming that the tested strains lack galactosidase.

### Fermentation of Carbohydrates:

Variable results were obtained in D-(−)-arabinose and L-(+)-arabinose fermentation by FD436 and the mutants (Table 14), whereas fermentation of the other carbohydrates was consistent between all strains (Table 15). All tested N-RM strains were negative for gas production in the D-(+)-glucose fermentation test, whereas FD436 and R-NM29 were positive (Table 14).

TABLE 14
FERMENTATION OF D-(−)-ARABINOSE AND L-(+)-ARABINOSE
AND GAS PRODUCTION FROM D-(+)-GLUCOSE FERMENTATION
OF WILD STRAIN FD436 AND MUTANTS N-RM4, 8, 9, 15, 20, 25, 27
AND R-NM29.

| TEST | FD436 | 4 | 8 | 9 | 15 | 20 | 25 | 27 | 29 |
|---|---|---|---|---|---|---|---|---|---|
| D-(−)-arabinose | + | + | − | − | − | − | − | − | − |
| fermentation | | | | | | | | | |
| L-(+)-arabinose | + | − | − | − | + | − | − | − | + |
| fermentation | | | | | | | | | |
| Gas production | + | − | − | − | − | − | − | − | + |

+ = positive for fermentation or gas production,
− = negative for fermentation or gas production.

TABLE 15
CARBOHYDRATE FERMENTATION TESTS WITH WHICH THE
SAME RESULTS WERE OBTAINED FOR FD436,
N-RM4, 8, 9, 15, 20, 25, 27 AND R-NM29.

| Carbohydrate | Fermentation | Carbohydrate | Fermentation |
|---|---|---|---|
| Adonitol | − | Melibiose | + |
| D-(+)-cellobiose | − | D-(+)-raffinose | − |
| Dulcitol | + | α-L-rhamnose | + |
| D-(+)-glucose | + | Salicin | − |
| Inositol | − | D-sorbitol | + |
| Lactose | − | Sucrose | − |
| Maltose | + | D-(+)-trehalose | + |
| Mannitol | + | D-(+)-xylose | + |
| D-(+)-mannose | + | L-(−)-xylose | − |

+ = positive for fermentation,
− = negative for fermentation.

### Metabolism

### Proteins:

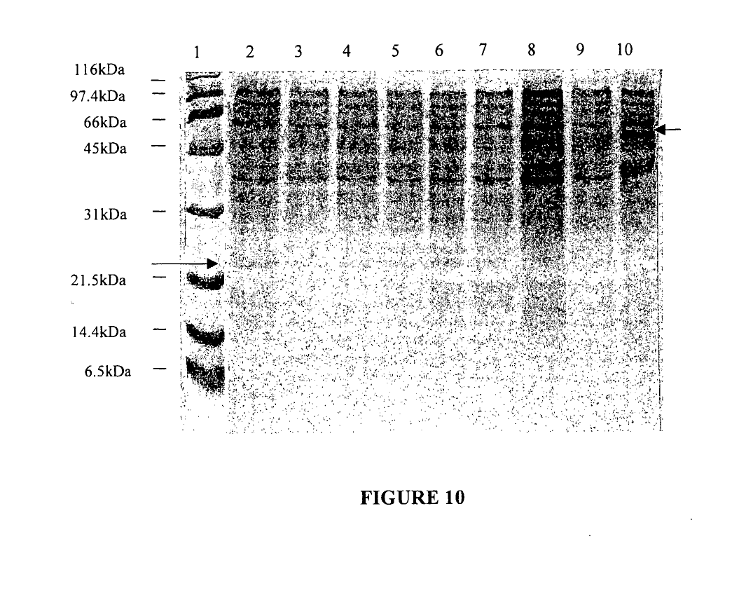

The estimated molecular weights of the major protein bands identified in SDS-PAGE for each strain ranged from approximately 18 kDa to 98 kDa (FIG. 10). There were 12 main protein bands with molecular weights of approximately 18, 20, 30, 35, 38, 41, 47, 53, 60, 70, 80 and 98 kDa that appeared to be shared among all tested strains. However, the 53 kDa band of R-NM29 was more intensely stained than that of wild strain FD436 and N-R mutants. In addition, although a band of approximately 24 kDa was observed in the protein profiles of the wild strain and the N-R mutants, no such band was detected in the protein profile of R-NM29.

### Antimicrobial Sensitivity:

FD436 and the mutants, except N-RM20, were susceptible to all antimicrobial agents used in the experiment. N-RM20 showed resistance to enrofloxacin.

### Example 4

### Bile Sensitivity as a Selection Marker for Vaccine Strains

### Experimental Procedures for Example 4:

### Bacteria:

Selected mutants N-RM4, N-RM25 and R-NM29 were prepared for the experiments following the procedure described previously. FD436 was also prepared as a control in the same manner.

### MIC AND MBC Determination

### Media:

Two millilitre aliquots of TSB media containing graded concentrations of BS No. 3 were prepared for the experiment. The concentrations of BS No. 3 used were 0.075, 0.15, 0.3, 0.6, 1.2, 2.4, 4.8, 9.6, 14.4, 19.2 and 24% (w/v). TSB media containing no BS No. 3 were also prepared as positive controls.

### Methods:

Approximately 10⁵ cfu/ml of each strain was suspended in TSB media containing different concentrations of BS No. 3 (the actual numbers of cells as determined by viable bacteria counts were FD436: 2.5×10⁵, N-RM4: 1.7×10⁵, N-RM25: 1.5×10⁵ and R-NM29: 2.0×10⁵ cfu/ml). Uninoculated media served as a negative control. For determining MICs, 200 μl of each medium was aseptically transferred to a sterile cuvette and the turbidity (OD₄₂₀₋₅₈₀) was measured at 37° C. for 24 h using Bioscreen. The MICs of BS No. 3 were determined based on the definition described in Section 3.2.7. The remainder of each medium was also incubated at 37° C. for 24 h and then used to determine MBC. To determine MBC, each medium containing greater than 2.4% BS No. 3 was diluted to a concentration of 2.4% by the addition of TSB. One millilitre of each medium was then pipetted directly onto an SBA plate and the medium distributed with a sterile glass spreader. These plates were incubated at 37° C. for 48 h. Colonies growing on the SBA were identified using the Salmonella identification procedure. The MBCs of BS No. 3 was determined based on the definition described previously.

### Growth I the Presence of Different Concentrations of Bile Salts

### Media:

TSB media containing graded concentrations of BS No. 3 were prepared for the experiment. The concentration of BS No. 3 in each medium was 0.075, 0.15, 0.3, 0.6, 1.2, 2.4, 4.8 and 9.6% (w/v). A TSB medium containing no BS No. 3 was also prepared as a positive control.

### Methods:

Turbidity of each strain in TSB containing different concentrations of BS No. 3 was determined at 37° C. for 20 h according to the procedure described previously. Turbidity of an uninoculated TSB medium was also measured as a negative control. The growth rate during the logarithmic growth phase of each strain was compared.

### Results for Example 4

### MIC and MBC Determination:

The MICs and MBCs of BS No. 3 for each strain are shown in Table 16.

TABLE 16
MICs AND MBCs OF BS NO. 3 FOR FD436 AND MUTANTS N-
RM4, N-RM25 AND R-NM29.

| Strain | MIC of BS No. 3 (%) | MBC of BS No. 3 (%) |
|---|---|---|
| FD436 | 19.2 | 24 |
| N-RM4 | 19.2 | 24 |
| N-RM25 | 14.4 | 24 |
| R-NM29 | 14.4 | 24 |

### Growth in the Presence of Different Concentrations of Bile Salts:

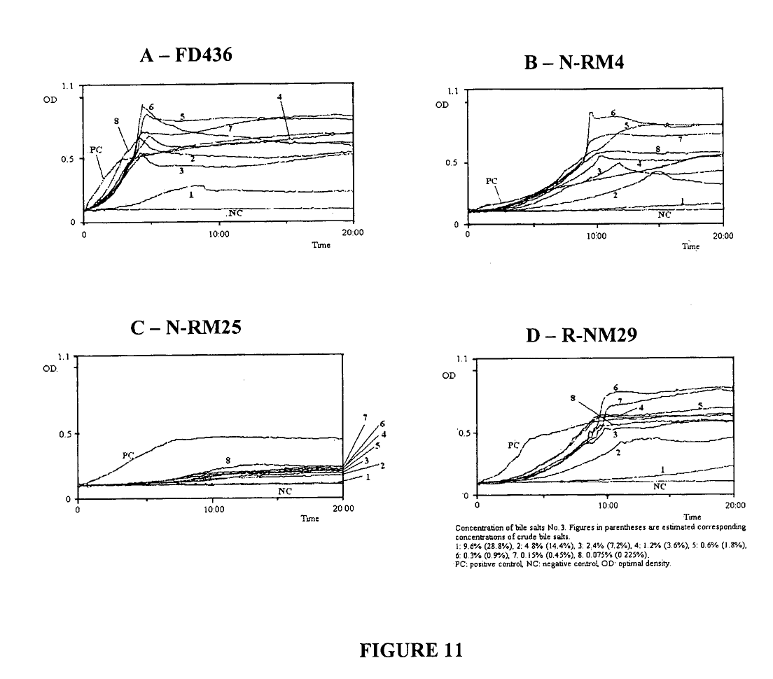

As shown in FIG. 11, all tested strains showed a prolonged lag phase in their growth cycles in the presence of BS No. 3, albeit to different degrees. Results from the twenty-hour incubation showed that, although the degree of inhibition differed for each strain, the higher the concentration of BS No. 3 the slower the growth rate. N-RM4 inoculated in TSB media containing less than 2.4% BS No. 3 produced faster growth than did the positive control after a prolonged lag phase. The growth of N-RM25 in TSB media containing even a very low concentration of BS No. 3 (as low as 0.075%) was much slower than the growth of this strain in a BS free medium. However, the growth of N-RM25 in graded concentrations of BS No. 3 was more uniform and consistent when compared with the other strains. In addition, the growth rate of N-RM25 was markedly slower when compared with the growth rates of other tested strains in TSB media containing the various concentrations of BS No. 3.

### Example 5

### Pathogenicity and Virulence of Selected Vaccine Strains in Mice

### Experimental Procedures for Example 5:

### Experimental Animals:

Mice were prepared following the procedure described previously.

### Challenge Strains:

FD436 and mutants N-RM4, N-RM25 and R-NM29 were prepared for the experiments following the procedure described previously. Challenge bacteria were prepared following the procedure described previously.

### ID₅₀ Determination

### Study Design:

Four groups of forty mice were each divided into eight groups, including a control group to determine the ID₅₀ of the bacterial strains (Table 17). Using PBS at 37° C., the prepared bacterial suspension of each strain was subjected to six serial ten-fold dilutions. For the challenge infection, 100 μl aliquots of each dilution, including the original, were administered to mice via intraperitoneal injection. Actual challenge doses are indicated in Table 17. One hundred microlitres of PBS containing no bacteria was administered to the negative control mice in the same manner. Clinical appearance of all mice was observed daily for 5 consecutive days post-inoculation (P.I.). An ID₅₀ for each strain was calculated based on the results of this five-day observation.

### Observation of Clinical Appearance:

Five parameters, activity, appetite, dehydration, respiratory rate and faecal consistency, were monitored daily (9:00 a.m.) in order to determine whether the mice suffered from salmonellosis caused by S. dublin. Mice showing clinical signs in three or more of the five parameters were classed as being infected by S. dublin and immediately euthanased. Heart blood, liver and spleen of the euthanased mice were removed immediately after euthanasia and investigated using the isolation and identification methods for Salmonella described previously.

TABLE 17
CHALLENGE DOSES OF FD436, N-RM4, N-RM25 AND R-NM29
FOR DETERMINING ID₅₀ FOR MICE.

| FD436 | | N-RM4 | | N-RM25 | | R-NM29 | |
|---|---|---|---|---|---|---|---|
| Group | Dose | Group | Dose | Group | Dose | Group | Dose |
| (n) | (cfu/mouse) | (n) | (cfu/mouse) | (n) | (cfu/mouse) | (n) | (cfu/mouse) |
| 1 (5) | 1.1 × 10⁸ | 1 (5) | 1.2 × 10⁸ | 1 (5) | 5.1 × 10⁸ | 1 (5) | 2.7 × 10⁸ |
| 2 (5) | 1.1 × 10⁷ | 2 (5) | 1.2 × 10⁷ | 2 (5) | 5.1 × 10⁷ | 2 (5) | 2.7 × 10⁷ |
| 3 (5) | 1.1 × 10⁶ | 3 (5) | 1.2 × 10⁶ | 3 (5) | 5.1 × 10⁶ | 3 (5) | 2.7 × 10⁶ |
| 4 (5) | 1.1 × 10⁵ | 4 (5) | 1.2 × 10⁵ | 4 (5) | 5.1 × 10⁵ | 4 (5) | 2.7 × 10⁵ |
| 5 (5) | 1.1 × 10⁴ | 5 (5) | 1.2 × 10⁴ | 5 (5) | 5.1 × 10⁴ | 5 (5) | 2.7 × 10⁴ |
| 6 (5) | 1.1 × 10³ | 6 (5) | 1.2 × 10³ | 6 (5) | 5.1 × 10³ | 6 (5) | 2.7 × 10³ |
| 7 (5) | 1.1 × 10² | 7 (5) | 1.2 × 10² | 7 (5) | 5.1 × 10² | 7 (5) | 2.7 × 10² |
| Control | 0 | Control | 0 | Control | 0 | Control | 0 |
| (5) | | (5) | | (5) | | (5) | |

n = number of mice in each grop;
cfu = colony forming unit.

### Penetration and Pathogenicity

### Study Design:

Fifty-four mice were divided into 4 groups and 4 mice placed in a negative control group (Table 18). Each mouse in all groups excluding the negative control group was experimentally infected once with 100 μl of a bacterial suspension containing FD436, N-RM 4, N-RM 25 or R-NM 29 intraperitoneally. The dose of each strain administered to the mice was approximately one-tenth of the ID₅₀ of the corresponding strain. One hundred microlitres of PBS containing no bacteria was administered to the negative control mice in the same manner. Actual doses are indicated in Table 18.

Clinical appearance was monitored daily for 9 consecutive days P.I. for the mice inoculated with FD436, N-RM4 and R-NM29 and 24 consecutive days P.I. for the mice inoculated with N-RM25 in accordance with the method described previously. To investigate faecal shedding of Salmonella, up to 10 faecal pellets were collected daily from each group. At set points in time during the experiments (Table 19), two mice in each group were fasted for 4 h, euthanased and immediately autopsied. Bacteriological investigation was conducted on heart blood, gall bladder bile and liver plus spleen samples collected from one of the euthanased mice in each group to evaluate the level of penetration by the inoculated organism. The gall bladder, liver and spleen of the second mouse in each group were subjected to histopathological examination.

Isolation of Salmonella from Faeces:

Isolation and identification methods described previously were employed to investigate whether or not Salmonella was excreted in faeces. Bacteria identified as being Salmonella were further examined to determine whether they were nalidixic acid and/or rifampicin resistant using the identification method described previously. As it was difficult to obtain a specific amount of faeces from each mouse every day, quantitative investigation was not undertaken.

TABLE 18
DOSES FOR EXPERIMENTAL INFECTION WITH FD436, N-RM4,
N-RM25 AND R-NM29.

| | | Number of mice in | Dose |
|---|---|---|---|
| Group | Strain | group | (cfu/mouse)* |
| 1 (Positive control) | FD436 | 10 | 4.9 × 10 |
| 2 | N-RM4 | 10 | 4.6 × 10³ |
| 3 | N-RM25 | 20 | 3.8 × 10⁷ |
| 4 | R-NM29 | 10 | 2.7 × 10⁷ |
| 5 (Negative control) | — | 4 | 0 |

*Bacteria in 100 μl of PBS were injected in each mouse by the intraperitoneal route. cfu = colony forming unit.

TABLE 19
TIMETABLE FOR EUTHANASIA.

| | Day | | | | | | | | | |
|---|---|---|---|---|---|---|---|---|---|---|
| Group | 1 | 3 | 5 | 7 | 9 | 12 | 15 | 18 | 21 | 24 |
| 1 | E | E | E | E | E | | | | | |
| 2 | E | E | E | E | E | | | | | |
| 3 | E | E | E | E | E | E | E | E | E | E |
| 4 | E | E | E | E | E | | | | | |
| 5 | E | | | | E | | | | | |

Day = number of days post-inoculation with S. dublin strains;
E = days on which two mice in each group were euthanased.

Isolation of Salmonella from Heart Blood and Gall Bladder Bile:

To detect the presence of Salmonella, approximately 20 μl of heart blood and 10 μl of gall bladder bile from one of the euthanased mice in each group were aseptically collected immediately after euthanasia using a 1 ml tuberculin syringe and a 26 G×½″ needle. Prior to the collection of heart blood, the surface of the heart was sterilised by cauterisation. The surface of the gall bladder was sterilised with 70% ethyl alcohol and then dried immediately before the collection of bile samples. Subsequent Salmonella isolation and identification procedures including nalidixic acid and/or rifampicin resistant strains were identical to those outlined previously. Similarly, because it was difficult to obtain a specific amount of heart blood or gall bladder bile from each mouse, quantitative investigation was not undertaken.

Quantitative and Qualitative Isolation of Salmonella from Liver Plus Spleen:

The liver plus spleen samples were placed in boiling water for 3 seconds to sterilise the surface of the organs before culturing. Samples from each mouse were weighed, homogenised together and mixed with PBS to make 5 ml suspensions. The enumeration method described previously was then used to determine the number of bacteria in each sample. The bacteria were subsequently identified as Salmonella or otherwise using the Salmonella identification method. Any samples that were Salmonella-negative in quantitative culturing were subjected to enrichment for qualitative Salmonella isolation using the EI methods described previously. Identification procedures for Salmonella and for nalidixic acid and/or rifampicin resistant Salmonella strains were identical to those outlined previously.

### Histopathological Investigation of Liver, Spleen and Gall Bladder:

Approximately 5 mm by 5 mm tissue samples from liver and spleen, and the entire gall bladder were removed from the mice and fixed in 10% formalin immediately after euthanasia. The tissue samples were processed by conventional means for histopathology. The sections were stained with hematoxylin and eosin (HE) and Giemsa stain, and examined to:
1. Assess lesions, suspected of being caused by inoculated organisms, in the liver, the spleen and the gall bladder and;
2. Evaluate the level of penetration of the gall bladder by the inoculated organisms.

To compare the degree of pathological changes between each inoculated strain, the results of these histopathological investigations were scored using the following scoring systems.

1. Histopathological (HP) score: Based on the level of inflammation, the histopathological changes in the liver spleen and gall bladder were individually scored on a scale of 0 to 3 (0=normal/no detectable change, 1=mild changes, 2=moderate changes, 3=marked changes—see Table 20 for descriptors of histopathological changes, see Table 27 for HP scores and Tables 28, 29 and 30 for details).
2. Bacterial penetration (BP) score: The levels of penetration of the gall bladder by the organisms were scored on a scale of 0 to 3 (0=no penetration, 1=penetration of the lumen only, 2=penetration of mucosa, 3=penetration of submucosa—see Table 27 for BP scores and Table 30 for details).

### Statistical Analysis:

The numbers of mice in each group confirmed as healthy and as ill due to salmonellosis were recorded each day throughout the observation periods. An analysis using Fisher's exact test (two-tailed) was performed on these data to determine whether there were significant differences in the health of mice in the groups when each strain was administered in doses of approximately ten times less than their respective ID₅₀.

TABLE 20
DESCRIPTORS OF HISTOPATHOLOGICAL CHANGES

| | Histopathological changes | |
|---|---|---|
| Degree | Liver & spleen | Gall bladder |
| Normal | Nil | Nil |
| Mild | A few suppurative foci and/or mild | One or more of the following changes: |
| | inflammatory infiltrate. | mild epithelial hyperplasia, mild oedema |
| | | of LP, margination of inflammatory |
| | | cells and mild inflammatory infiltrate. |
| Moderate | Moderate degree of one or more of the | Moderate degree of one or more of the |
| | above changes. | above changes. |
| Marked | Marked degree of one or more of the | Marked degree of one or more of the |
| | above changes and/or other severe | above changes and/or other severe |
| | changes such as vasculitis and | changes such as extension of |
| | coagulative necrosis. | inflammation to subserosa. |

LP = Lamina propria.

### Results for Example 5

### ID₅₀ Determination:

After challenge infection, mice suffering from salmonellosis were found in all groups inoculated with FD436 or N-RM4. Clinical signs shown by mice in these groups included low activity, low appetite, dehydration and high respiratory rates, but not abnormal faecal consistency. None of the mice administered N-RM25 or R-NM29 showed signs of salmonellosis throughout the observation period. Based on the results of clinical observations, the ID₅₀ for each strain was calculated to be 7.3×10² cfu for FD436, 6.9×10³ cfu for N-RM4, >5.1×08 cfu for N-RM25 and >2.7×10⁸ cfu for R-NM29. Details are shown in Tables 21-24.

TABLE 21
FIVE-DAY OBSERVATION OF CLINICAL APPEARANCE
OF MICE FOLLOWING EXPERIMENTAL INFECTION WITH
DIFFERENT DOSES OF FD436.

| | Number of mice | | | | |
|---|---|---|---|---|---|
| Group¹ | determined as being infected by S. dublin | | | | |
| (dose)² | Day³ 1 | Day 2 | Day 3 | Day 4 | Day 5 |
| 1 (1.1 × 10⁸) | 0 | 0 | 5 | — | — |
| 2 (1.1 × 10⁷) | 0 | 0 | 5 | — | — |
| 3 (1.1 × 10⁶) | 0 | 0 | 2 | 3 | — |
| 4 (1.1 × 10⁵) | 0 | 0 | 2 | 3 | — |
| 5 (1.1 × 10⁴) | 0 | 0 | 0 | 2 | 1 |
| 6 (1.1 × 10³) | 0 | 0 | 0 | 1 | 2 |
| 7 (1.1 × 10²) | 0 | 0 | 0 | 0 | 2 |
| Control (0) | 0 | 0 | 0 | 0 | 0 |

¹The number of mice in each group was 5;
²cfu;
³number of days post-inoculation with the corresponding strains.

TABLE 22
FIVE-DAY OBSERVATION OF CLINICAL APPEARANCE
OF MICE FOLLOWING EXPERIMENTAL INFECTION WITH
DIFFERENT DOSES OF N-RM4.

| | Number of mice determined as being | | | | |
|---|---|---|---|---|---|
| Group¹ | infected by S. dublin | | | | |
| (dose)² | Day³ 1 | Day 2 | Day 3 | Day 4 | Day 5 |
| 1 (1.2 × 10⁸) | 0 | 0 | 1 | 4 | — |
| 2 (1.2 × 10⁷) | 0 | 0 | 0 | 3 | 2 |
| 3 (1.2 × 10⁶) | 0 | 0 | 0 | 2 | 3 |
| 4 (1.2 × 10⁵) | 0 | 0 | 0 | 1 | 3 |
| 5 (1.2 × 10⁴) | 0 | 0 | 0 | 0 | 2 |
| 6 (1.2 × 10³) | 0 | 0 | 0 | 0 | 2 |
| 7 (1.2 × 10²) | 0 | 0 | 0 | 0 | 1 |
| Control (0) | 0 | 0 | 0 | 0 | 0 |

¹The number of mice in each group was 5;
²cfu;
³number of days post-inoculation with the corresponding strains.

TABLE 23
FIVE-DAY OBSERVATION OF CLINICAL APPEARANCE
OF MICE FOLLOWING EXPERIMENTAL INFECTION WITH
DIFFERENT DOSES OF N-RM25.

| | Number of mice determined as being | | | | |
|---|---|---|---|---|---|
| Group¹ | infected by S. dublin | | | | |
| (dose)² | Day³ 1 | Day 2 | Day 3 | Day 4 | Day 5 |
| 1 (5.1 × 10⁸) | 0 | 0 | 0 | 0 | 0 |
| 2 (5.1 × 10⁷) | 0 | 0 | 0 | 0 | 0 |
| 3 (5.1 × 10⁶) | 0 | 0 | 0 | 0 | 0 |
| 4 (5.1 × 10⁵) | 0 | 0 | 0 | 0 | 0 |
| 5 (5.1 × 10⁴) | 0 | 0 | 0 | 0 | 0 |
| 6 (5.1 × 10³) | 0 | 0 | 0 | 0 | 0 |
| 7 (5.1 × 10²) | 0 | 0 | 0 | 0 | 0 |
| Control (0) | 0 | 0 | 0 | 0 | 0 |

¹The number of mice in each group was 5;
²cfu;
³number of days post-inoculation with the corresponding strains.

TABLE 24
FIVE-DAY OBSERVATION OF CLINICAL APPEARANCE
OF MICE FOLLOWING EXPERIMENTAL INFECTION WITH
DIFFERENT DOSES OF N-RM29.

| | Number of mice | | | | |
|---|---|---|---|---|---|
| Group¹ | determined as being infected by S. dublin | | | | |
| (dose)² | Day³ 1 | Day 2 | Day 3 | Day 4 | Day 5 |
| 1 (2.7 × 10⁸) | 0 | 0 | 0 | 0 | 0 |
| 2 (2.7 × 10⁷) | 0 | 0 | 0 | 0 | 0 |
| 3 (2.7 × 10⁶) | 0 | 0 | 0 | 0 | 0 |
| 4 (2.7 × 10⁵) | 0 | 0 | 0 | 0 | 0 |
| 5 (2.7 × 10⁴) | 0 | 0 | 0 | 0 | 0 |
| 6 (2.7 × 10³) | 0 | 0 | 0 | 0 | 0 |
| 7 (2.7 × 10²) | 0 | 0 | 0 | 0 | 0 |
| Control (0) | 0 | 0 | 0 | 0 | 0 |

¹The number of mice in each group was 5;
²cfu;
³number of days post-inoculation with the corresponding strains.

### Penetration and Pathogenicity

### Observation of Clinical Appearance:

As indicated in Table 25, in Group I (positive control inoculated with FD436), two mice showed clinical signs of salmonellosis on Day 3 P.I. By Day 5, all surviving mice (6 in total) became ill and two died on that day. The remaining mice died the next day. All mice remaining in Group 2 (inoculated with N-RM4) showed clinical signs of salmonellosis on Day 4. This condition continued and one mouse died on Day 7. The last mice in Group 2 died on Day 8. Clinical signs shown by mice in these groups included low activity, low appetite, dehydration and high respiratory rates. Meanwhilst, all mice in Group 3 (inoculated with N-RM25), Group 4 (inoculated with R-NM29) and Group 5 (negative control) showed no signs of illness throughout the observation period, except 2 mice in group 4 which were ill on Day 5 and then recovered. Statistics show that compared with mice in Groups 1 and 2, mice in Groups 3, 4 and 5 were significantly healthier (between Group 1 and Group 3: p<0.0001, Group 1 and Group 4: p<0.001, Group 1 and Group 5: p<0.005, Group 2 and Group 3: p<0.0001, Group 2 and Group 4: p<0.0001, Group 2 and Group 5: p<0.0005). There was no significant difference in the health of mice between Group 1 and Group 2, nor between Groups 3, 4 and 5.

Isolation of Salmonella from Faeces:

Mice in Groups 1 and 2 first excreted Salmonella in their faeces on Day 3 and Day 4 P.I., respectively. Salmonella were consistently isolated using the DI method until all mice in Groups 1 and 2 were euthanased or died. Throughout the twenty-four day observation period, Salmonella was isolated from the faeces of mice in Group 3 intermittently between Day 5 and Day 12, and only using the EI method. On all other days, the mice were culture negative. Salmonella was not isolated from the faeces of mice in Groups 4 and 5 during the nine-day observation period. All Salmonella isolates from mice in Groups 2 and 3 were nalidixic acid and rifampicin resistant (Table 25).

TABLE 25
CHRONOLOGICAL INVESTIGATION OF CLINICAL
APPEARANCE OF AND Salmonella FAECAL EXCRETION BY MICE IN
GROUPS INOCULATED WITH FD436, N-RM4, N-RM25 OR R-NM29 VIA THE
INTRAPERITONEAL ROUTE.

| | | Day² | | | | | | | | |
|---|---|---|---|---|---|---|---|---|---|---|
| Group¹ | | 1-3 | 4-6 | 7-9 | 10-12 | 13-15 | 16-18 | 19-21 | 22-24 | Total |
| Total of ill mice/Total | | 2/26 | 10/10 | ND⁵ | | | | | | 12/36 |
| of surviving mice³ | | | (4)⁶ | | | | | | | |
| Faecal | DI | 1S/3 | 2S/2 | ND | | | | | | 3S/5 |
| excretion⁴ | EI | 1S/3 | 2S/2 | ND | | | | | | 3S/5 |
| Total of ill mice/Total | | 0/26 | 16/16 | 3/3 | | | | | | 9/45 |
| of surviving mice | | | | (2)⁷ | | | | | | |
| Faecal | DI | 0/3 | 3R/3 | 1R/1 | | | | | | 4R/7 |
| excretion | EI | 0/3 | 3R/3 | 1R/1 | | | | | | 4R/7 |
| Total of ill mice/Total | | 0/56 | 0/46 | 0/38 | 0/30 | 0/24 | 0/18 | 0/12 | 0/6 | 0/230* |
| of surviving mice | | | | | | | | | | |
| Faecal | DI | 0/3 | 0/3 | 0/3 | 0/3 | 0/3 | 0/3 | 0/3 | 0/3 | 0/24 |
| excretion | EI | 0/3 | 2R/3 | 2R/3 | 2R/3 | 0/3 | 0/3 | 0/3 | 0/3 | 6R/24 |
| Total of ill mice/Total | | 0/26 | 2/16 | 0/8 | | | | | | 2/50* |
| of surviving mice | | | | | | | | | | |
| Faecal | DI | 0/3 | 0/3 | 0/3 | | | | | | 0/9 |
| excretion | EI | 0/3 | 0/3 | 0/3 | | | | | | 0/9 |
| Total of ill mice/Total | | 0/8 | 0/6 | 0/6 | | | | | | 0/20* |
| of surviving mice | | | | | | | | | | |
| Faecal | DI | 0/3 | 0/3 | 0/3 | | | | | | 0/9 |
| excretion | EI | 0/3 | 0/3 | 0/3 | | | | | | 0/9 |

¹Salmonella strains inoculated to mice were: Group 1 (positive control) - FD436; Group 2 - N-RM4; Group 3 - N-RM25; Group 4 - R-NM29; Group 5 - negative control. Refer to Table 18 for infectious doses.
²Day post-inoculation.
³Two mice in each group were euthanased following the timetable indicated in Table 19.
⁴Results represent the number of culture positive for Salmonella over the total number of samples tested in each group. DI: Salmonella was isolated using the direct isolation method, EI: Salmonella was isolated using the enrichment isolation method, S: isolated Salmonella was sensitive to nalidixic acid and rifampicin, R: isolated Salmonella was resistant to nalidixic acid and rifampicin.
⁵No data were available as no mice survived after Day 6.
⁶&⁷Numbers in parentheses indicate the number of mice which died during the periods in question (⁶two mice died on both Days 5 and 6 P.I., ⁷: one mouse died on both Days 7 and 8 P.I.).
*In comparison with Group 1 (positive control), Group 3 p < 0.0001, Group 4: p = 0.0006, Group 5: p = 0.0046 (Fisher's exact test (two-tailed)).

Isolation of Salmonella from Heart Blood:

As shown in Table 26, Salmonella was first isolated from heart blood of euthanased mice in all groups, except Group 5, on the first day following inoculation. The organism was isolated from all heart blood samples of mice in Groups 1 and 2 using the DI method until all mice in these groups were euthanased or died. The organism was isolated on Day 3 P.I. from heart blood samples of mice in Group 3 with the DI method. From this point on, the organism was only isolated from a heart blood sample of one mouse in Group 3 using the EI method on Day 7. The organism was not isolated from heart blood samples of mice in Group 4 during the nine-day observation period except on Day 1. No bacteria were isolated from the heart blood samples of mice in Group 5.

Isolation of Salmonella from Gall Bladder Bile:

Salmonella was first isolated from gall bladder bile samples of mice in Groups 1, 2 and 3 using the DI method on Day 5 P.I. Afterwards, the organism was isolated from gall bladder bile samples of mice in Group 2 through the DI method on Day 7 (by which time all mice in this group were euthanased or had died). During the twenty-four day observation period, the organism was also isolated from gall bladder bile of mice in Group 3 through the DI method on Day 12 and Day 15. Salmonella was also isolated on Day 21 P.I. but only when the EI method was used. The organism was not isolated from gall bladder bile of mice in Groups 4 and 5 (Table 26).

Quantitative and Qualitative Isolation of Salmonella from Liver Plus Spleen:

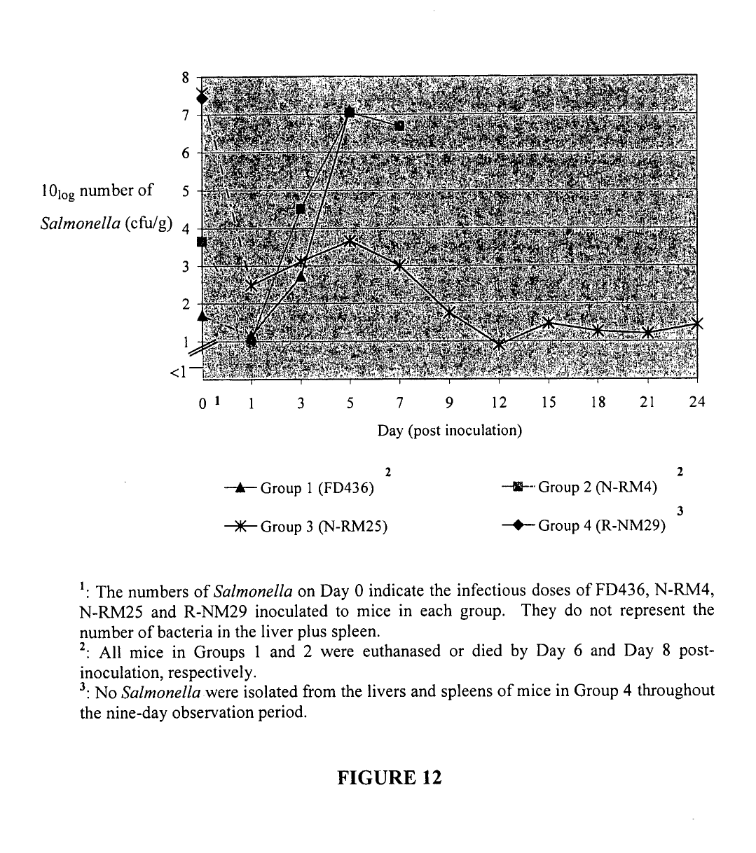

Salmonella was isolated quantitatively from the livers and spleens of mice in Groups 1, 2 and 3 continuously throughout the observation period (FIG. 12). The number of Salmonella isolated from the organs of mice in Groups 1 and 2 increased from approximately 10 to approximately 10⁷ cfu per gram within 5 days P.I. In contrast, although the dose of N-RM25 administered to mice in Group 3 was approximately 10⁴ to 10⁶ times greater than that of FD436 and N-RM4, the number of Salmonella isolated from the livers and spleens of mice in Group 3 decreased from approximately 10³ to approximately 10 cfu per gram by Day 12 P.I. The number of Salmonella in the livers and spleens of mice in Group 3 then remained stable at around 10 cfu per gram between Day 12 and Day 24. No bacteria, including Salmonella, were isolated from the livers and spleens of mice in Groups 4 and 5 using either direct or enrichment methods (Table 17).

TABLE 26
CHRONOLOGICAL INVESTIGATION OF THE ISOLATION OF
Salmonella FROM HEART BLOOD, GALL BLADDER BILE AND
LIVER PLUS SPLEEN OF MICE INOCULATED WITH FD436,
N-RM4, N-RM25 OR R-NM29 VIA THE INTRAPERITONEAL
ROUTE.

| GROUP² | | Day¹ | | | | | | | | | |
|---|---|---|---|---|---|---|---|---|---|---|---|
| SAMPLE³ | | 1 | 3 | 5 | 7 | 9 | 12 | 15 | 18 | 21 | 24 |
| 1 | HB⁴ | +S | +S | +S | ND | ND | | | | | |
| | GB | − | − | +S | ND | ND | | | | | |
| | L&S | +S | +S | +S | ND | ND | | | | | |
| 2 | HB | +R | +R | +R | +R | ND | | | | | |
| | GB | − | − | +R | +R | ND | | | | | |
| | L&S | +R | +R | +R | +R | ND | | | | | |
| 3 | HB | +R | +R | − | +RE | − | − | − | − | − | − |
| | GB | − | − | +R | − | − | +R | +R | − | +RE | − |
| | L&S | +R | +R | +R | +R | +R | +R | +R | +R | +R | +R |
| 4 | HB | +R | − | − | − | − | | | | | |
| | GB | − | − | − | − | − | | | | | |
| | L&S | − | − | − | − | − | | | | | |
| 5 | HB | − | ND | ND | ND | − | | | | | |
| | GB | − | ND | ND | ND | − | | | | | |
| | L&S | − | ND | ND | ND | − | | | | | |

¹Day post-inoculation.
²Salmonella strains inoculated to mice were: Group 1 (positive control) - FD436; Group 2 - N-RM4; Group 3 - N-RM25, Group 4 - R-NM29; Group 5 - negative control. Refer to Table 18 for infectious doses.
³HB - heart blood, GB - gall bladder bile, L&S - liver plus spleen.
⁴+: positive for Salmonella isolation by using direct isolation method, −: negative for Salmonella isolation, S: Salmonella isolated were sensitive to nalidixic acid and rifampicin, R: Salmonella isolated were resistant to nalidixic acid and rifampicin, E: positive for Salmonella isolation only by using enrichment isolation method, ND: no data available as mice either did not survive or were not euthanased.

### Autopsy:

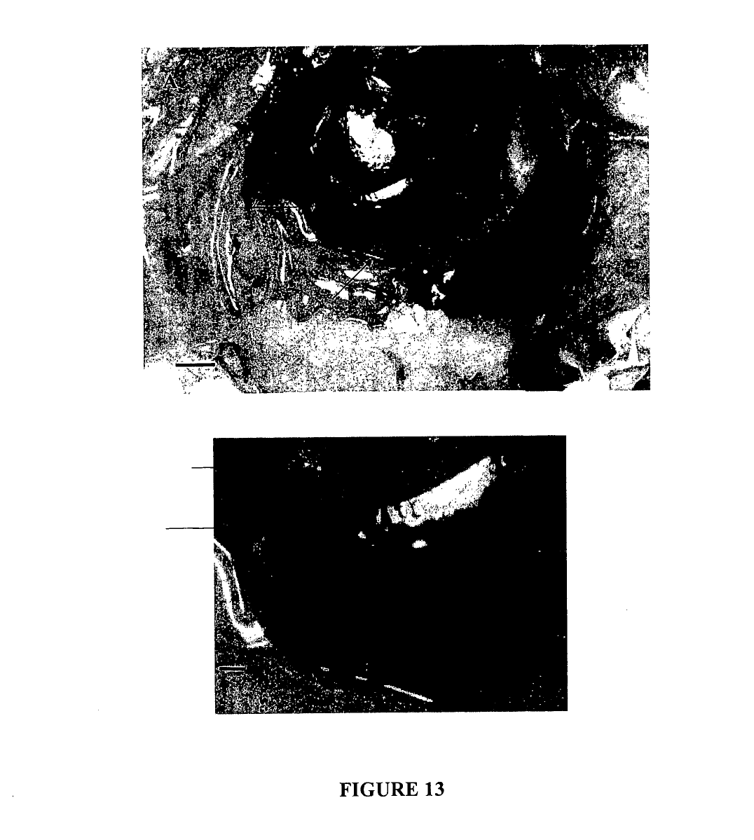

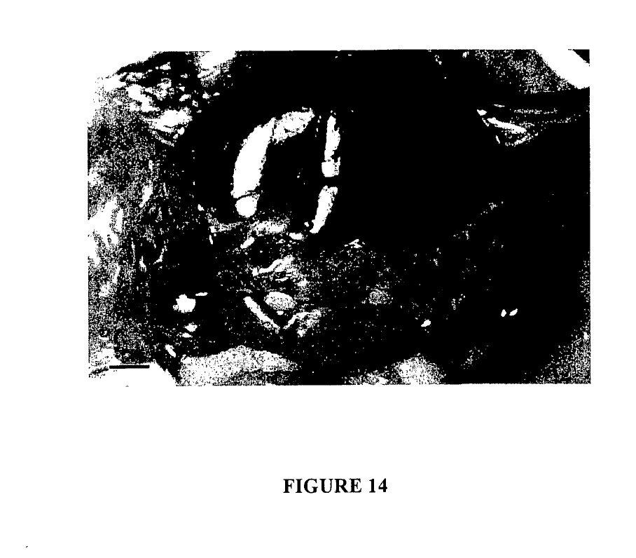

No gross changes were evident in the organs of mice euthanased on the day following inoculation. In Group 1, scattered petechial haemorrhages were observed on the serosa of the liver in mice euthanased on Day 3 P.I. Cloudy, straw-coloured fibrinous exudate appeared on the surface of the serosa of the visceral organs and the peritoneum of mice in Group 1 euthanased on Day 5. Splenomegaly and petechial haemorrhages on the serosa of the visceral organs (especially the liver) were also prominent in the same mice. In mice in Group 2 euthanased on Day 5, petechial haemorrhages scattered on the entire surface of the liver and the intestine and splenomegaly were observed. One of the mice also showed marked fibrinous exudate in the abdominal cavity. Prominent splenomegaly, petechial haemorrhages on the serosa of the visceral organs and fibrinous exudate on the surface of the visceral organs and peritoneum were observed in mice in Group 2 euthanased on Day 7. There were small (up to miliary size) grey-white foci scattered across the entire surface of the liver of one of these mice (FIGS. 13A and 13B). In contrast to these findings, no gross lesions were observed upon autopsy of the mice in Group 3 throughout the observation period with the exception of slight to moderate splenomegaly in some mice euthanased on Days 5, 7, 9 and 12 (FIG. 14). No lesions were observed in visceral organs in mice in Groups 4 and 5 throughout the observation period.

### Histopathological Investigation of Liver, Spleen and Gall Bladder

### Group 1:

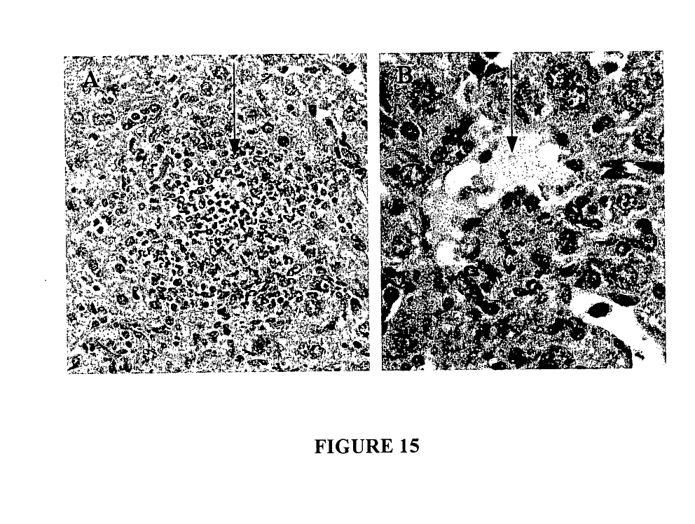

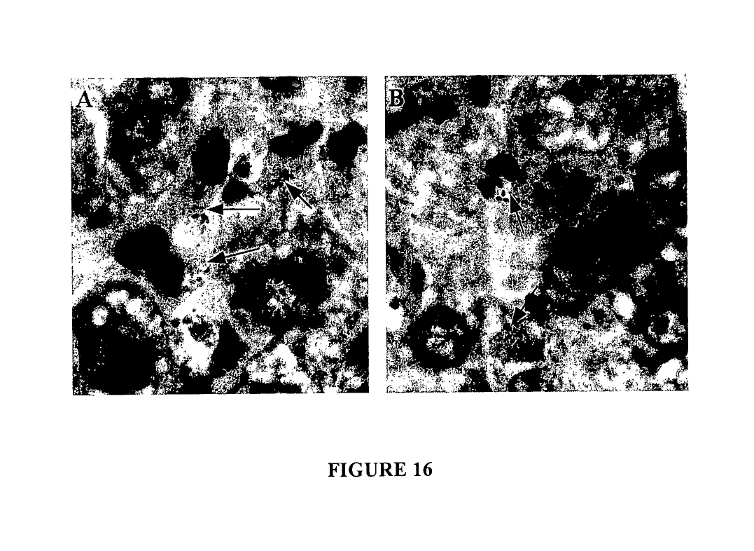

A few foci of mild inflammatory infiltrate characterised by neutrophils and lymphocytes were observed in the liver of the mouse euthanased on the day following inoculation. In this organ, there was widespread infiltration by PMNs and randomly distributed foci of lytic necrosis with suppuration on Day 3 P.I. (FIG. 15) followed by an increase in infiltration of the sites by macrophages on Day 5. Vasculitis of portal vessels and coagulative necrosis were also evident. Putative Salmonella cells were observed predominantly in Kupffer cells on Day 5 (FIG. 16). All mice in this group surviving until Day 5 subsequently died the following day.

Compared with the liver samples, the progress of inflammation in the spleen of mice in this group was somewhat slower. A moderate inflammation in the sinus areas associated with sinusoidal hypercellularity was found on Day 3 P.I. There was a prominent increase in the infiltration by PMNs to these sites on Day 5.

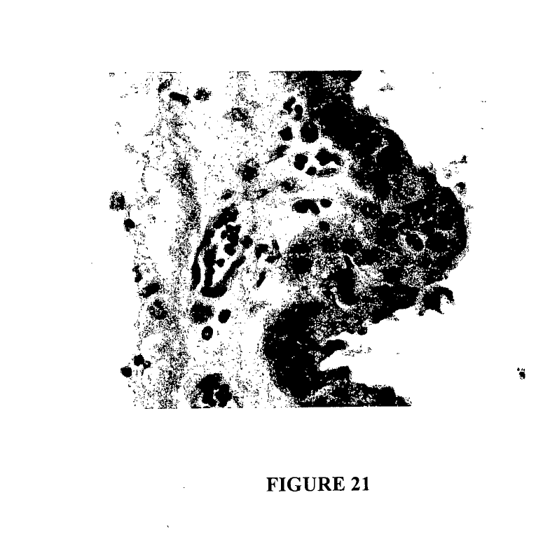

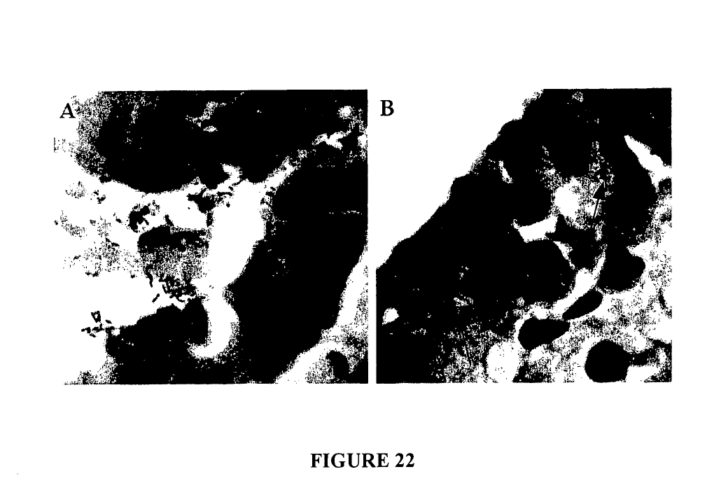

In the gall bladder, obvious changes in histopathology were not observed until Day 3 P.I. Marked epithelial hyperplasia, infiltration by lymphocytes and monocytes, and oedema of the lamina propria were evident on Day 5 (FIG. 21). Prominent penetration by bacteria of the lumen, mucosa and submucosa in the gall bladder was also observed on the same day (FIG. 22).

### Group 2:

As observed in Group 1, one mouse in this group euthanased on Day 3 also showed marked histopathological changes in the liver, such as randomly distributed foci characterised by active infiltration by PMNs. Vasculitis of portal vessels and coagulative necrosis were prominent and putative Salmonella cells were predominantly observed in Kupffer cells on Day 5 (FIG. 16). A change in predominant cell types from PMNs to lymphocytes and plasma cells between Day 5 and Day 7 was evident. All mice in this group died by Day 8.

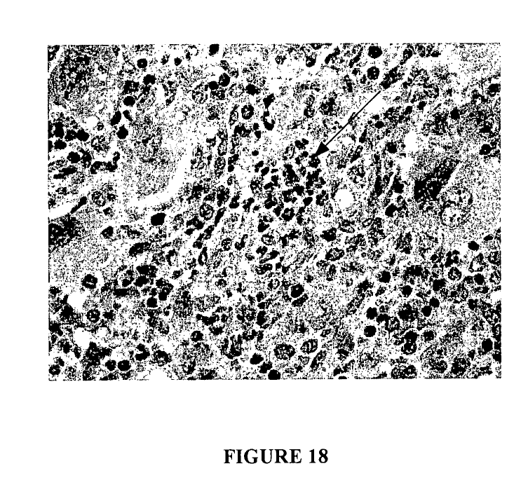

Acute splenitis was evident from the active infiltration by PMNs in the spleen of a mouse euthanased on Day 3 (FIG. 18). Lytic necrosis in sinus areas and vasculitis accompanied by vascular thrombosis were prominent on Day 5. A change in the types of leukocyte in the sites from PMNs to mononuclear (MN) cells, such as macrophages occurred on Day 7.

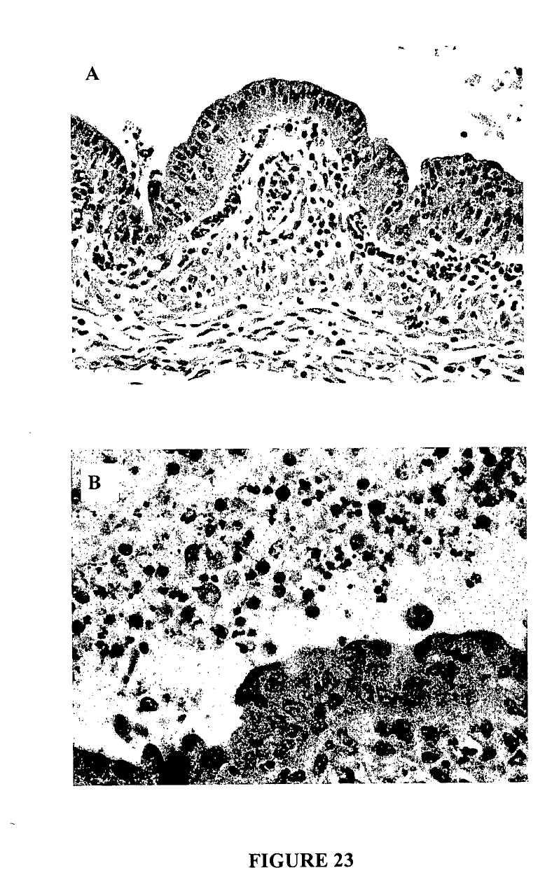

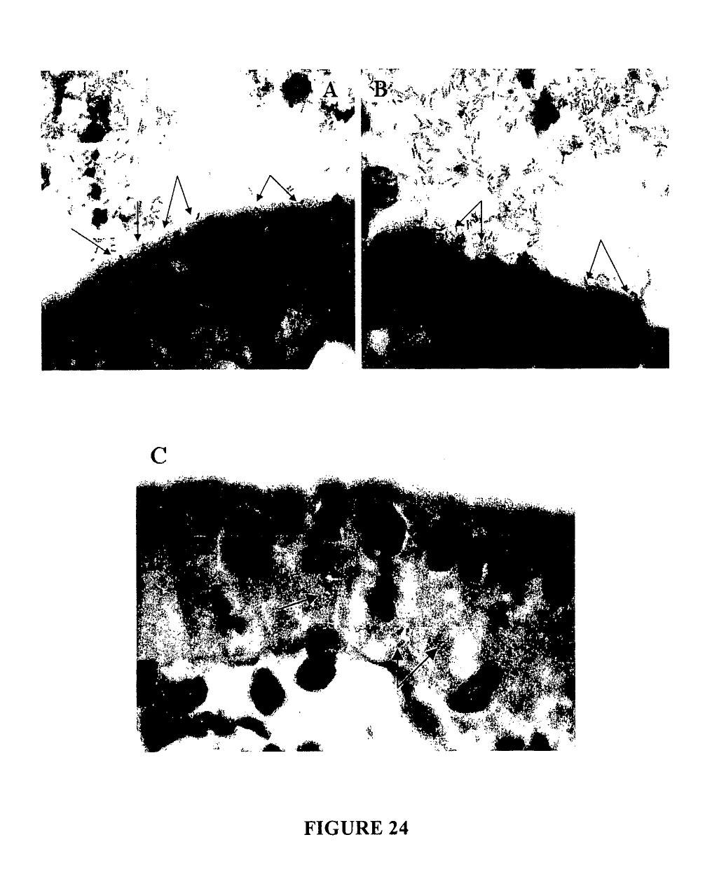

In the gall bladder, mild infiltration of the lamina propria by PMNs and MN cells, and bacterial penetration of the lumen were observed on Day 5. The extension of inflammation from the mucosa to subserosa and the progression to severe acute cholecystitis were evident in a mouse euthanased on Day 7. This was characterised by prominent infiltration by PMNs throughout the gall bladder wall, marked epithelial hyperplasia, haemorrhage and fibroplasia in the lamina propria, and subserosal oedema and fibroplasia (FIG. 23A). Marked intraluminal inflammatory exudate by PMNs, monocytes and lymphocytes was also seen (FIG. 23B). Numerous bacteria were present in the lumen and adhering to the brush border. Some organisms were also observed in the epithelial cells and submucosa (FIG. 24).

### Group 3:

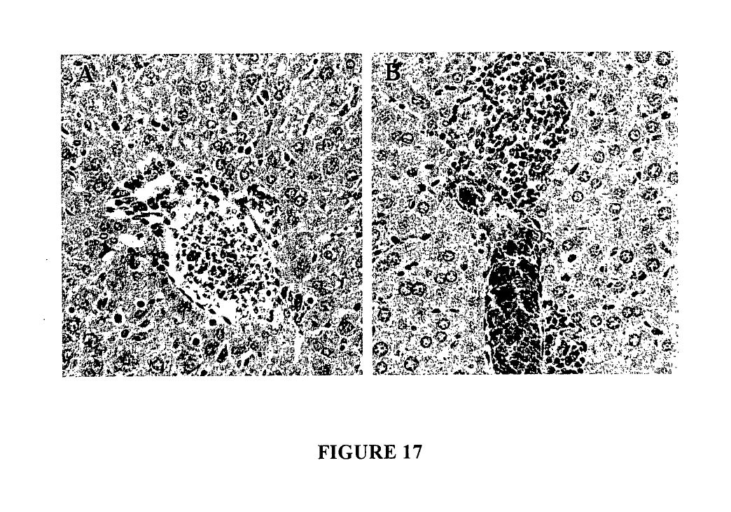

Compared with the above two groups, histopathological changes in the liver of mice in this group throughout the observation period were mild. Mild inflammation, such as periportal infiltration by MN cells and a few to a moderate number of foci of neutrophils, was seen between Day 3 and Day 12. During the same period, the number of suppurative foci markedly decreased (mainly PMNs were present in the early stages, after which they were gradually replaced by lymphocytes). Mild inflammation characterised by periportal inflammatory infiltrate and occasional foci continued throughout the twenty-four day observation period (FIG. 17). Differing from Groups 1 and 2, prominent hepatocellular regeneration was evident in the liver of mice in this Group euthanased after Day 7 P.I.

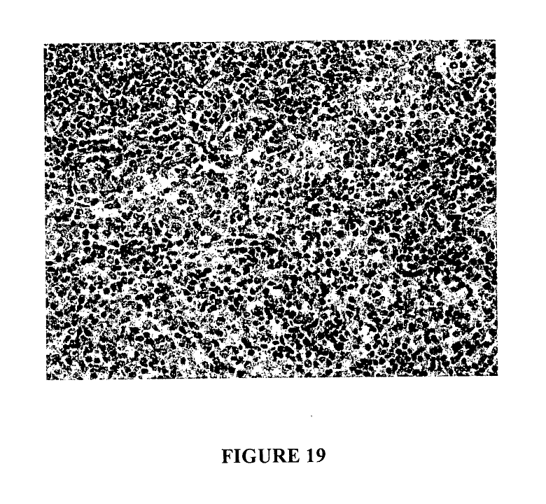

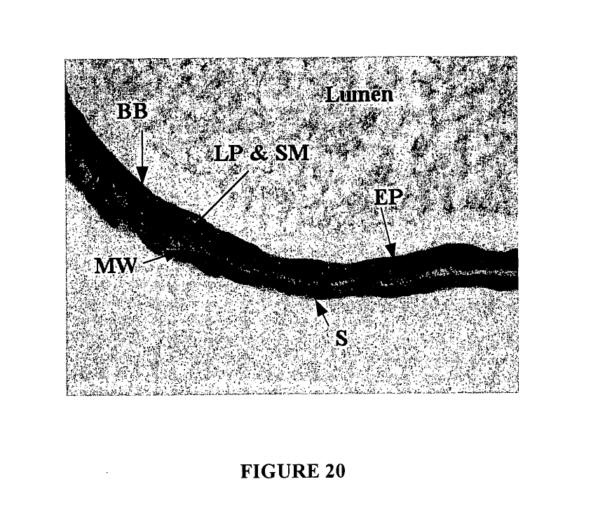

In the spleen, mild inflammation characterised by lymphoid hyperplasia, some foci with suppuration in sinus areas (although fewer in number than in mice in Groups 1 and 2) and extramedullary haematopoiesis were observed between Day 5 and Day 9. Apparent inflammation was no longer evident after Day 12 (FIG. 19).

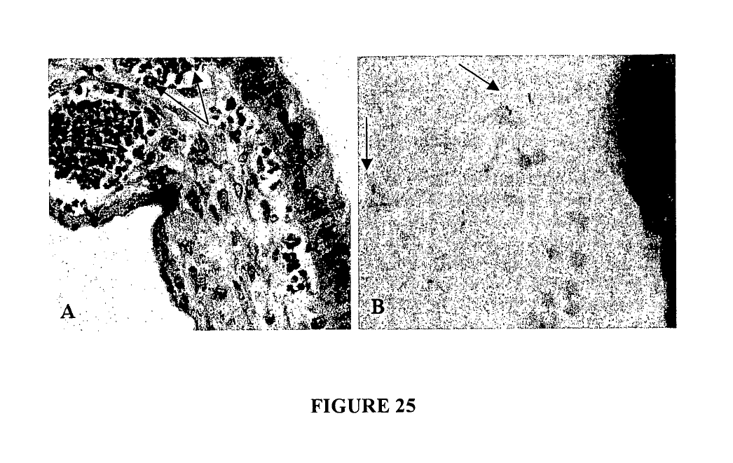

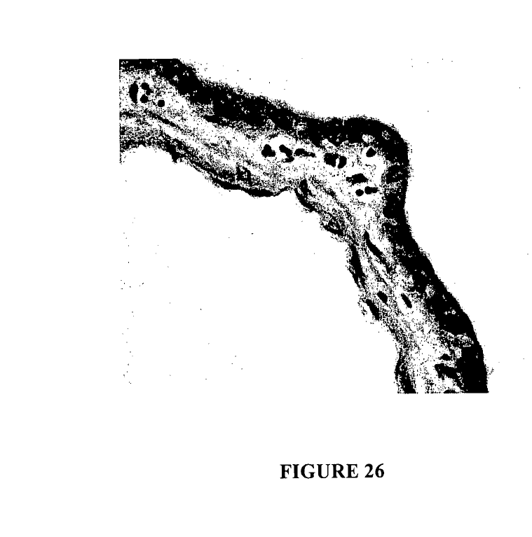

Mice in this group showed markedly slower progression of inflammation in the gall bladder than did mice in Groups 1 and 2, even though Salmonella was first isolated from this organ in mice in Groups 1, 2 and 3 on the same day (Day 5 P.I.). A mild but obvious inflammation was first observed in the gall bladder of one mouse in Group 3 euthanased on Day 5 (FIG. 25). Inflammation sites in which MN cells were predominant were restricted to the lamina propria. However, inflammation was not evident from the gall bladders of mice euthanased on Day 7 and Day 9. Moderate inflammation characterised by enlargement of epithelial cells and mild oedema of the lamina propria was observed on Day 12. Increases in the number of PMNs infiltrating these sites were also seen on Days 12 and 15. Very mild or no apparent inflammation was observed in the gall bladders during the rest of the observation period (FIG. 26). Whilst a small number of putative Salmonella cells were intermittently observed in the gall bladder lumen during the twenty-four day observation period, penetration by the organism of the mucosa or deeper sites of the organ was not evident.

TABLE 27
HISTOPATHOLOGICAL (HP) AND BACTERIAL PENETRATION (BP) SCORES

| | | | Day³ | | | | | | | | | |
|---|---|---|---|---|---|---|---|---|---|---|---|---|
| Score | Group | | 1 | 3 | 5 | 7 | 9 | 12 | 15 | 18 | 21 | 24 |
| HP score¹ | 1 | Liver | 1 | 3 | 3 | ND⁴ | ND | | | | | |
| | | Spleen | 0 | 2 | 3 | ND | ND | | | | | |
| | | Gall bladder | 0 | 1 | 3 | ND | ND | | | | | |
| | 2 | Liver | 0 | 3 | 3 | 2 | ND | | | | | |
| | | Spleen | 0 | 2 | 3 | 2 | ND | | | | | |
| | | Gall bladder | 0 | 0 | 1 | 3 | ND | | | | | |
| | 3 | Liver | 0 | 1 | 2 | 2 | 2 | 1 | 1 | 1 | 1 | 1 |
| | | Spleen | 0 | 0 | 2 | 1 | 1 | 0 | 0 | 0 | 0 | 0 |
| | | Gall bladder | 0 | 0 | 1 | 0 | 0 | 2 | 2 | 0 | 1 | 0 |

| | | | | | | | | | | | |
|---|---|---|---|---|---|---|---|---|---|---|---|
| BP score² | 1 | 0 | 0 | 3 | ND | ND | | | | | |
| | 2 | 0 | 0 | 1 | 3 | ND | | | | | |
| | 3 | 0 | 0 | 1 | 0 | 0 | 1 | 1 | 0 | 1 | 0 |

¹0 = no detectable change, 1 = mild change, 2 = moderate change, 3 = marked change.
²0 = no penetration, 1 = penetration of the lumen only, 2 = penetration of the mucosa, 3 = penetration of the submucosa.
³day post-inoculation,
⁴no data available as no mice survived.

TABLE 28
CHRONOLOGICAL CHANGE IN HISTOPATHOLOGY OF THE LIVER OF MICE
INOCULATED WITH FD436, N-RM4, N-RM25 OR R-NM29 VIA INTRAPERITONEAL ROUTE.

| | Day¹ | | | | | |
|---|---|---|---|---|---|---|
| | Day 1 | | Day 3 | | Day 5 | |
| Group² | Inflammation³ | Score⁴ | Inflammation | Score | Inflammation | Score |
| 1 | D: a very few foci of | 1 | D: randomly distributed | 3 | D: randomly distributed | 3 |
| | inflammatory cells. | | suppurative foci. | | suppurative foci. | |
| | C: PMNs, lymphocytes. | | C: PMNs (predominant). | | C: PMNs, macrophages. | |
| | | | R: focal hepatitis, sinusoidal | | R: hepatitis, coagulative | |
| | | | hypercellularity, increase in | | necrosis, vasculitis of portal | |
| | | | the number of Kupffer cells, | | vessels. | |
| | | | coagulative necrosis. | | | |
| 2 | Nil | 0 | D: randomly distributed | 3 | D: randomly distributed | 3 |
| | | | suppurative foci. | | suppurative foci. | |
| | | | C: PMNs, macrophages. | | C: PMNs. | |
| | | | R: focal hepatitis, coagulative | | R: hepatitis, vasculitis of portal | |
| | | | necrosis, sinusoidal | | vessels. | |
| | | | hypercellularity. | | | |
| 3 | Nil | 0 | D: a few suppurative foci and | 1 | D: randomly scattered | 2 |
| | | | mild periportal MN cell | | suppurative foci but mild | |
| | | | infiltrate. | | periportal MN cell infiltrate. | |
| | | | C: PMNs, lymphocytes, | | C: foci - PMNs (predominant) | |
| | | | ± macrophages. | | Periportal - MN cells. | |
| 5 | Nil | 0 | ND | ND | ND | ND |

| | Day | | | | | |
|---|---|---|---|---|---|---|
| | Day 7 | | Day 9 | | Day 12 | |
| Group | Inflammation | Score | Inflammation | Score | Inflammation | Score |
| 1 | ND | ND | ND | ND | | |
| 2 | D: scattered suppurative foci, | 2 | ND | ND | | |
| | sinusoidal leukocytosis | | | | | |
| | (periportal and portal) | | | | | |
| | C: lymphocytes, plasma cells, | | | | | |
| | macrophages, scattered PMNs. | | | | | |
| | R: prominent extramedullary | | | | | |
| | haematopoiesis. | | | | | |
| 3 | D: scattered suppurative foci and | 2 | D: scattered suppurative foci | 2 | D: a few suppurative foci | 1 |
| | moderate periportal | | and moderate periportal | | and moderate periportal | |
| | inflammatory infiltrate. | | inflammatory infiltrate. | | inflammatory infiltrate | |
| | C: foci - PMNs, lymphocytes | | C: foci - PMNs, lymphocytes | | C: foci - lymphocytes, ± PMNs | |
| | Periportal - MN cells, ± PMNs | | Periportal - lymphocytes, | | periportal→lymphocytes, | |
| | R: prominent extramedullary | | plasma cells, macrophages. | | plasma cells, ± PMNs. | |
| | haematopoiesis and | | R: prominent sinusoidal | | R: sinusoidal | |
| | hepatocellular regeneration. | | leukocytosis, extramedullary | | hypercellularity, | |
| | | | haematopoiesis and | | extramedullary | |
| | | | hepatocellular regeneration.. | | haematopoiesis. | |
| 5 | ND | ND | Nil | 0 | | |

| | Day | | | | | |
|---|---|---|---|---|---|---|
| | Day 15 | | Day 18 | | Day 21 | |
| Group | Inflammation | Score | Inflammation | Score | Inflammation | Score |
| 3 | D: mild periportal | 1 | D: mild periportal | 1 | D: mild periportal | 1 |
| | inflammatory infiltrate and | | inflammatory infiltrate and | | inflammatory infiltrate | |
| | very occasional suppurative | | very occasional, random | | and very occasional, | |
| | foci. | | suppurative foci. | | random suppurative | |
| | C: PMNs, lymphocytes, | | C: PMNs, lymphocytes, | | foci. | |
| | plasma cells. | | plasma cells, macrophages. | | C: lymphocytes, plasma | |
| | R: extramedullary | | R: extramedullary | | cells, macrophages, ± PMNs. | |
| | haematopoiesis and | | haematopoiesis. | | R: sinusoidal | |
| | sinusoidal hypercellularity. | | | | hypercellularity. | |

Day
Day 24

| Group | Inflammation | Score |
|---|---|---|
| 3 | D: mild periportal | 1 |
| | inflammatory infiltrate. | |
| | C: lymphocytes, plasma | |
| | cells, macrophages, ± PMNs. | |

¹Day post-inoculation.
²Mice in each group were intraperitoneally inoculated with the following Salmonella strains; Group 1: FD436; Group 2: N-RM4; Group 3: N-RM25 and Group 4: R-NM29.
³D: distribution, C: cell types, R: remarks, PMN: polymorphonuclear neutrophil granulocyte, MN: mononuclear ±: a small number of, Nil: no histopathological changes were observed, ND: no data available as mice either did not survive or were not euthanased.
⁴Score: histopathological score.

TABLE 29
CHRONOLOGICAL CHANGE IN HISTOPATHOLOGY OF THE SPLEEN OF MICE
INOCULATED WITH FD436, N-RM4, N-RM25 OR R-NM29 VIA INTRAPERITONEAL ROUTE

| | Day 1 | | Day 3 | | Day 5 | |
|---|---|---|---|---|---|---|
| Group² | Inflammation³ | Score⁴ | Inflammation | Score | Inflammation | Score |
| 1 | Nil | 0 | D: scattered suppurative foci | 2 | D: scattered suppurative foci in | 3 |
| | | | in sinus areas. | | sinus areas. | |
| | | | C: macrophages. ± PMNs. | | C: macrophages, scattered and | |
| | | | R: sinusoidal hypercellularity, | | clustered PMNs. | |
| | | | prominent extramedullary | | R: splenitis, necrosis of some | |
| | | | haematopoiesis. | | foci, sinusoidal hypercellularity, | |
| | | | | | prominent extramedullary | |
| | | | | | haematopoiesis. | |
| 2 | Nil | 0 | D: scattered suppurative foci | 2 | D: scattered suppurative foci in | 3 |
| | | | in sinus areas. | | sinus areas. | |
| | | | C: macrophages, scattered and | | C: macrophages, scattered and | |
| | | | clustered PMNs. | | clustered PMNs. | |
| | | | R: sinusoidal hypercellularity. | | R: splenitis, lytic necrosis in | |
| | | | | | sinus areas, vascular thrombosis. | |
| 3 | Nil | 0 | R: very little inflammation - | 0 | D: scattered suppurative foci in | 2 |
| | | | no apparent phagocytic action. | | sinus areas. | |
| | | | | | C: macrophages, ± PMNs. | |
| | | | | | R: sinusoidal hypercellularity, | |
| | | | | | prominent extramedullary | |
| | | | | | haematopoiesis | |
| 5 | Nil | 0 | ND | ND | ND | ND |

| | Day | | | | | |
|---|---|---|---|---|---|---|
| | Day 7 | | Day 9 | | Day 12 | |
| Group | Inflammation | Score | Inflammation | Score | Inflammation | Score |
| 1 | ND | ND | ND | ND | | |
| 2 | D: scattered suppurative | 2 | ND | ND | | |
| | foci in sinus areas. | | | | | |
| | C: macrophages, scattered | | | | | |
| | lymphocytes, plasma cells. | | | | | |
| | R: very few PMNs, | | | | | |
| | prominent extramedullary | | | | | |
| | haematopoiesis. | | | | | |
| 3 | D: occasional random foci | 1 | R: a little number of | 1 | R: moderate lymphoid | 0 |
| | of inflammatory cells in | | lymphocytes in sinusoids, | | hyperplasia. | |
| | sinus areas. | | moderate lymphoid | | | |
| | C: sinus - macrophages. | | hyperplasia. | | | |
| | foci - PMNs. | | | | | |
| | R: prominent | | | | | |
| | extramedullary | | | | | |
| | haematopoiesis. | | | | | |
| 5 | ND | ND | Nil | 0 | | |

| | Day | | | | | |
|---|---|---|---|---|---|---|
| | Day 15 | | Day 18 | | Day 21 | |
| Group | Inflammation | Score | Inflammation | Score | Inflammation | Score |
| 3 | R: mild lymphoid | 0 | R: mild lymphoid hyperplasia. | 0 | R: mild lymphoid hyperplasia. | 0 |
| | hyperplasia | | | | | |

Day
Day 24

| Group | Inflammation | Score |
|---|---|---|
| 3 | R: mild lymphoid | 0 |
| | hyperplasia. | |

¹Day post-inoculation.
²Mice in each group were intraperitoneally inoculated with the following Salmonella strains; Group 1: FD436; Group 2: N-RM4; Group 3: N-RM25 and Group 4: R-NM29.
³D: distribution, C: cell types, R: remarks, PMN: polymorphonuclear neutrophil granulocyte, MN: mononuclear, ±: a small number of, Nil: no histopathological changes were observed, ND: no data available as mice either did not survive or were not euthanased.
⁴Score: histopathological score.

TABLE 30
CHRONOLOGICAL CHANGE IN HISTOPATHOLOGY AND SALMONELLA PENETRATION OF THE
GALL BLADDER OF MICE INOCULATED WITH FD436, N-RM4, N-RM25 OR R-NM29 VIA INTRAPERITONEAL
ROUTE

| | Day¹ | | | | | | | |
|---|---|---|---|---|---|---|---|---|
| | Day 1 | | | | Day 3 | | | |
| Group² | Inflammation³ | Score 1⁴ | Penetration⁵ | Score 2⁴ | Inflammation | Score 1 | Penetration | Score 2 |
| 1 | Nil | 0 | Nil | 0 | R: mild oedema of | 1 | Nil | 0 |
| | | | | | LP. | | | |
| 2 | Nil | 0 | Nil | 0 | Nil | 0 | Nil | 0 |
| 3 | Nil | 0 | Nil | 0 | Nil | 0 | Nil | 0 |
| 5 | Nil | 0 | Nil | 0 | ND | ND | ND | ND |

| | Day | | | | | | | |
|---|---|---|---|---|---|---|---|---|
| | Day 5 | | | | Day 7 | | | |
| Group | Inflammation | Score 1 | Penetration | Score 2 | Inflammation | Score 1 | Penetration | Score 2 |
| 1 | D: LP | 3 | Lumen, | 3 | ND | ND | ND | ND |
| | C: lymphocytes, | | mucosa, | | | | | |
| | monocytes, PMNs. | | submucosa. | | | | | |
| | R: cholecystitis, marked | | | | | | | |
| | epithelial hyperplasia, | | | | | | | |
| | congestion and oedema of | | | | | | | |
| | LP. | | | | | | | |
| 2 | D: LP. | 1 | Lumen. | 1 | D: intraepithelial, | 3 | Lumen, | 3 |
| | C: PMNs, MN cells. | | | | intraluminal, LP. | | mucosa, | |
| | R: mild focal hyperplasia of | | | | C: PMNs (70%), MN | | submucosa. | |
| | epithelial cells. | | | | cells. | | | |
| | | | | | R: cholecystitis, marked | | | |
| | | | | | epithelial hyperplasia, | | | |
| | | | | | mesothelial hyperplasia, | | | |
| | | | | | oedema & haemorrhage | | | |
| | | | | | & fibroplasia in LP, | | | |
| | | | | | subserosal oedema & | | | |
| | | | | | infiltrate. | | | |
| 3 | D: LP. | 1 | Lumen. | 1 | R: very mild epithelial | 0 | Nil | 0 |
| | C: predominantly | | | | hyperplasia. | | | |
| | MN cells, | | | | | | | |
| | ± PMNs. | | | | | | | |
| | R: mild epithelial | | | | | | | |
| | hyperplasia, congestion | | | | | | | |
| | of LP, margination in | | | | | | | |
| | vessels. | | | | | | | |
| 5 | ND | ND | ND | ND | ND | ND | ND | ND |

| Day | |
|---|---|
| Day 9 | Day 12 |

| Group | Inflammation | Score 1 | Penetration | Score 2 | Inflammation | Score 1 | Penetration | Score 2 |
|---|---|---|---|---|---|---|---|---|
| 1 | ND | ND | ND | ND | | | | |
| 2 | ND | ND | ND | ND | | | | |
| 3 | R: very mild | 0 | Nil. | 0 | D: intraepithelial, LP, subserosal. | 2 | Lumen | 1 |
| | oedema of LP. | | | | C: MN cells (70%), PMNs. | | | |
| | | | | | R: moderate epithelial | | | |
| | | | | | hyperplasia, mild oedema of LP, | | | |
| | | | | | subserosal oedema with MN | | | |
| | | | | | inflammatory infiltrate. | | | |
| 5 | Nil | 0 | Nil | 0 | | | | |

| | Day | | | | | | | |
|---|---|---|---|---|---|---|---|---|
| | Day 15 | | | | Day 18 | | | |
| Group | Inflammation | Score 1 | Penetration | Score 2 | Inflammation | Score 1 | Penetration | Score 2 |
| 3 | D: intraepithelial, LP, | 2 | Lumen. | 1 | R: very mild MN | 0 | Nil | 0 |
| | subserosal. | | | | inflammatory infiltrate | | | |
| | C: PMNs | | | | in LP. | | | |
| | (50%), | | | | | | | |
| | MN cells (50%). | | | | | | | |
| | R: moderate epithelial | | | | | | | |
| | hyperplasia, mild | | | | | | | |
| | oedema of LP, | | | | | | | |
| | subserosal | | | | | | | |
| | inflammatory infiltrate. | | | | | | | |

| | Day | | | | | | | |
|---|---|---|---|---|---|---|---|---|
| | Day 21 | | | | Day 24 | | | |
| Group | Inflammation | Score 1 | Penetration | Score 2 | Inflammation | Score 1 | Penetration | Score 2 |
| 3 | D: LP (only a few | 1 | Lumen. | 1 | Nil | 0 | Nil | 0 |
| | suppurative foci). | | | | | | | |
| | C: PMNs (50%), | | | | | | | |
| | MN cells (50%). | | | | | | | |

¹Day post-inoculation.
²Mice in each group were intraperitoneally inoculated with the following Salmonella strains; Group 1: FD436; Group 2: N-RM4; Group 3: N-RM25 and Group 4: R-NM29.
³D: distribution, C: cell types, R: remarks, PMN: polymorphonuclear neutrophil granulocyte, MN: mononuclear, ±: a small number of, LP: lamina propria, Nil: no histopathological changes were observed, ND: no data available as mice either did not survive or were not euthanased.
⁴Score 1: histopathological score, Score 2: bacterial penetration score.
⁵Penetration by inoculated bacteria.

### Example 6

### Vaccine Trials in Mice

### Experimental Procedures for Example 6:

### Experimental Animals:

Mice used in experiments described in this chapter were prepared following the procedure described previously. The number of mice used for each experiment was: 10 for Experiment 1, 35 for Experiment 2 and 18 for Experiment 3. Tables 31, 32 and 33 show the designs adopted to study the three aspects outlined above.

### Vaccine and Challenge Strain Preparation:

N-RM25, N-RM4 and R-NM29 were prepared as vaccines and FD436 for challenge infection according to the procedures described previously. Vaccine doses used in the experiments are shown in Tables 31, 32 and 33. The suspension containing FD436 was prepared to a concentration approximately 100 times greater than its ID₅₀ for mice. Actual challenge doses for the experiments are indicated in Tables 31, 32 and 33.

Experiment 1—Vaccine Trial Using Single Vaccination with a Set Dose of N-RM25

### Study Design:

Ten mice were divided into 2 groups. Mice in Group 1 were intraperitoneally vaccinated once with 100 μl of the vaccine suspension containing N-RM25. Mice in Group 2 were injected with 100 μl of sterile PBS as a control. Challenge infection with FD436 was carried out on Day 21 post-vaccination (P.V.). Vaccine and challenge doses are shown in Table 31.

The clinical appearance of each mouse was monitored daily for 51 days P.V. (30 days post-challenge (P.C.)) in accordance with the method described previously. Faecal pellets from each mouse were collected daily during the same period to examine faecal shedding of both vaccine and challenge strains. Mice which showed severe clinical signs of salmonellosis were euthanased promptly and autopsied. Mice that survived throughout the monitoring period were euthanased on Day 52 P.V. and autopsied. Bacteriological investigation was then conducted on heart blood and gall bladder bile samples collected from euthanased mice to isolate Salmonella. Bacteriological investigations, including enumeration, were also conducted on the liver and spleen of those mice.

Isolation of Salmonella from Faeces, Heart Blood, Gall Bladder Bile and Liver Plus Spleen:

The culture methods for the above samples were as per the methods described previously.

### Statistical Analysis:

Following challenge infection, statistical comparisons were made using Fisher's exact test (two-tailed) comparing the number of surviving mice and the number of mice euthanased due to clinical signs of salmonellosis between the vaccinated and the control groups to evaluate whether vaccination was efficacious.

The number of mice in each group confirmed to be positive or negative for faecal excretion of Salmonella was recorded each day after challenge infection. An analysis using Fisher's exact test (two-tailed) was performed on these data to assess whether the vaccine significantly prevented faecal shedding of the challenge strain.

Experiment 2—Vaccine Trials Using Single and Double Vaccination with Different Doses of N-RM25

### Study Design:

Thirty-five mice were divided into 7 groups. Mice in Groups 1, 3 and 5 were intraperitoneally vaccinated once with 100 μl of graded concentrations of N-RM25. Mice in Groups 2, 4 and 6 were vaccinated twice in the same manner. The booster vaccination (B.V.) was administered on Day 21 P.V. Mice in the control group were injected via the intraperitoneal route with 100 μl of sterile PBS twice at the same interval. Challenge infection was carried out using FD436 intraperitoneally on Day 35 P.V. (Day 14 post-booster vaccination (P.B.V.)). Vaccine and challenge doses are shown in Table 32.

The clinical appearance of each mouse was monitored daily until 65 days P.V. Faecal pellets from each mouse were collected daily during the same period for bacteriological investigation. Mice which showed severe clinical signs of salmonellosis were euthanased promptly and autopsied. Mice that survived throughout the monitoring period were euthanased on Day 66 P.V. and autopsied. Bacteriological investigations were conducted on heart blood and gall bladder bile samples collected from the euthanased mice. Bacteriological investigations, including enumeration, were also performed on the liver and spleen. To further investigate the distribution of Salmonella in vaccinated mice, the following organ samples were also examined: lung, duodenum and jejunum, ileum, ileocaecum, caecum, colon and kidney.

Isolation of Salmonella from Faeces, Heart Blood, Gall Bladder Bile and Liver Plus Spleen:

The culture methods for the above samples were as per the methods described previously.

Isolation of Salmonella from Organ Samples:

Organ samples (lung, duodenum and jejunum, ileum, ileocaecum, caecum, colon and kidney) were placed in boiling water for 3 seconds to sterilise the surface of the organs. Duodenum and jejunum, ileum, ileocaecum, caecum and colon samples were then cut and opened out with a scalpel. Gut contents were gently rinsed from the samples using sterile PBS. Each sample was homogenised and processed for isolation of Salmonella. Isolates identified as Salmonella were further investigated to determine their resistance to nalidixic acid and rifampicin. Bacteriological investigation of the organs was performed only on mice which did not yield Salmonella from a heart blood culture using the DI method due to the difficulty in distinguishing between actual colonisation of the organs by the organism and the temporary presence of the organism in the organs via blood circulation.

### Statistical Analysis:

Following challenge infection, statistical comparisons were made using Fisher's exact test (two-tailed) comparing the number of surviving mice and the number of mice euthanased due to clinical signs of salmonellosis between each of the vaccinated groups and the control. This was done to evaluate the protective properties of N-RM25 administered using different vaccination regimes and doses. When statistically insignificant results were obtained from the above analysis, time periods until death (the time period from challenge infection to euthanasia due to clinical signs of salmonellosis) were also compared using the Student's t test (two-tailed).

The numbers of mice in each group confirmed as positive or negative for faecal excretion of Salmonella were recorded daily throughout the observation period. An analysis using Fisher's exact test (two-tailed) was performed on data obtained before challenge infection. This allowed evaluation of which vaccination methods and doses were the most efficient for minimising post-vaccination faecal shedding. Data obtained post-challenge infection was also analysed using the same test to evaluate which vaccination methods and doses were the most effective in preventing faecal shedding of the challenge strain.

### Experiment 3—Vaccine Trials Using N-RM4 or R-NM29

### Study Design:

Eighteen mice were divided into 3 groups. Mice in Group 1 were vaccinated once via the intraperitoneal route with 100 μl of vaccine solution containing N-RM4. Mice in Group 2 were vaccinated twice with vaccine solution containing R-NM29 in the same manner separated by an interval of 21 days. Mice in Group 3 were intraperitoneally injected twice with 100 μl of sterile PBS at the same interval as a control. Challenge infection was carried out using FD436 via the intraperitoneal route on Day 35 P.V. Vaccine and challenge doses are shown in Table 33.

Clinical appearance of each mouse was monitored daily until 65 days P.V. Faecal pellets from each mouse were collected daily for bacteriological investigation. Mice which showed severe clinical signs of salmonellosis were euthanased promptly and autopsied. Mice that survived throughout the monitoring period were euthanased on Day 66 P.V. and autopsied. Bacteriological investigation was conducted on heart blood and organ samples collected from the euthanased mice. Bacteriological investigations, including enumeration, were also conducted on the liver and spleen.

Isolation of Salmonella from Faeces, Heart Blood, Gall Bladder Bile and Liver Plus Spleen:

The culture procedures for the above samples were the same as those described previously.

Isolation of Salmonella from Organ Samples:

The culture method for the samples followed the procedure described previously.

TABLE 31
S. dublin VACCINE TRIAL IN MICE USING SINGLE
INTRAPERITONEAL VACCINATION WITH A SET DOSE OF
N-RM25.

| | Number of | Vaccine dose¹ | Challenge | Challenge dose |
|---|---|---|---|---|
| Group | mice | (cfu/mouse) | strain² | (cfu/mouse) |
| 1 | 5 | 2.4 × 10⁸ | FD436 | 4.7 × 10⁴ |
| 2 (control) | 5 | 0³ | FD436 | 4.7 × 10⁴ |

¹Vaccination and challenge infection were administered via the intraperitoneal route.
²Challenge infection was carried out on Day 21 P.V.
³Each mouse was injected 100 μl of sterile PBS.

TABLE 32
S. dublin VACCINE TRIALS IN MICE USING SINGLE AND DOUBLE
INTRAPERITONEAL VACCINATION WITH DIFFERENT DOSES
OF N-RM25.

| | | Number of | Vaccination | Vaccination dose | Booster vaccination |
|---|---|---|---|---|---|
| Group | | mice | method | (cfu/mouse) | dose¹ (cfu/mouse) |
| 1 | Low² | 5 | Single | 1.7 × 10³ | N/A |
| 2 | Low | 5 | Double | 1.7 × 10³ | 1.9 × 10⁴ |
| 3 | Medium³ | 5 | Single | 1.7 × 10⁵ | N/A |
| 4 | Medium | 5 | Double | 1.7 × 10⁵ | 1.9 × 10⁶ |
| 5 | High⁴ | 5 | Single | 1.7 × 10⁷ | N/A |
| 6 | High | 5 | Double | 1.7 × 10⁷ | 1.9 × 10⁸ |
| 7 (control)⁵ | N/A | 5 | Double | 0 | 0 |

¹The booster vaccination was carried out on Day 21 P.V. and challenge infection was administered via the intraperitoneal riute using FD436 with a dose of 4.6 × 10⁴ cfu/mouse on Day 35 P.V.
²Low: the low vaccine dose group.
³Medium: the medium vaccine dose group.
⁴High: the high vaccine dose group.
⁵Mice were injected 100 μl of sterile PBS via the intraperitoneal route.

TABLE 33
S. dublin VACCINE TRIALS IN MICE USING
INTRAPERITONEAL VACCINATION WITH
N-RM4 OR R-NM29.

| | | | | | Booster |
|---|---|---|---|---|---|
| | | | | Vaccination | vaccination |
| | Vaccine | Number | Vaccination | dose | dose¹ |
| Group | strain | of mice | method | (cfu/mouse) | (cfu/mouse) |
| 1 | N-RM4 | 5 | Single | 7.5 × 10² | N/A |
| 2 | R- | 8 | Double | 2.7 × 10⁷ | 1.7 × 10⁸ |
| | NM29 | | | | |
| 3 (control) | N/A | 5 | Double | 0² | 0² |

¹The booster vaccination was carried out on Day 21 P.V. and challenge infection was administered via the intraperitoneal route using FD436 with a dose of 7.3 × 10⁴ cfu/mouse on Day 35 P.V.
²Mice were inoculated with 100 μl of sterile PBS via the intraperitoneal route.

### Results for Example 5

Vaccine Trial Using Single Vaccination with a Set Dose of N-RM25

### Observation of Clinical Appearance:

No clinical symptoms were observed in either vaccinated (Group 1) or control (Group 2) mice before challenge infection. The vaccinated mice did not develop any clinical symptoms following challenge infection. In contrast, all mice except one in the control group developed clinical signs of salmonellosis on Day 2 P.C. All mice in this group were euthanased by Day 5 P.C. due to clinical signs of salmonellosis. The mean time until euthanasia among mice in the control group was 115 h. Therefore, it is clear that the protection conferred by the vaccine was significant (p=0.0079).

Isolation of Salmonella from Faeces:

Before challenge infection, the vaccine strain was isolated intermittently from the faeces of vaccinated mice from Day 5 to Day 11 P.V. by the EI method only.

Following challenge infection, Salmonella was isolated from the faeces of the vaccinated mice significantly less often (p<0.0001) than mice in the control group (Table 34). The vaccinated mice excreted Salmonella in the faeces on Day 4 P.C (2 mice), Day 5 (2 mice), Day 7 (3 mice), Day 8 (3 mice), Day 10 (1 mouse) and Day 13 (2 mice) during the 30-day observation period. However, the organism was detectable only by the EI method. These isolates were nalidixic acid and rifampicin sensitive and were identified as the challenge strain (FD436). Compared with this, Salmonella was first isolated by the DI method from the faeces of three control mice on Day 2 P.C. It was then isolated from the faeces of the surviving mice continuously until all mice in this group were euthanased (Day 5 P.C.).

### Autopsy:

Although there was slight to moderate splenomegaly in two mice in the vaccinated group, no other pathological changes were observed in this group. In contrast, accumulation of cloudy, straw-colour fibrinous exudate, identified as pyogenic exudate, was observed in the abdominal cavity in the control mice. All mice in this group developed splenomegaly and some of them also developed petechial haemorrhages of the serosa of the visceral organs, especially the liver, and the peritoneum. There were small grey-white foci scattered across the entire surface of the livers of two control mice.

Isolation of Salmonella from Heart Blood, Gall Bladder Bile and Liver Plus Spleen:

Salmonella was not isolated from heart blood, gall bladder bile or the liver plus spleen specimens of any of the vaccinated mice. On the other hand, the challenge strain was isolated from all of these specimens from the control mice using the DI method. The mean number of organisms in the liver and spleen of the control mice was 5.0×10⁸ cfu/g (the number ranging between 2.0×10⁸ and 1.0×10⁹ cfu/g)—see Table 34.

TABLE 34
S. dublin VACCINE TRIAL IN MICE USING SINGLE
INTRAPERITONEAL VACCINATION WITH A SET DOSE OF
N-RM25
FOLLOWED BY CHALLENGE INFECTION WITH WILD STRAIN
FD436.

Group¹

| | |
|---|---|
| 1 | 2 (control) |

| | | | |
|---|---|---|---|
| Number of | Initial number | 5 | 5 |
| surviving | of mice | | |
| Mice² | Pre-challenge | 5 | 5 |
| | Post-challenge | 5 | 0⁷ |

| | | |
|---|---|---|
| Mean time until death³ (h) | N/A | 115 |
| Protection (%) | 100 | 0 |
| Faecal Salmonella isolation⁴ | 16/100 | 0/100 |
| (pre-challenge) | | |
| Faecal Salmonella isolation⁴ | 13/150 | 12/19 |
| (post-challenge) | | |
| Heart blood culturing⁵ | 0/5 | 5/5 |
| Gall bladder bile culturing⁵ | 0/5 | 5/5 |
| Isolation of Salmonella from | 0/5 | 5/5 |
| liver & spleen | | |
| Mean number of Salmonella in | 0 | 5.0 × 10⁸ |
| the liver & spleen⁶ (cfu/g) | | |

¹Vaccine dose for Group 1: 2.4 × 10⁸ cfu/mouse. Group 2 mice received 100 μl of sterile PBS. Challenge dose: 4.7 × 10⁴ cfu/mouse (on Day 21 P.V.)
²Number of surviving mice in the group on Day 20 P.V. (pre-challenge) and Day 51 P.V. (post-challenge).
³For ethical reasons, the time from challenge infection to euthanasia due to critical signs of salmonellosis was used to derive this value.
⁴Number of cultures positive for the vaccine strain (pre-challenge) and the challenge strain (post-challenge) over the total number of samples tested in each group.
⁵Number of cultures positive for the challenge strain over the number of samples tested.
⁶Mean number of the challenge strain organisms isolated from the liver plus spleen samples in each group.
⁷All mice were euthanased by Day 5 P.C. due to critical signs of salmonellosis.

Vaccine Trials Using Single and Double Vaccination with Different Doses of N-RM25

### Observation of Clinical Appearance:

Prior to challenge infection, no mice showed any clinical symptoms. Following lethal challenge infection, no mice vaccinated with high doses (Groups 5 and 6) showed clinical signs of salmonellosis irrespective of whether the single or the double dose was given. In comparison with this, only one mouse of each group given medium-dose vaccination (Groups 3 and 4) survived throughout the observation period. All mice in the low-dose vaccination groups (Groups 1 and 2) and the control group developed clinical signs of salmonellosis and were euthanased. Based on the above results, N-RM25 vaccination with high-doses conferred significantly greater protection against challenge infection than vaccination with low and medium doses and the control (high-dose groups vs. medium-dose groups: p=0.0007, vs. low-dose groups and control: p<0.0001). There were no significant differences in protection between the groups receiving low or medium levels of vaccination and the control group (Table 35).

Isolation of Salmonella from Faeces:

No mice given the low-dose vaccines shed Salmonella in the faeces during the pre-challenge period. In contrast, the vaccine strain was isolated from the faeces of some mice in medium and high dose groups during the period. Statistical analysis showed that mice in the double medium-dose group and the high-dose groups excreted the vaccine strain in the faeces significantly more often than mice in both the low-dose group and control group during the period between the first vaccination and challenge infection (low-dose group or control group vs. double medium dose group: p=0.0147, vs. high dose groups: p<0.0001). However, isolation was intermittent and only for short periods (equal to or less than 7 days) following each vaccination and was only possible with EI method. There was no significant difference in the frequency of faecal excretion of the organism between the single vaccination groups and the double vaccination groups.

Following challenge infection, the challenge strain was isolated from the faeces of only two mice in the double high-dose vaccination group (Group 6) (once on Day 9 P.C. from the faeces of one of the two mice, and from the faeces of the other mouse intermittently until 22 days P.C. and then not isolated again during the observation period). These isolates were only obtained using the EI method. The organism was isolated intermittently throughout the observation period from the faeces of three out of five mice in the single high-dose vaccination group (Group 5). Compared with the high-dose groups, the challenge strain was continuously isolated from the faeces of the mice in the control group using the DI method from Day 2 P.C. to the day on which all mice were euthanased. All mice in the single low-dose group (Group 1) and some in the double low dose group (Group 2) started excreting the strain on Day 3 P.C. and Day 4 P.C., respectively. The strain was then isolated from the faeces of all surviving mice in those groups continuously until the day on which the last mouse in each group was euthanased. Similarly, mice in both the medium vaccination groups first excreted the organism on Day 4 P.C. and surviving mice then did so throughout the observation period. Based on this, following challenge infection, the challenge strain was isolated from faeces of mice in the high-dose groups significantly less often than the faeces of the medium-dose, low-dose and control groups (high-dose groups vs. medium-dose and low-dose groups: p<0.0001, vs. control group: p=0.002). In the high-dose group, furthermore, mice vaccinated with the double dose excreted the strain significantly p<0.0001) less often than did mice vaccinated with the single dose. There were no significant differences in excretion of the strain between the low-dose and the medium-dose vaccination groups and the control group (Table 35).

### Autopsy:

No gross lesions were observed upon autopsy of the mice in the double high-dose group. Three of five mice given the single high-dose vaccine developed only mild pathological changes, such as mild splenomegaly. Compared with the high-dose groups, the surviving mice in the medium-dose groups developed somewhat severer changes, including splenomegaly and a few small, grey-white foci on the surface of the liver and the kidney. On the other hand, twenty-two out of twenty-three mice euthanased due to clinical signs of salmonellosis in the medium-dose and low-dose groups developed from moderate to marked pathological changes. The changes include splenomegaly, petechial haemorrhages on visceral organs, grey-white foci on the surface of the liver and cloudy straw-colour fibrinous exudate in the abdominal cavity. Some euthanased mice also developed grey-white foci on the surface of the spleen and kidney and grey-white fibrinous exudate in the thoracic cavity.

Isolation of Salmonella from Heart Blood and Gall Bladder Bile:

Salmonella was not isolated even using EI method from heart blood and gall bladder bile of mice in the high-dose groups. In contrast, the challenge strain was isolated from the heart blood of eight out of ten mice in medium-dose groups, all mice in the low-dose and the control groups. All of these mice were euthanased due to clinical signs of salmonellosis. Although gall bladder bile specimens could not be obtained from all of the mice, of the specimens that were taken, the challenge strain was isolated only from those collected from mice euthanased due to clinical sings of salmonellosis (Table 35).

Quantitative and Qualitative Isolation of Salmonella from Liver Plus Spleen:

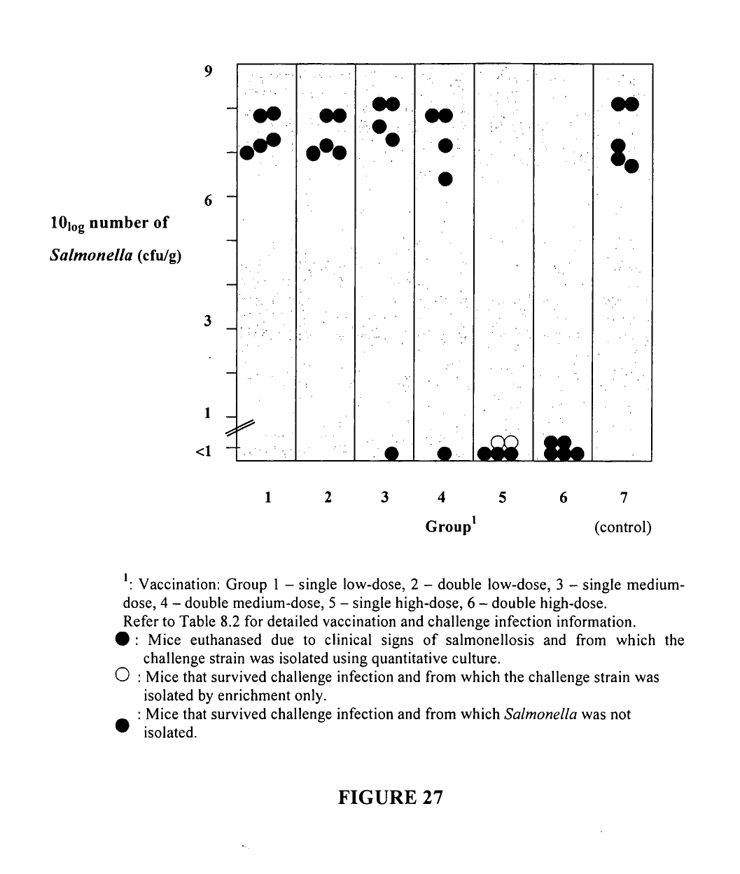

No Salmonella was isolated from liver plus spleen specimens collected from mice given double high-dose vaccination using either quantitative or qualitative methods. The challenge strain was isolated from two mice in the single high-dose group, but only with the qualitative method. In contrast, the strain was isolated quantitatively from mice in the medium-dose and low-dose groups and the control group that were euthanased due to clinical signs of salmonellosis. The numbers of the organism in the organs ranged between 4.9×10⁷ and 1.5×10⁸, and 3.6×10⁶ and 9.3×10⁷ cfu/g for single and double medium-dose vaccination groups, respectively, from 1.4×10⁷ to 8.8×10⁷ for the low-dose groups and between 7.5×10⁶ and 1.6×10⁸ for the control group (Table 35 and FIG. 27). The challenge strain was not isolated from the liver and spleen of mice given medium-dose vaccination that survived challenge. The vaccine strains were not isolated from any liver plus spleen specimen.

Isolation of Salmonella from Organ Samples:

As shown in Table 36, in the double high-dose group, the challenge strain was isolated only from the ileocaecum of one out of five mice using the EI method. Compared with this, the strain was isolated from many organs of mice in the other groups. The strain was isolated from the liver, spleen, ileum, ileocaecum, caecum, colon and kidney of three of five mice given single high-dose vaccination. The strain was also isolated from the intestine between the ileum and the colon of mice in the medium-dose groups. The vaccine strain was isolated from the ileocaecum and colon of one mouse in the double high-dose group only by using the EI method.

TABLE 35
S. dublin VACCINE TRIALS USING SINGLE AND DOUBLE VACCINATION WITH DIFFERENT DOSES
OF N-RM25 FOLLOWED BY CHALLENGE INFECTION WITH WILD STRAIN FD436.

| | | Group¹ | | | | | | |
|---|---|---|---|---|---|---|---|---|
| | | | | | | | | 7 |
| | | 1 | 2 | 3 | 4 | 5 | 6 | (control) |
| Number of | Initial number | 5 | 5 | 5 | 5 | 5 | 5 | 5 |
| surviving mice | of mice | | | | | | | |
| | Pre-challenge | 5 | 5 | 5 | 5 | 5 | 5 | 5 |
| | Post-challenge | 0 | 0 | 1 | 1 | 5* | 5* | 0 |
| Mean time until death² (h) | | 115 | 216 | N/A | N/A | N/A | N/A | 100 |
| Protection (%) | | 0 | 0 | 20 | 20 | 100 | 100 | 0 |
| Faecal | Pre-challenge | 0/170 | 0/170 | 3/170 (3)⁵ | 7/170** (7) | 14/170** (14) | 18/170** (18) | 0/170 |
| Salmonella | Post-challenge | 9/24 (0) | 23/45 (1) | 34/58 (13) | 37/66 (20) | 52/155 (45) | 12/155*** (12) | 11/21 (1) |
| isolation³ | | | | | | | | |

| | | | | | | | |
|---|---|---|---|---|---|---|---|
| Heart blood culture⁴ | 5/5 | 5/5 | 4/5⁶ | 4/5⁶ | 0/5 | 0/5 | 5/5 |
| Gall bladder bile culture⁴ | 1/1 | 2/2 | 2/3⁶ | 1/2⁶ | 0/5 | 0/5 | 4/4 |
| Isolation of Salmonella from liver | 5/5 | 5/5 | 4/5⁶ | 4/5⁵ | 2/5 (2) | 0/5 | 5/5 |
| plus spleen⁴ | | | | | | | |

¹Vaccination: Group 1 = single low-dose, 2 = double low-dose, 3 = single medium-dose, 4 = double medium-dose, 5 = single high-dose, 6 = double high-dose. Booster vaccination: on Day 21 P.V., challenge infection: on Day 35 P.V.. Refer to Table 8.2 for detailed vaccination and challenge infection information.
²For ethical reasons, the time period from challenge infection to euthanasia due to critical illness was used in the calculation of the mean time until death.
³Numbers of cultures positive for the corresponding vaccine strains (pre-challenge) and the challenge strain (post-challenge) over the total number of samples tested in each group.
⁴Number of cultures positive for the challenge strain over the number of samples tested at autopsy.
⁵Numbers in parentheses indicate the number of positive results detected only using the EI method.
⁶All mice that produced negative results survived throughout the observation period.
*Control vs. p = 0.0079;
**control vs. Group 4 p = 0.0147, Group 5 p < 0.0001, Group 6 p < 0.0001;
***control vs. p < 0.0001 (two-tailed Fisher's exact test).

TABLE 36
Distribution of Salmonella in internal organs of mice that survived
throughout vaccine trials using single and double vaccination methods
with different doses of N-RM25 followed by challenge infection with wild
strain FD436.

| | Group¹ | | | |
|---|---|---|---|---|
| | 3 | 4 | 5 | 6 |
| Organ | (n = 1) | (n = 1) | (n = 5) | (n = 5) |
| Lung | 0/1² | 0/1 | 0/5 | 0/5 |
| Liver & spleen | 0/1 | 0/1 | 2S/5 (2) | 0/5 |
| Duodenum & | 0/1 | 0/1 | 0/5 | 0/5 |
| jejunum | | | | |
| Ileum | 1S/1 (1)³ | 1S/1 (1) | 1S/5 (1) | 0/5 |
| Ileocaecum | 1S/1 (1) | 1S/1 | 3S/5 (2) | 1S, 1R/5 (2) |
| Caecum | 0/1 | 1S/1 (1) | 1S/5 (1) | 0/5 |
| Colon | 1S/1 (1) | 1S/1 (1) | 3S/5 (2) | 1R/5 (1) |
| Kidney | 0/1 | 0/1 | 1S/5 | 0/5 |

¹Vaccination: Group 3 = single medium-dose, 4 = double medium-dose, 5 = single high-dose, 6 = double high-dose. Refer to Table 32 for detailed vaccination and challenge infection information. Organ sample culturing was not performed on mice in Group 1 (single low-dose) and Group 2 (double low-dose) and the control because their heart blood samples were positive for Salmonella.
²Results represent the numbers of cultures positive for the vaccine strain and the challenge strain over the total number of samples tested in each group. S: nalidixic acid and rifampicin sensitive (challenge) strain, R: nalidixic acid and rifampicin resistant (vaccine) strain.
³Numbers in parentheses indicate the number of isolates obtained using EI method.

### Vaccine Trials Using N-RM4 or NM29:

All mice vaccinated with N-RM4 showed clinical symptoms following vaccination and one of them died due to acute septicaemia caused by the vaccine strain before challenge infection (Day 8 P.V.). The remaining mice were moderately ill (the mice had shown clinical signs in two or fewer of the five parameters) until Day 12 P.V. then recovered. No other clinical signs were observed in the remaining mice before challenge infection. No mice in this group that survived after vaccination showed clinical symptoms following challenge infection.

Mice receiving R-NM29 and mice in the control group developed no clinical signs before challenge infection (Table 37). Four out of eight mice in the R-NM29 group were euthanased due to clinical signs of salmonellosis on Day 7 P.C. (1 mouse), Day 8 P.C. (2 mice) and Day 12 P.C. (1 mouse). Whilst some of the remaining mice in this group were moderately ill between Days 5 and 13 P.C., no more mice were euthanased. All mice in the control were euthanased due to clinical signs of salmonellosis by 4 days P.C. (Table 37).

Isolation of Salmonella from Faeces:

Two mice vaccinated with N-RM4 first excreted this vaccine strain on Day 3 P.V. The strain was then isolated intermittently from mice in this group not only before but also after challenge infection. Mixed isolation of the vaccine strain and the challenge strain was often observed from faecal samples in this group (Table 37).

Salmonella was not isolated from faecal samples collected from the mice in the R-NM29 group before challenge infection. A mouse in this group first excreted the challenge strain on Day 3 P.C. This organism was then isolated from all mice in this group almost everyday until the end of the observation period.

The organism was isolated from one mouse in the control group on the day following challenge infection. All mice in this group then excreted the organism continuously until the mice were euthanased (Table 37).

### Autopsy:

The N-RM4 vaccinated mouse that died on Day 8 P.V. developed marked pathological changes, such as cloudy straw-colour fibrinous exudate in the thoracic and abdominal cavities, petechial haemorrhages of serosa of visceral organs, splenomegaly and grey-white foci scattered on the surface of the liver and the spleen. Three mice in the same group which survived throughout the observation period showed only mild to moderate pathological changes, such as splenomegaly. The other survivor had a few small grey-white foci on the surface of the kidney.

Four mice in the R-NM29 group euthanased due to clinical signs of salmonellosis developed splenomegaly and petechial haemorrhages of serosa of visceral organs. Two of these mice had marked fibrinous exudate in the abdominal cavity and small grey-white foci scattered across the entire surface of the liver. The surviving mice in this group showed few small grey-white foci on the surface of the liver.

Isolation of Salmonella from Heart Blood:

As indicated in Table 37, Salmonella was not isolated even using the EI method from heart blood of mice which survived throughout the observation period. In contrast, the challenge strain was isolated from all heart blood specimens from mice in R-NM29 group and the control group which were euthanased due to clinical signs of salmonellosis.

Quantitative and Qualitative Isolation of Salmonella from Liver Plus Spleen:

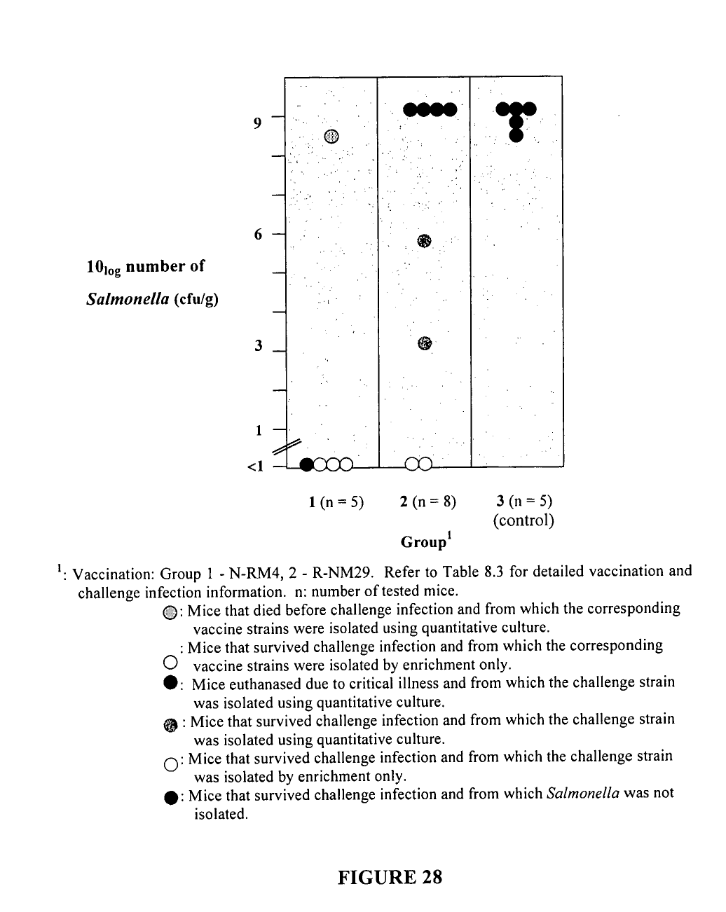

A high number of Salmonella (6.5×10⁸ cfu/g) was isolated from a liver plus spleen specimen of the mouse which died 8 days after vaccination with N-RM4. Salmonella was isolated only with the qualitative method from the organs of three of the four remaining mice in this group that survived throughout the observation period. Interestingly, only the vaccine strain was isolated from two of the three mice and the challenge strain was the only isolate from the remaining mouse (Tables 37 and 38 and FIG. 28).

When R-NM29 was used as a vaccine, the challenge strain was isolated in numbers that could be quantified from the liver plus spleen specimens of four mice that were euthanased due to salmonellosis. The challenge strain was also isolated in numbers that could be quantified from two out of four mice in this group which survived throughout the observation period. However, the strain was only isolated following enrichment from the other two mice. Based on the results of quantitative measurements, the number of the organisms in the organs of the euthanased mice ranged between 1.1×10⁹ and 2.4×10⁹ cfu/g (with a mean of 1.8×10⁹), as compared with between 0 and 7.2×10⁵ cfu/g (with a mean of 1.8×10⁵) in the mice that survived challenge infection (Tables 37 and 38 and FIG. 28).

The challenge strain was isolated quantitatively from the organs of control mice. The numbers ranged between 5.3×10⁸ and 3.3×10⁹ cfu/g (with a mean of 1.7×10⁹)—see Table 37 and FIG. 28.

Isolation of Salmonella from Organ Samples:

Organ sample culturing was conducted for samples collected from 8 mice (4 from each vaccinated group) which survived throughout the observation period, as negative results for heart blood culturing were obtained from these mice. In the N-RM4 group, only the challenge strain was isolated from one out of four mice and the vaccine strain was the only isolate from the remaining mice. The organs from which Salmonella was isolated were the liver and spleen, ileocaecum and kidney (Table 38).

The challenge strain was the only isolate from organ samples of R-NM29 vaccinated mice. The organs from which the organism was isolated were the liver and spleen, ileocaecum, caecum, colon and kidney (Table 38).

TABLE 37
S. dublin VACCINE TRIALS IN MICE USING N-RM4 OR R-NM29
FOLLOWED BY CHALLENGE INFECTION WITH WILD STRAIN
FD436.

| | | Group | | |
|---|---|---|---|---|
| | | 1⁴ | 2 | 3 (control) |
| Number of | Initial number of mice | 5 | 8 | 5 |
| surviving mice | Pre-challenge | 4 | 8 | 5 |
| | Post-challenge | 4 | 4 | 0 |
| Protection (%) | | 100 | 50 | 0 |
| Faecal | Pre-challenge | 80/143 | 0/272 | 0/170 |
| Salmonella | Post-challenge | 22/72 | 65/106 | 11/20 |
| isolation² | | (23/72)⁵ | | |

| | | | |
|---|---|---|---|
| Heart blood culturing³ | 0/4 | 4/8 | 5/5 |
| Isolation of Salmonella from liver plus | 1/4 (2/4)⁵ | 8/8 | 5/5 |
| spleen³ | | | |

¹Vaccination: Group 1 = N-RM4, 2 = R-NM29. Booster vaccination: on Day 21 P.V. (only for Group 2). Challenge infection: on Day 35 P.V. Refer to Table 33 for detailed vaccination and challenge infection information.
²Number of cultures positive for the vaccine strain (pre-challenge) and the challenge strain (post-challenge) over the total number of samples tested in each group.
³Number of cultures positive for the challenge strain over the number of samples tested at autopsy.
⁴Results of protection, faecal Salmonella isolation, heart blood culturing and isolation of Salmonella from liver plus spleen do not include data obtained from the mouse which died before challenge infection.
⁵Numbers in parentheses indicate the number of cultures positive for the vaccine strain over the total number of samples.

TABLE 38
DISTRIBUTION OF Salmonella IN INTESTINAL ORGANS OF MICE
THAT SURVIVED THROUGHOUT VACCINE TRIALS USING
N-RM4 OR R-NM29 FOLLOWED BY CHALLENGE INFECTION
WITH FD436.

Group¹

| | 1 | 2 |
|---|---|---|
| Organ | (n = 4) | (n = 4) |
| Lung | 0/4² | 0/4 |
| Liver & spleen | 1S, 2R/4 | 4S/4 |
| Duodenum & jejunum | 0/4 | 0/4 |
| Ileum | 0/4 | 0/4 |
| Ileocaecum | 1S, 1R/4 | 3S/4 |
| Caecum | 0/4 | 3S/4 |
| Colon | 0/4 | 2S/4 |
| Kidney | 1S, 2R/4 | 1S/4 |

¹Vaccination: Group 1 = N-RM4, 2 = R-NM29. Refer to Table 33 for detailed vaccination and challenge infection information.
²Results represent the numbers of cultures positive for the corresponding vaccine strains and the challenge strain over the total number of samples tested in each group. S = nalidixic acid and rifampicin sensitive (challenge) strain, R = nalidixic acid and rifampicin resistant (vaccine) strains.

### Example 7

### Vaccine Trials in Calves

### Experimental Procedures for Example 7

### Experimental Animals:

For the first vaccine trial, eight 3 to 4 week old male calves (Calf No. 1-8) will be obtained from a herd known to be free of S. dublin infection and divided into 4 groups. The calves will be given colostrum immediately after birth. Prior to the trials, faeces and sera from each calf will be tested bacteriologically and serologically to confirm the calves are free from S. dublin infection. The same process will be followed for a further 8 male calves (Calf No. A-H) for the second vaccine trial.

### Feeding:

Milk replacement formula will be given twice a day (8 a.m. and 3 p.m.).

### Clinical Monitoring:

Clinical condition of the calves will be monitored three times a day at 9 a.m., 4 p.m. and 8 p.m.

### Vaccination and Challenge Infection:

In the first trial, calves in groups 2 and 3 will be orally vaccinated and then calves in groups 3 and 4 will be challenge exposed via the oral route 14 days after vaccination (see Tables 1 and 2). In the second trial, calves in groups 6 and 7 will be vaccinated and then calves in groups 7 and 8 will be challenge exposed in the same manner (see Tables 39 and 41).

TABLE 39
VACCINATION AND CHALLENGE INFECTION FOR
FIRST TRIAL

| | | Number | | Dose | | Challenge |
|---|---|---|---|---|---|---|
| | | of | Vaccine | (cfu) & | Challenge | dose (cfu) & |
| | Group | animals | strain | route | strain | route |
| 1^{st} | 1 | 2 | N/A | — | N/A | — |
| trial | 2 | 2 | M25 | 10^{11#} PO | N/A | — |
| | 3 | 2 | M25 | 10^{11#} PO | FD436 | 10^{9#} PO |
| | 4 | 2 | N/A | — | FD436 | 10^{9#} PO |
| 2^{nd} | 5 | 2 | N/A | — | N/A | — |
| trial | 6 | 2 | M25 | 10^{11#} PO | N/A | — |
| | 7 | 2 | M 5 | 10^{11#} PO | FD436 | 10^{9#} PO |
| | 8 | 2 | N/A | — | FD436 | 10^{9#} PO |

^{#}To ensure the viability of bacteria after passage through the stomach, the following chemical solution will be administered with the bacteria: 1 g MgCO₃, 1 g magnesium trisilicate, 1 g NaHCO₃, 20 ml distilled water.

### Study Design:

1. Clinical signs will be monitored for 8 weeks following vaccination.

2. Faeces, whole blood and serum will be taken for bacteriological, haematological, biochemical and serological examination (Tables 2 and 3).

3. The calves will be euthanised on Day 56 or upon showing clinical signs of salmonellosis and subject to full post-mortem examination including obtaining tissue specimens for bacteriological and histological examination.

Organs and contents (cont.) that will be bacteriologically investigated are as follows: Lung, liver, spleen, kidney, abomasal cont., duodenal cont., jejunal cont., ileal cont., ileocaecal cont., caecal cont., colonic cont., ileal mesenteric lymph nodes (MLN), ileocaecal MLN, colonic MLN and bile.

Organs that will be histopathologically investigated are as follows: Liver, spleen, gall bladder, kidney, duodenum, jejunum, ileum, ileocaecum, caecum, colon and mesenteric lymph nodes. Gall bladders will be further investigated through electron microscopy.

The vaccine trial will be repeated three times to obtain adequate numbers for statistical confidence.

### Results for Example 7

1. No calves produced clinical symptoms, except soft stools, post-vaccination using M25.

2. Salmonella was isolated from faeces of vaccinated calves using the direct isolation method for 1-2 days post-vaccination. The organism was not isolated from faeces beyond 8 days following vaccination even using the enrichment method.

3. Post lethal challenge infection, all vaccinated calves had a fever (39.5-40.5° C.) and watery diarrhoea for several days, but they were relatively active and had a stable appetite. Compared with this, calves in the positive control group (non-vaccinated group) produced critical symptoms, such as very high fever (>40.5° C.), depression, anorexia, tachypnea and tachycardia, but only a small amount of diarrhoea.

4. Numbers of Salmonella (a nalidixic acid and rifampicin sensitive strain that was suspected of being the challenge strain) excreted in the faeces of vaccinated calves following challenge infection were significantly (p=0.0242) fewer than those in the faeces of calves in the positive control group.

5. Upon autopsy, Salmonella (the challenge strain) was isolated from various organs and tissues, including the lung, kidney, bile, bone marrow and cerebrospinal fluid, of calves in the positive control group. However, in the vaccinated and challenge group, the strain was isolated only from the intestine and mesenteric lymph node.

6. In the vaccinated and challenge group, no Salmonella was isolated from any organs tested 32 days post-vaccination (18 days post-challenge).

### Current Findings:

1. Single vaccination with M25 protected calves from lethal challenge infection.

2. The vaccine strain was excreted in the faeces only for a short period (<8 days).

3. The vaccine eliminated the challenge bacteria from the host within 18 days.

TABLE 40
Salmonella ISOLATION FROM FAECES AND BLOOD COLLECTED FROM CALVES IN VACCINE TRIALS

| | | Day | | | | | | | | | | | | | | | | | | | | |
|---|---|---|---|---|---|---|---|---|---|---|---|---|---|---|---|---|---|---|---|---|---|---|
| Calf | Sample | 0 | 1 | 2 | 3 | 4 | 5 | 8 | 11 | 14 | 15 | 16 | 17 | 18 | 19 | 22 | 25 | 28 | 31 | 34 | 37 | 40 |
| 1 | Faeces | − | − | − | − | − | − | − | − | − | − | − | Euthanased | | | | | | | | | |
| (NC) | Blood | − | − | − | | | − | − | − | − | − | − | | | | | | | | | | |
| D | F | − | +DR | +ER | +ER | +ER | +ER | − | Euthanased | | | | | | | | | | | | | |
| (V) | B | − | − | − | | | + | − | | | | | | | | | | | | | | |
| 3 | F | − | +DR | +ER | +ER | +ER | +ER | +ER | − | − | − | − | − | − | − | − | − | − | − | − | − | − |
| (V) | B | − | + | − | | | − | − | − | − | − | − | − | − | − | − | − | − | − | | − | − |
| 4 | F | − | +DR | +ER | − | − | − | − | − | − | − | − | − | − | − | − | − | − | − | − | − | − |
| (V) | B | − | − | − | | | − | − | − | − | − | − | − | − | − | − | − | − | − | | − | − |

| 7 | F | − | − | − | +E* | +E* | − | − | − | +E* | 3 × 10⁸ | 3 × 10⁸ | Dead | | | | | | | |
|---|---|---|---|---|---|---|---|---|---|---|---|---|---|---|---|---|---|---|---|---|
| (PC) | B | − | − | − | | | − | − | − | − | + | + | | | | | | | | |
| 8 | F | − | − | − | − | − | − | − | − | − | 2 × 10⁷ | 5 × 10⁸ | Euthanased | | | | | | | |
| (PC) | B | − | − | − | | | − | − | − | − | + | + | | | | | | | | |
| G | F | − | − | − | − | − | − | − | − | − | 9 × 10⁷ | 2 × 10⁶ | 2 × 10⁶ | Euthanased | | | | | | |
| (PC) | B | − | − | − | | | − | − | − | − | + | + | + | | | | | | | |
| H | F | − | − | − | − | − | − | − | − | − | 7 × 10⁷ | 7 × 10⁷ | 8 × 10⁷ | Euthanased | | | | | | |
| (PC) | B | − | − | − | | | − | − | − | − | − | − | + | | | | | | | |
| E | F | − | +DR | +ER | − | +ER | +ER | +ER | − | − | 4 × 10⁶ | 2 × 10⁶ | +E | Euthanased | | | | | | |
| (VC) | B | − | − | − | | | + | − | − | − | − | − | − | | | | | | | |
| F | F | − | +DR | +ER | − | +ER | +ER | − | − | − | 1 × 10⁷ | 1 × 10⁶ | +E | Euthanased | | | | | | |
| (VC) | B | − | − | − | | | − | + | − | − | − | − | − | | | | | | | |
| 5 | F | − | +DR | +ER | − | +ER | − | − | − | − | 3 × 10⁶ | +E | +E | +E | − | − | − | − | − | Euthanased |
| (VC) | B | − | − | − | | | − | − | − | − | − | − | − | − | − | − | − | − | − | |
| 6 | F | − | +DR | +DR | +ER | +ER | − | − | − | − | 2 × 10⁶ | 6 × 10⁵ | 3 × 10⁵ | +E | +E | +E | − | − | − | Euthanased |
| (VC) | B | − | − | − | | | − | − | − | − | − | − | − | − | − | − | − | − | − | |
| C | F | − | +DR | +ER | +ER | +ER | − | − | − | − | 4 × 10⁶ | 1 × 10⁵ | +E | +E | − | − | − | − | − | Euthanased |
| (VC) | B | − | − | − | | | − | − | − | − | − | − | − | − | − | − | − | − | − | |
| 2 | F | − | − | − | − | − | − | − | − | − | − | − | − | − | − | − | +E | − | | Euthanased |
| (NC) | B | − | − | − | | | − | − | − | − | − | − | − | − | + | + | + | + | | |

| | | | | | | | | | | | | | |
|---|---|---|---|---|---|---|---|---|---|---|---|---|---|
| A | F | − | − | − | − | − | − | − | +E | − | − | 4 × 10⁸ | Euthanased |
| (NC) | B | − | − | − | | | − | − | + | + | + | + | |
| B | F | − | − | − | − | +E | − | +E | +E | +E | − | − | Euthanased |
| (NC) | B | − | − | − | | | − | − | + | + | − | + | |

Day 0 = Vaccination;
Day 14 = Challenge infection;
NC = Negative control;
PC = Positive control;
V = Vaccinated;
VC = Vaccinated and challenged;
+ = positive;
− = negative;
D = direct isolation method;
E = enrichment isolation method;
R = Nalidixic acid & rifampicin resistant strain;
*= Salmonella other than S. dublin.

TABLE 41
Salmonella ISOLATION FROM ORGAN, TISSUE AND CONTENT SAMPLES COLLECTED FROM
CALVES IN VACCINE TRIALS

| | | Autopsy | Organ, tissue and content | | | | | | | | | | | |
|---|---|---|---|---|---|---|---|---|---|---|---|---|---|---|
| Group | Calf | Date | D.C | I.C. | C.C. | M.L.N | Liver | Spleen | Bile | Kidney | Lung | A.S | B.M | C.S.F |
| Negative | 1 | Day 16 | − | − | − | − | − | − | − | − | − | − | − | − |
| control | A | 16 | 5 × 10⁶ | 5 × 10⁶ | 7 × 10⁶ | + | 2 × 10⁶ | 2 × 10⁶ | 3 × 10⁷ | + | + | − | − | − |
| | B | 16 | +E | +E | E+ | + | 1 × 10⁵ | 4 × 10⁴ | − | + | + | − | − | − |
| | 2 | 30 | − | +E | − | − | − | − | − | − | − | − | − | − |
| Positive | 8 | 16 | 7 × 10⁵ | 3 × 10⁷ | 1 × 10⁷ | + | 1 × 10⁴ | 2 × 10⁵ | − | + | + | − | + | + |
| control | 7 | 17 | | | | + | + | + | + | + | + | + | + | + |
| (Challenge | | (Dead) | | | | | | | | | | | | |
| only) | G | 18 | 2 × 10⁴ | 5 × 10⁴ | 2 × 10⁵ | + | 2 × 10³ | 2 × 10³ | − | + | − | − | − | − |
| | H | 18 | 1 × 10⁸ | 2 × 10⁷ | 4 × 10⁶ | + | 9 × 10⁴ | 1 × 10⁴ | 5 × 10⁷ | + | + | − | − | − |
| Vacci- | 3 | 40 | − | − | − | − | − | − | − | − | − | − | − | − |
| nation | 4 | 40 | − | − | − | − | − | − | − | − | − | − | − | − |
| Vacci- | D | 8 | − | 3 × 10²R | 1 × 10²R | +R | 6 × 10²R | 2 × 10²R | − | − | Pasteurella | − | − | − |
| nation & | | (Dyspnea) | | | | | | | | | | | | |
| challenge | E | 18 | 5 × 10⁴ | 8 × 10⁶ | 2 × 10⁶ | + | − | − | − | − | − | − | − | − |
| | F | 18 | 8 × 10⁶ | 3 × 10⁶ | 2 × 10⁵ | + | − | − | − | − | − | − | − | − |
| | 5 | 32 | − | − | − | − | − | − | − | − | − | − | − | − |
| | 6 | 32 | − | − | − | − | − | − | − | − | − | − | − | − |
| | C | 32 | − | − | − | − | − | − | − | − | − | − | − | − |

Day 0: Vaccination - 10¹⁰ cfu/calf P.O.;
Day 14: Challenge infection - 10⁹ cfu/calf P.O.
M.L.N. = Mesenteric lymph node;
A.S. = Abdominal swab;
B.M. = Bone marrow;
C.S.F. = Cerebrospinal fluid;
D.C. = Duodenal contents;
I.C. = Ileal contents;
C.C. = Colic contents;
E = Salmonella were isolated only using the enrichment method;
+ = Positive for Salmonella isolation;
− = Negative for Salmonella isolation;
R = Nalidixic acid and rifampicin resistant strain.

### Example 8

### Salmonella dublin Intramuscular Live Attenuated Vaccine Trials Using a Friesian Calf Model—I

### Objectives

1. To evaluate the safety of N-RM25 vaccine for calves when administered via the intramuscular route.
2. To evaluate the efficacy of intramuscular N-RM25 vaccine to protect calves against salmonellosis caused by S. dublin infection.

### Materials and Methods

### Experimental Animals

For the vaccine trial, ten 2-week-old male Friesian dairy calves were obtained from a herd known to be free of S. dublin infection and divided into 2 groups (see Table 42).

These calves were given colostrum immediately after birth. Prior to obtaining the calves, faeces and sera from each calf were tested bacteriologically and serologically to confirm the calves are free from Salmonella infection. The IgG titre of sera was also measured to investigate for the presence of IgG non-specific to S. dublin that might react to the S. dublin vaccine to produce false positive results in immunological testing.

### Animal Pens

Before challenge infection, one or two calves were placed in each open space pen (5 m×6 m pen surrounded by approximately 1.2 m chain wire fence). After challenge infection with pathogenic bacteria, animals were moved into confined pens in animal solation facilities. The size of each pen is 3.5 m×4.5 m with a 0.7 m×1 m window and a large exhaust fan.

### Capture/Restraint of Animals

For physical examination and sampling, each calf was held by a staff member twice a day (for approximately 15 minutes each time) by gently pushing the animal to the corner of the pen and holding its neck and body.

### Feeding

Milk replacement formula was given to the calves twice a day (8 am and 4 pm) using a feeding bottle or bucket throughout the experiment. Calf pellets were started when the animals were one month old.

### Study Design (see Tables 42 and 43)

1. Calves in the vaccine group were vaccinated via the intramuscular (IM) route on Day 0. These calves were booster vaccinated 13 days post vaccination. The calves were challenge exposed orally 20 days following vaccination. Calves in the control group were only challenge exposed on the same day as calves in vaccine group.
2. Clinical signs were monitored 38 days following vaccination.
3. Faeces, whole blood and serum were taken for bacteriological, haematological, biochemical and serological examination.
4. One calf from the vaccine group was sacrificed on each of Day 6 and Day 18 to investigate the distribution and persistence of the vaccine strain in the organs.
5. The calves were euthanised 38 days post-vaccination or upon showing clinical signs of salmonellosis and subject to full post mortem examination including obtaining tissue specimens for bacteriological and histological examination.

TABLE 42
VACCINATION AND CHALLENGE INFECTION

| | | | | | | Challenge |
|---|---|---|---|---|---|---|
| | Number | | | Booster | | dose* |
| | of | Vaccine | Vaccine dose | vaccine dose | Challenge | (cfu) & |
| Group | animals | strain | (cfu) & route | (cfu) & route | strain | route |
| Vaccine | 6 | S. dublin | 4.1 × 10⁸ IM | 7.2 × 10⁹ IM | S. dublin | 1.8 × 10^{9#} |
| | (Calf A-F) | N-RM25 | in 1 ml PBS | in 3 ml PBS | FD436 | PO |
| Control | 4 | N/A | N/A | N/A | S. dublin | 1.8 × 10^{9#} |
| | (Calf G-J) | | | | FD436 | PO |

Cfu: colony forming unit,
IM: intramuscular,
PBS: phosphate buffered saline, PO: oral.
*The challenge dose was approximately 100 times greater than the 50% infectious dose of FD436.
^{#}To ensure the viability of bacteria after passage through the stomach, the following chemical solution was administered with the bacteria.
1 g MgCO₃, 1 g magnesium trisilicate,
1 g NaHCO₃, 20 ml distilled water.

### Clinical Monitoring

Clinical condition of the calves was monitored twice a day at 9 am and 5 pm. Appetite, body temperature, heart rate, respiration rate, colour of the mucosae, dehydration, faecal consistency, abnormality of eyes and general appearance were monitored at each observation and recorded. When animals showed signs of salmonellosis or other severe symptoms, staff members immediately contacted veterinarians to examine the animals.

### Sampling (Sampling Dates are Indicated in Table 43)

### [Faeces]

Two grams or more of faeces from each calf was collected from the rectum with a sterile cotton swab and transferred into a sterile container. The samples were kept at 5° C. until use.

### [Blood]

An aseptic procedure was used to collect samples (apply 70% ethanol to sterilise sampling sites—usually from jugular vein). Vacuum blood tubes (9 ml) with gel were used to collect blood for IgA and IgG determination. After centrifugation of blood in these tubes at 2700 r.p.m for 10 minutes, sera were collected into small plastic tubes and stored at −70° C. Two-millilitre vacuum tubes with EDTA were used for collecting blood to determine complete blood count and fibrinogen. Three-millilitre disposable syringes with twenty-one-gauge needles were used to collect blood for bacteriological examination.

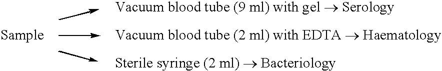

### [Tissue]

Upon autopsy, each tissue sample (approximately 2 cm by 2 cm) for bacteriological studies was aseptically collected and kept at 5° C. until use. For histopathology, tissue samples were fixed with 10% formalin.

### [Content]

Three grams of organ content samples were collected aseptically and kept at 5° C. until use.

The following samples were collected for quantitative and qualitative bacteriological examinations.

### Qualitative Examination:

Lung, liver, spleen, kidney, abomasal content (cont.), duodenal cont., jejunal cont., ileal cont., ileocaecal cont., caecal cont., colonic cont., mesenteric lymph nodes (MLN), bile, cerebrospinal fluid (CSF) and synovial fluid.

### Quantitative Examination:

Lung, liver, spleen, kidney, abomasal cont., duodenal cont., jejunal cont., ileal cont., ileocaecal cont., caecal cont., colonic cont. and bile.

### Bacteriological Examination

### Qualitative Culture for Salmonella

The procedure is as appears in “Salmonella isolation”, “Salmonella identification”, “Identification of nalidixic acid and/or rifampicin resistant strains” and “Polyvalent O antigen slide agglutination test for Salmonella” (in Provisional Specification pages 37, 38 and 39).
The samples were kept at 5° C. for quantitative examinations.

### Quantitative Culture for Salmonella

Each sample was weighed, suspended and homogenised in 5 ml of PBS. The bacterial enumeration method is as appears in “Bacterial enumeration” (in Provisional Specification page 37) was then followed with some modifications. MHA plates containing 0.04% (w/v) nalidixic acid and 0.02% (w/v) rifampicin and XLD plates were used for a viable cell count.
Quantitative culture was performed when faecal, tissue and intestinal content samples were positive for Salmonella using the DI method.
Dry weight was measured for the faeces by keeping it in an incubator room with the lid open until it became dry.

### Enzyme Linked Immunosorbent Assay (ELISA)

Conventional ELISA was performed by the Department of Primary Industries and Fisheries.

### Statistical Analysis

Statistical results were considered significant when probability was less than 5% (P<0.05)).

TABLE 43
INTRAMUSCULAR VACCINE TRIAL TIME TABLE

| Day | Task | Sampling |
|---|---|---|
| −5 | Arrival of calves | |
| −4 | | F |
| −3 | | F |
| −2 | | F |
| −1 | | F |
| 0 | Vaccination | F, BC, S, H |
| 1 | | F, BC |
| 2 | | F, BC, H |
| 3 | | F |
| 4 | | F |
| 5 | | F, BC, H, |
| 6 | Euthanasia and necropsy (Calf E) | |
| 7 | | S |
| 8 | | F, BC |
| 9 | | |
| 10 | | |
| 11 | | F, BC |
| 12 | | |
| 13 | Booster vaccination | F, BC, S, H |
| 14 | | F, BC |
| 15 | | F, BC, H |
| 16 | | F |
| 17 | | F |
| 18 | Euthanasia and necropsy (Calf F) | F, BC, H |
| 19 | | |
| 20 | Challenge infection | F, BC, S, H |
| 21 | | F, BC |
| 22 | | F, BC, H |
| 23 | | F |
| 24 | | F |
| 25 | | F, BC, H |
| 26 | | |
| 27 | | |
| 28 | | F, BC |
| 29 | | |
| 30 | | |
| 31 | | F, BC |
| 32 | | |
| 33 | | |
| 34 | | F, BC |
| 35 | | |
| 36 | | |
| 37 | | F, BC |
| 38 | Euthanasia and necropsy | F, BC, S, H |

F: Faecal sample collection;
BC: Blood culture sample collection;
S: Serum sample collection;
H: Haematology sample collection.

### Results

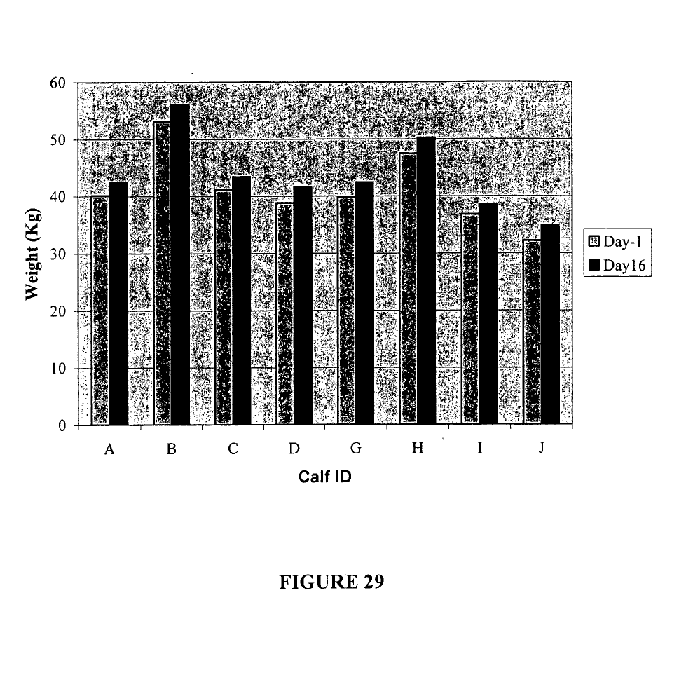

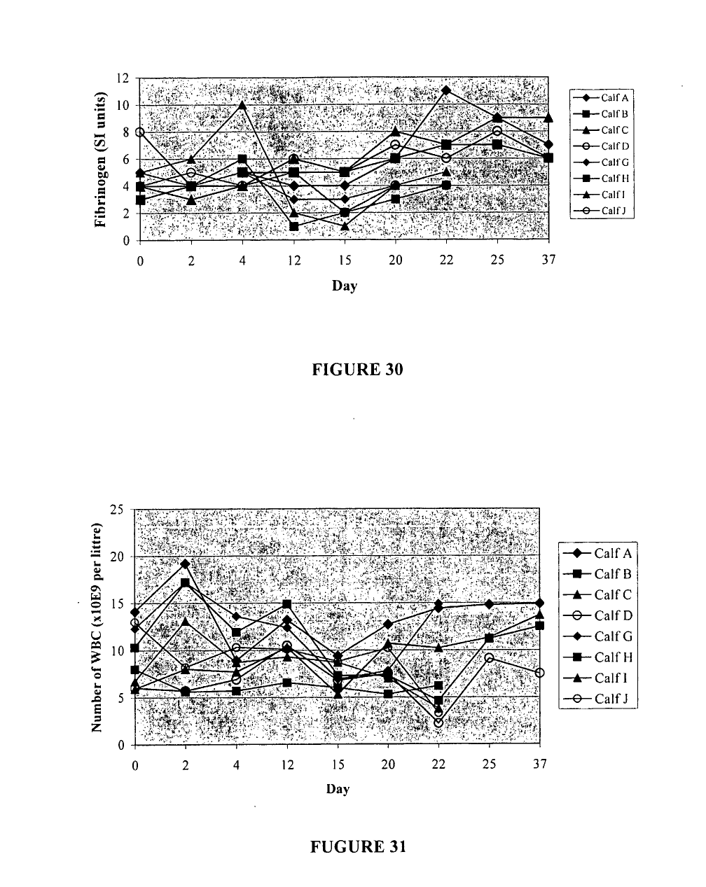

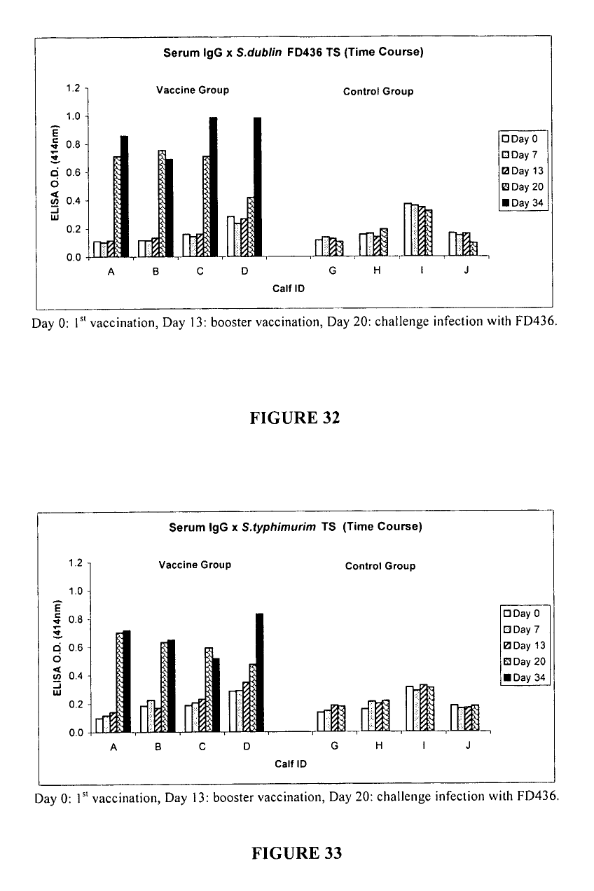

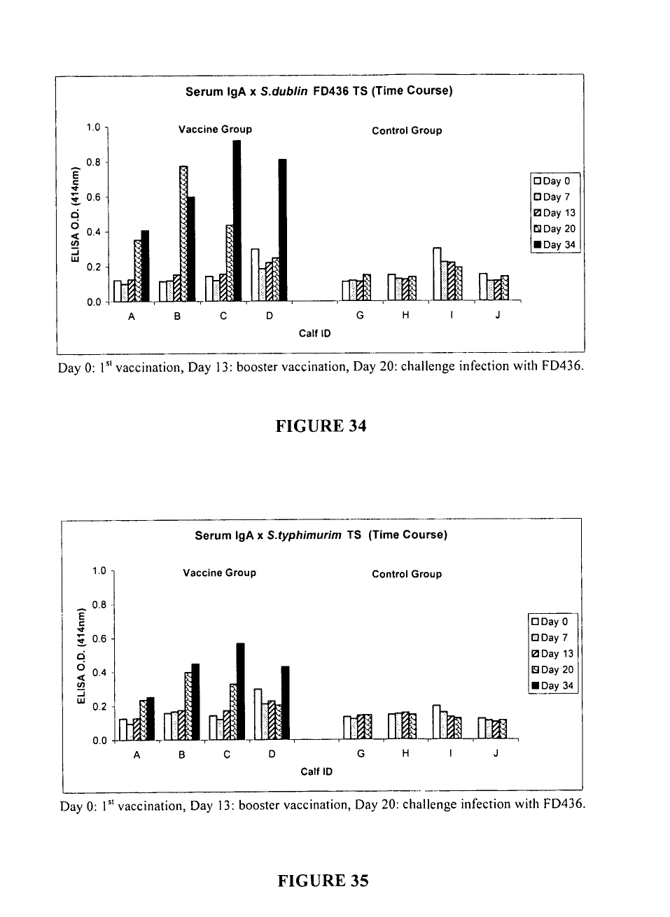

1. No calves produced clinical symptoms following vaccination with N-RM25 (dose: 10⁸ cfu in 1 ml of PBS/animal). All calves showed transient fever and local inflammatory reactions, including swelling, heat and pain, following booster vaccination (10⁹ cfu in 3 ml PBS/animal). Loss of appetite was observed in no calves.
2. There were statistically no significant differences (p>0.05: two-tailed unpaired t test) in increase in body weight between the vaccine and control groups between Day-1 (pre-vaccination) and Day 16 (post-booster vaccination) (see FIG. 29).
3. There were statistically no significant differences in the change in fibrinogen levels (p>0.05: two-tailed unpaired t test) nor in the total white blood cell count (p>0.05: two-tailed unpaired t test) (inflammation indicators) between the vaccine and control groups between Day 0 (pre-vaccination) and Day 12 (post-vaccination but pre-booster vaccination). In contrast, there was a significant difference in the change in fibrinogen levels (p<0.001: two-tailed unpaired t test) between these two groups between Day 12 (pre-booster vaccination) and Day 20 (post-booster vaccination but pre-challenge infection). However, there were no significant differences in both the change in fibrinogen levels (p>0.05: two-tailed unpaired t test) and in the total white blood cell count (p>0.05: two-tailed unpaired t test) between the two groups across these two periods combined (see FIGS. 30 and 31).
4. The vaccine strain (N-RM25) was not isolated from faeces of the calves in either vaccinated or control groups post vaccination (see Table 43).
5. Upon postmortem examinations conducted 6 days and 18 days post-vaccination, only a small number of a nalidixic acid and rifampicin resistant strain (suspected of being the vaccine strain) was isolated from the liver and spleen (see Table 46).
6. All vaccinated calves survived throughout the vaccine trials. In contrast, all control calves were euthanised within 4 days post challenge infection due to severe septicaemic salmonellosis (see Tables 43 and 44).
7. Following lethal challenge infection, all vaccinated calves had a fever (≧40° C.) for up to five days. These calves also produced diarrhoea sometimes containing mucous and blood up to nine days post challenge. Some of them were depressed and showed increased heart rates and respiration rates during this period. However, no vaccinated calves lost appetite. In contrast, all control calves produced significantly more severe symptoms of septicaemic salmonellosis (see Table 45).
8. Following challenge infection, no Salmonella was isolated from blood samples of the vaccinated calves, although the organism was isolated from blood samples of all control calves. All strains isolated from these blood samples were nalidixic acid and rifampicin sensitive (see Table 43).
9. Nalidixic acid and rifampicin sensitive Salmonella was isolated from the faeces of all calves post challenge infection. The vaccinated calves stopped excreting the organism in the faeces within 2 weeks post challenge (see Table 43).
10. Upon postmortem examinations, nalidixic acid and rifampicin sensitive Salmonella was isolated from various organs, tissues and organ contents of the control calves, such as the liver, spleen, lung, kidney, bone marrow, intestinal contents and mesenteric lymph nodes. In contrast, in vaccinated calves, the strain was not isolated from any samples 18 days after challenge infection with the exception of a mesenteric lymph node sample from one calf (see Table 46).
11. There was no significant difference in serum IgG titres on Day 0 between the vaccinated calves and the control calves. In contrast, serum IgG titres of the vaccinated calves against both S. dublin FD436 and S. typhimurium DH436 on Day 20 were significantly (p<0.05) higher than that of the control calves (see FIGS. 32 and 33).
12. There was no significant difference in serum IgA titres on Day 0 between the vaccinated calves and the control calves. In contrast, serum IgA titres of the vaccinated calves against both S. dublin FD436 and S. typhimurium DH436 on Day 20 were significantly (p<0.05) higher than that of the control calves (See FIGS. 34 and 35).

Histopathological examination has yet to be conducted.

### Findings

1. N-RM25 vaccine with a dose of 10⁸ cfu in 1 ml of PBS/animal did not produce any clinical reactions.
2. The vaccine strain was not shed to the environment throughout the vaccine trials.
3. Intramuscular vaccination with N-RM25 protected calves from lethal challenge infection (vaccinated group vs. control group: P<0.05).
4. The challenge strain did not produce bacteremia in the vaccinated calves.
5. The vaccine eliminated the challenge strain from three out of the four calves within 18 days. One calf still had the organism in a mesenteric lymph node but isolation was possible only following enrichment cultivation.
6. There is a high possibility that N-RM25 vaccine produces cross-protection against challenge with S. typhimurium. However, secretory IgA (local immunity in the gut) which may be important to prevent S. typhimurium infection was not determined in the vaccine trials. Secretory IgA reactions following vaccination with N-RM25 are under investigation using a mouse model.

TABLE 44
SALMONELLA ISOLATION FROM FAECES AND BLOOD COLLECTED FROM CALVES IN INTRAMUSCULAR
VACCINE TRIALS WITH N-RM25

| Calf | | | | | | | | | | | | | | | | | | | | | | | | | |
|---|---|---|---|---|---|---|---|---|---|---|---|---|---|---|---|---|---|---|---|---|---|---|---|---|---|
| No. | | Sample | Day 0 | 1 | 2 | 3 | 4 | 5 | 8 | 13 | 14 | 15 | 16 | 17 | 18 | 21 | 22 | 23 | 24 | 25 | 28 | 31 | 34 | 36 | 38 |
| VAC- | E | Faeces | − | − | − | − | − | − | Scheduled euthanasia for autopsy | | | | | | | | | | | | | | | | |
| CINE | | Blood | − | − | − | | | − | | | | | | | | | | | | | | | | | |
| | F | F | − | − | − | − | − | − | − | − | − | − | − | − | − | Scheduled euthanasia for autopsy | | | | | | | | | |
| | | B | − | − | − | | | − | − | − | − | − | − | − | − | | | | | | | | | | |
| | A | F | − | − | − | − | − | − | − | − | − | − | − | − | − | 10⁷ | 10⁸ | +ES | 10⁴ | +ES | +ES | − | − | − | − |
| | | B | − | − | − | | | − | − | − | +R | − | − | − | − | − | − | − | − | − | − | − | − | − | − |
| | B | F | − | − | − | − | − | − | − | − | − | − | − | − | − | 10⁶ | 10⁵ | +ES | − | +ES | − | +ES | − | − | − |
| | | B | − | − | − | | | − | − | − | − | − | − | − | − | − | − | − | − | − | − | − | − | − | − |
| | C | F | − | − | − | − | − | − | − | − | − | − | − | − | − | 10⁷ | 10⁸ | +ES | − | +ES | 10⁴ | − | − | − | − |
| | | B | − | − | − | | | − | − | − | − | − | − | − | − | − | − | − | − | − | − | − | − | − | − |
| | D | F | − | − | − | − | − | − | − | − | − | − | − | − | − | 10⁶ | 10⁷ | 10⁴ | 10⁵ | 10⁵ | 10⁴ | +ES | − | − | − |
| | | B | − | − | − | | | − | − | − | − | − | − | − | − | − | − | − | − | − | − | − | − | − | − |

| CON- | G | F | − | − | − | − | − | − | − | − | − | − | − | − | − | 10⁵ | 10⁸ | Euthanasia due to septicaemic salmonellosis | | |
|---|---|---|---|---|---|---|---|---|---|---|---|---|---|---|---|---|---|---|---|---|
| TROL | | B | − | − | − | | | − | − | − | − | − | − | − | − | +S | +S | | | |
| | I | F | − | − | − | − | − | − | − | − | − | − | − | − | − | 10⁷ | 10⁷ | 10⁸ | Euthanasia due to septicaemic | |
| | | B | − | − | − | | | − | − | − | − | − | − | − | − | − | +S | +S | salmonellosis | |
| | J | F | − | − | − | − | − | − | − | − | − | − | − | − | − | 10⁵ | 10⁶ | 10⁷ | Euthanasia due to septicaemic | |
| | | B | − | − | − | | | − | − | − | − | − | − | − | − | − | +S | +S | salmonellosis | |
| | H | F | − | − | − | − | − | − | − | − | − | − | − | − | − | 10⁶ | 10⁷ | 10⁷ | 10⁶ | Euthanasia due to septicaemic |
| | | B | − | − | − | | | − | − | − | − | − | − | − | − | − | +S | +S | +S | salmonellosis |

Day 0: 1^{st} vaccination,
Day 13 2^{nd} (booster) vaccination,
Day 20: Challenge infection,
+: positive,
−: negative,
E: Salmonella was isolated only following enrichment,
R: nalidixic acid and rifampicin resistant Salmonella,
S: nalidixic acid and rifampicin sensitive Salmonella,
Numbers in the cells indicate the number of Salmonella cells (cfu/g) isolated from faecal samples.

TABLE 45
PROTECTIVE PROPERTIES OF N-RM25 VACCINE ADMINISTERED
VIA INTRAMUSCULAR ROUTE

| | | NO. OF CALVES | |
|---|---|---|---|
| | | EUTHANISED | |
| | NO. OF | DUE | |
| | SURVIVING | TO | PROTECTION¹ |
| GROUP | CALVES | SALMONELLOSIS | (%) |
| VACCINE | 4 | 0 | 100 |
| (N = 4) | | | |
| CONTROL | 0 | 4 | 0 |
| (N = 4) | | | |

¹COMPARISON BETWEEN VACCINE AND CONTROL GROUPS P < 0.05 (TWO-SIDED FISHER'S EXACT TEST)

TABLE 46
TOTAL NUMBER OF CALVES WITH CLINICAL SIGNS MEETING
CRITERIA FOR EUTHANASIA OVER A 10-DAY OBSERVATION
PERIOD FOLLOWING CHALLENGE INFECTION

| | Group | | |
|---|---|---|---|
| | | | p value⁶ |
| | Vaccine | Control¹ | (Vaccine vs. |
| Clinical signs | (n = 4) | (n = 4) | Control) |
| Decrease in appetite | 0/80⁵ | 7/32 | <0.0001 |
| Temperature rise² or drop to | 27/80 | 20/32 | <0.05 |
| lower than usual after peak³ | | | |
| Increase in heart rate⁴ | 11/80 | 10/32 | <0.05 |
| Increase in respiration rate⁴ | 2/80 | 11/32 | <0.0001 |
| Dehydration (>7%) | 0/80 | 0/32 | N/A |
| Lethargy | 8/80 | 15/32 | <0.01 |
| Salmonella isolation from blood | 0/40 | 9/16 | <0.0001 |
| TOTAL | 48/520 | 72/208 | <0.0001 |

Clinical examination was carried out twice a day.
¹Four calves in the control group were euthanised within 4 days post challenge infection due to severe septicaemic salmonellosis.
²Equal to or higher than 40° C.
³More than 1° C. lower than the average pre-challenge (between Day 6 and Day 12) body temperature.
⁴Increase of more than 30% of the average pre-challenge (between Day 6 and Day 12) rate of each calf.
⁵The total number of observations at which calves showed clinical signs over the total number of observations.
⁶Statistical analysis was carried out using two-sided Fisher's exact test.

TABLE 47
SALMONELLA ISOLATION FROM ORGAN, TISSUE AND CONTENT SAMPLES
COLLECTED FROM CALVES IN INTRAMUSCULAR VACCINE TRIALS.

| | Calf | Autopsy | Number of Salmonella in organs, tissues and contents (per gram) | | | | | | |
|---|---|---|---|---|---|---|---|---|---|
| Grp | No. | Date | D.C | J.C. | I.C. | IC.C. | CA.C. | CO.C. | M.L.N |
| Vaccine | E | Day 6 | − | − | − | − | − | − | − |
| | F | 18 | − | − | − | − | − | − | − |
| | A | 38 | − | − | − | − | − | − | +ES |
| | B | 38 | − | − | − | − | − | − | − |
| | C | 38 | − | − | − | − | − | − | − |
| | D | 38 | | − | − | − | − | − | − |
| Control | G | 22 | − | 5.7 × 10²S | 9.5 × 10⁵S | 2.9 × 10⁶S | 5.1 × 10⁶S | 4.1 × 10⁶S | +S |
| | I | 23 | +S | 7.4 × 10⁷S | 2.2 × 10⁹S | 5.6 × 10⁹S | 3.8 × 10⁷S | 4.2 × 10⁸S | +S |
| | J | 23 | − | 2.5 × 10³S | 2.1 × 10⁶S | 1.1 × 10⁶S | 2.8 × 10⁴S | 4.1 × 10⁴S | +S |
| | H | 24 | +S | 1.8 × 10⁸S | 3.9 × 10⁸S | 2.1 × 10⁸S | 2.0 × 10⁸S | 2.7 × 10⁸S | +S |

| | Calf | Autopsy | Number of Salmonella in organs, tissues and contents (per gram) | | | | | | |
|---|---|---|---|---|---|---|---|---|---|
| Grp | No. | Date | Liver | Spleen | Bile | Kidney | Lung | B.M. | C.S.F |
| Vaccine | E | Day 6 | 0.8 × 10R | 2.5 × 10R | − | − | − | − | − |
| | F | 18 | − | +ER | − | − | − | − | − |
| | A | 38 | − | − | − | − | − | − | − |
| | B | 38 | − | − | − | − | − | − | − |
| | C | 38 | − | − | − | − | − | − | − |
| | D | 38 | − | − | − | − | − | | |
| Control | G | 22 | 1.8 × 10⁵S | 3.6 × 10⁵S | − | +ES | 2.1 × 10³S | +ES | − |
| | I | 23 | 2.0 × 10³S | 1.3 × 10³S | − | +ES | 1.7 × 10³S | − | − |
| | J | 23 | 2.3 × 10³S | 1.3 × 10³S | − | +ES | 2.5 × 10³S | − | − |
| | H | 24 | 6.6 × 10⁵S | 1.3 × 10⁶S | − | 4.2 × 10⁴S | 2.9 × 10⁷S | − | − |

Day 0: 1^{st} vaccination,
Day 13: 2^{nd} (booster) vaccination,
Day 20: Challenge infection, −10⁹ cfu/calf P.O with buffer.
D.C.: Duodenal contents;
J.C.: Jejunal contents;
I.C.: Ileal contents;
IC.C.: Ileocaecal contents;
CA.C.: Caecal contents;
CO.C.: Colic contents;
M.L.N.: Mesenteric lymph node;
B.M.: Bone marrow;
C.S.F.: Cerebrospinal fluid;
+: Positive for Salmonella isolation;
−: Negative for Salmonella isolation;
E: Salmonella were isolated only using the enrichment method;
S: Nalidixic and rifampicin sensitive strain;
R: Nalidixic acid and rifampicin resistant strain.

### Example 9

### Salmonella dublin Oral Live Attenuated Vaccine Trials Using a Friesian Calf Model—II

### Objectives

To evaluate the safety of N-RM25 vaccine for calves when administered via the oral route. To evaluate the efficacy of oral N-RM25 vaccine to protect calves against salmonellosis caused by S. dublin infection.

### Materials and Methods

### Experimental Animals

For the vaccine trial, twelve 3 to 4 week old male Friesian calves (Calf No. 1-12) were obtained from a herd known to be free of S. dublin infection and divided into 3 groups (see Table 48). The calves were given colostrum immediately after birth. Prior to the trials, faeces and sera from each calf were tested bacteriologically and serologically to confirm they were free from S. dublin infection.

### Animal Pens

Before challenge infection, one or two calves were placed in each open space pen (5 m×6 m pen surrounded by approximately 1.2 m chain wire fence). After challenge infection with pathogenic bacteria, animals were moved into confined pens in animal isolation facilities. The size of each pen is 3.5 m×4.5 m with a 0.7 m×1m window and a large exhaust fan.

### Capture/Restraint of Animals

For physical examination and sampling, each calf was held by a staff member twice a day (for approximately 15 minutes each time) by gently pushing the animal to the corner of the pen and holding its neck and body.

### Feeding

Milk replacement formula was given to the calves twice a day (8 am and 4 pm) using a feeding bottle or bucket throughout the experiment. Calf pellets were started when the animals were one month old.

### Study Design (see Tables 48 and 49)

1. Calves in the vaccine only and vaccine & challenge groups were vaccinated once via the oral route on Day 0. The calves were challenge exposed orally 14 days following vaccination. Calves in the control group were only challenge exposed on the same day as calves in vaccine group.
2. Clinical signs were monitored for 32 days (for 40 days for the calves in the vaccine group) following vaccination.
3. Faeces, whole blood and serum samples were taken for bacteriological, haematological, biochemical and serological examination.
4. Two calves in the vaccine and challenge group were scheduled for euthanasia 18 days post-vaccination (4 days post-challenge) to compare the distribution and persistence of the vaccine strain in the body between vaccinated and non-vaccinated calves.
5. The calves were euthanised 32 days (40 days for the calves in the vaccine group) post-vaccination or upon showing clinical signs of salmonellosis and subject to full post-mortem examination including obtaining tissue specimens for bacteriological and histological examination.

TABLE 48
VACCINATION AND CHALLENGE INFECTION.

| | Number of | Vaccine | Vaccine dose | Challenge | Challenge dose* |
|---|---|---|---|---|---|
| Group | animals | strain | (cfu) & route | strain | (cfu) & route |
| Vaccine only | 2 | S. dublin | 3.5 × 10¹⁰/calf | N/A | N/A |
| | (Calf 1-2) | N-RM25 | PO^{#} | | |
| Vaccine & | 6 | S. dublin | 3.5 × 10¹⁰/calf | S. dublin | 2.7 × 10⁹/calf PO^{#} |
| Challenge | (Calf 3-8) | N-RM25 | PO^{#} | FD436 | |
| Control | 4 | N/A | N/A | S. dublin | 2.7 × 10⁹/calf PO^{#} |
| | (Calf 9-12) | | | FD436 | |

Cfu: colony forming unit, PO: oral.
*The challenge dose was approximately 100 times greater than the 50% infectious dose of FD436.
^{#}To ensure the viability of bacteria after passage through the stomach, the following chemical solution was administered with the bacteria.
1 g MgCO₃, 1 g magnesium trisilicate,
1 g NaHCO₃, 20 ml distilled water.

### Clinical Monitoring

Clinical condition of the calves was monitored twice a day at 9 am and 5 pm. Appetite, body temperature, heart rate, respiration rate, colour of the mucosae, dehydration, faecal consistency, abnormality of eyes and general appearance were monitored at each observation and recorded. When animals showed signs of salmonellosis or other severe symptoms, staff members immediately contacted veterinarians to examine the animals.

### Sampling (Sampling Dates are Indicated in Table 48)

### [Faeces]

Two grams or more of faeces from each calf was collected from the rectum with a sterile cotton swab and transferred into a sterile container. The samples were kept at 5° C. until use.

### [Blood]

An aseptic procedure was used to collect samples (apply 70% v/v ethanol to sterilise sampling sites—usually from jugular vein). Vacuum blood tubes (9 ml) with gel were used to collect blood for IgA and IgG determination. After centrifugation of blood in these tubes at 2700 r.p.m for 10 minutes, sera were collected into small plastic tubes and stored at −70° C. Two-millilitre vacuum tubes with EDTA were used for collecting blood to determine complete blood count and fibrinogen. Three-millilitre disposable syringes with twenty-one-gauge needles were used to collect blood for bacteriological examination.

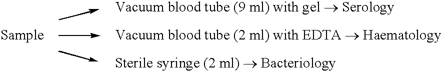

### [Tissue]

Upon autopsy, each tissue sample (approximately 2 cm by 2 cm) for bacteriological studies was aseptically collected and kept at 5° C. until use. For histopathology, tissue samples were fixed with 10% v/v formalin.

### [Content]

Three grams of organ content samples were collected aseptically and kept at 5° C. until use.

The following samples were collected for quantitative and qualitative bacteriological examinations.

### Qualitative Examination:

Lung, liver, spleen, kidney, abomasal content (cont.), duodenal cont., jejunal cont., ileal cont., ileocaecal cont., caecal cont., colonic cont., mesenteric lymph nodes (MLN), bile, cerebrospinal fluid (CSF) and synovial fluid.

### Quantitative Examination:

Lung, liver, spleen, kidney, abomasal cont., duodenal cont., jejunal cont., ileal cont., ileocaecal cont., caecal cont., colonic cont. and bile.

### Bacteriological Examination

### Qualitative Culture for Salmonella

The procedure is as appears in “Salmonella isolation”, “Salmonella identification”, “Identification of nalidixic acid and/or rifampicin resistant strains” and “Polyvalent O antigen slide agglutination test for Salmonella”.
The samples were kept at 5° C. for quantitative examinations.

### Quantitative Culture for Salmonella

Each sample was weighed, suspended and homogenised in 5 ml of PBS. The bacterial enumeration method is as appears in “Bacterial enumeration” was then followed with some modifications. MHA plates containing 0.04% (w/v) nalidixic acid and 0.02% (w/v) rifampicin and XLD plates were used for a viable cell count.
Quantitative culture was performed when faecal, tissue and intestinal content samples were positive for Salmonella using the DI method.
Dry weight was measured for the faeces by keeping it in an incubator room with the lid open until it became dry.

### Statistical Analysis

Statistical results were considered significant when probability was less than 5% (P<0.05)).

TABLE 49
ORAL VACCINE TRIAL TIME TABLE

| Day | Task | Sampling |
|---|---|---|
| −5 | Arrival of calves | |
| −4 | | |
| −3 | | F, S |
| −2 | | |
| −1 | | |
| 0 | Vaccination | F, BC, S, H |
| 1 | | F, BC |
| 2 | | F, BC |
| 3 | | F |
| 4 | | F |
| 5 | | F, BC, H |
| 6 | | |
| 7 | | |
| 8 | | F, BC |
| 9 | | |
| 10 | | |
| 11 | | F, BC |
| 12 | | |
| 13 | | |
| 14 | Challenge infection | F, BC, S, H |
| 15 | | F, BC |
| 16 | | F, BC |
| 17 | | F |
| 18 | Euthanasia and necropsy (Calves 7 and 8) | F, |
| 19 | | F, BC, H |
| 20 | | |
| 21 | | |
| 22 | | F, BC |
| 23 | | |
| 24 | | |
| 25 | | F, BC |
| 26 | | |
| 27 | | |
| 28 | | F, BC, S |
| 29 | | |
| 30 | | |
| 31 | | F, BC |
| 32 | Euthanasia and necropsy (all surviving calves except | |
| | the calves in the vaccine only group) | |
| 33 | | |
| 34 | | F |
| 35 | | |
| 36 | | |
| 37 | | F, BC |
| 38 | | |
| 39 | | |
| 40 | Euthanasia and necropsy (the calves in the vaccine | F |
| | only group) | |

F: Faecal sample;
BC: Blood culture sample;
S: Serum sample;
H: Haematology sample.

### Results (see Tables 50 and 51)

1. No calves produced clinical symptoms, except soft stools, post-vaccination using N-RM25.
2. Nalidixic acid and rifampicin resistant Salmonella (suspected of being the vaccine strain) was isolated from faeces of vaccinated calves using the direct isolation method for 1-2 days post-vaccination. The organism was not isolated from faeces beyond 8 days following vaccination even using the enrichment isolation method.
3. One of the calves in the vaccine and challenge group was euthanised due to severe signs of pneumonia, such as coughing and dyspnea. As Pasteurella multocida was later isolated from the lung, it was determined that the cause of the disease was infection of the lung with this organism.
4. Post-challenge infection, all vaccinated calves had a fever (39.5-40.5° C.) and watery diarrhoea up to 8 days, but they were relatively active and had a stable appetite. Compared with this, calves in the control group produced critical symptoms, such as very high fever (>40.5° C.), depression, anorexia, tachypnea and tachycardia, but only a small amount of diarrhoea.
5. All vaccinated calves, except one which was euthanised due to Pasteurella multocida infection, survived throughout the vaccine trials. In contrast, all control calves were euthanised within 3 days post-challenge infection due to severe septicaemic salmonellosis.
6. Following challenge infection, the number of nalidixic acid and rifampicin sensitive Salmonella (suspected of being the challenge strain) excreted in the faeces of the vaccinated calves was significantly (p<0.05 (two-tailed Student's t test)) fewer than that in the faeces of calves in the control group.
7. Upon autopsy performed between Day 16 and Day 18, nalidixic acid and rifampicin sensitive Salmonella (suspected of being the challenge strain) were isolated from various organs and tissues of the control calves. These organs and tissues include the liver, spleen, intestine, lung, kidney, bile, bone marrow and cerebrospinal fluid. In contrast, the bacteria were isolated only from the intestine and mesenteric lymph node of two calves in the vaccinated and challenge group sacrificed experimentally on Day 18.
8. No Salmonella was isolated from any organs of the vaccinated and challenged calves 32 days post-vaccination (18 days post-challenge infection).
9. No Salmonella was isolated from any organs of the calves in the vaccine only group 40 days post-vaccination (26 days post-challenge infection).
   Histopathological and serological examinations have yet to be conducted.

### Findings

1. Single vaccination with N-RM25 administered via the oral route protected calves from lethal challenge infection (the vaccinated calves vs. the control calves: P<0.01 (two-tailed Fisher's exact test)).
2. The vaccine strain was excreted in the faeces only for a short period (≦8 days).
3. The vaccine strain was eliminated from the animals within 18 days post vaccination.
3. The vaccine eliminated the challenge bacteria from the host within 18 days.

TABLE 50
SALMONELLA ISOLATION FROM FAECES AND BLOOD COLLECTED FROM CALVES IN
VACCINE TRIALS

| | | | | | | | | | | | | | | | |
|---|---|---|---|---|---|---|---|---|---|---|---|---|---|---|---|
| Calf | Sample | Day 0 | 1 | 5 | 6 | 7 | 8 | 9 | 2 | 5 | 8 | 1 | 4 | 7 | 0 |

| | | | | | | | | | | | | | | |
|---|---|---|---|---|---|---|---|---|---|---|---|---|---|---|
| 1 | F | DR | ER | ER | ER | ER | ER | | | | | | | |
| | B | | | | | | | | | | | | | |
| 2 | F | DR | ER | | | | | | | | | | | |
| | B | | | | | | | | | | | | | |
| 3 | F | DR | ER | | ER | | | ×10⁶ | E | E | E | | | Euthanised |
| | B | | | | | | | | | | | | | (no |
| | | | | | | | | | | | | | | symptoms) |
| 4 | F | DR | DR | ER | ER | | | ×10⁶ | ×10⁵ | ×10⁵ | E | E | E | Euthanised |
| | B | | | | | | | | | | | | | (no |
| | | | | | | | | | | | | | | symptoms) |
| 5 | F | DR | ER | ER | ER | | | ×10⁶ | ×10⁵ | E | E | | | Euthanised |
| | B | | | | | | | | | | | | | (no |
| | | | | | | | | | | | | | | symptoms) |

| 6 | F | − | +DR | +ER | +ER | +ER | + | ER | − | Euthanised due to severe signs of pneumonia | | | |
|---|---|---|---|---|---|---|---|---|---|---|---|---|---|
| | B | − | − | − | | | + | − | | (Pateurella multocida was later isolated | | | |
| | | | | | | | | | | from the lung) | | | |
| 7 | F | − | +DR | +ER | − | +ER | +ER | +ER | − | 4 × 10⁶ | 2 × 10⁶ | +E | Euthanised to compare with |
| | B | − | − | − | | | + | − | − | − | − | − | the control calves |
| 8 | F | − | +DR | +ER | − | +ER | +ER | − | − | 1 × 10⁷ | 1 × 10⁶ | +E | Euthanised to compare with |
| | B | − | − | − | | | − | + | − | − | − | − | the control calves |
| 9 | F | − | − | − | +E* | +E* | − | − | − | 3 × 10⁸ | 3 × 10⁸ | Dead due to severe signs of | |
| | B | − | − | − | | | − | − | − | + | + | salmonellosis | |
| 10 | F | − | − | − | − | − | − | − | − | 2 × 10⁷ | 5 × 10⁸ | Euthanised due to severe signs | |
| | B | − | − | − | | | − | − | − | + | + | of salmonellosis | |
| 11 | F | − | − | − | − | − | − | − | − | 9 × 10⁷ | 2 × 10⁶ | 2 × 10⁶ | Euthanised due to severe |
| | B | − | − | − | | | − | − | − | + | + | + | signs of salmonellosis |
| 12 | F | − | − | − | − | − | − | − | − | 7 × 10⁷ | 7 × 10⁷ | 8 × 10⁷ | Euthanised due to |
| | | | | | | | | | | | | | severe signs of |
| | | | | | | | | | | | | | salmonellosis |
| | B | − | − | − | | | − | | − | − | − | + | |

Day 0: vaccination;
Day14: challenge infection;
+: positive;
−: negative;
D: direct isolation method;
E: enrichment isolation method;
R: nalidixic acid and rifampicin resistant strain;
*Salmonella other than S. dublin.
All bacteria strains isolated from the samples post-challenge infection were nalidixic acid and rifampicin sensitive.

TABLE 51
SALMONELLA ISOLATION FROM ORGAN, TISSUE AND CONTENT SAMPLES COLLECTED
FROM CALVES IN VACCINE TRIALS

| | Calf | Autopsy | Organ, tissue and content | | | | | |
|---|---|---|---|---|---|---|---|---|
| Group | No. | Date | D.C. | I.C. | C.C. | M.L.N | Liver | Spleen |
| Control | 10 | 16 | 7 × 10⁵ S | 3 × 10⁷ S | 1 × 10⁷ S | +S | 1 × 10⁴ S | 2 × 10⁵ S |
| | 9 | 17 (Dead) | | | | +S | +S | +S |
| | 11 | 18 | 2 × 10⁴ S | 5 × 10⁴ S | 2 × 10⁵ S | +S | 2 × 10³ S | 2 × 10³ S |
| | 12 | 18 | 1 × 10⁸ S | 2 × 10⁷ S | 4 × 10⁶ S | +S | 9 × 10⁴ S | 1 × 10⁴ S |
| Vaccination & | 6 | 8 (Dyspnea) | − | ×10²R | 1 × 10²R | R | 6 × 10²R | 2 × 10²R |
| challenge | | | | | | | | |
| | 7 | 18 | 5 × 10⁴ S | ×10⁶ S | 2 × 10⁶ S | S | − | − |
| | 8 | 18 | 8 × 10⁶ S | ×10⁶ S | 2 × 10⁵ S | S | − | − |
| | 3 | 32 | − | − | − | | − | − |
| | 4 | 32 | − | − | − | | − | − |
| | 5 | 32 | − | − | − | | − | − |
| Vaccination | 1 | 40 | − | − | − | | − | − |
| only | 2 | 40 | − | − | − | | − | − |

| | Calf | Autopsy | Organ, tissue and content | | | | | |
|---|---|---|---|---|---|---|---|---|
| Group | No. | Date | Bile | Kidney | Lung | A.S. | B.M. | C.S.F. |
| Control | 10 | 16 | − | +S | +S | − | +S | +S |
| | 9 | 17 (Dead) | +S | +S | +S | +S | +S | +S |
| | 11 | 18 | − | +S | − | − | − | − |
| | 12 | 18 | 5 × 10⁷ S | +S | +S | − | − | − |
| Vaccination & | 6 | 8 (Dyspnea) | − | − | Pasteurella | | | |
| challenge | | | | | | | | |
| | 7 | 18 | − | − | − | | | |
| | 8 | 18 | − | − | − | | | |
| | 3 | 32 | − | − | − | | | |
| | 4 | 32 | − | − | − | | | |
| | 5 | 32 | − | − | − | | | |
| Vaccination | 1 | 40 | − | − | − | | | |
| only | 2 | 40 | − | − | − | | | |

Day 0: Vaccination;
Day 14: Challenge infection.
M.L.N.: mesenteric lymph node;
A.S.: abdominal swab;
B.M.: bone marrow;
C.S.F.: cerebrospinal fluid;
D.C.: Duodenal contents;
I.C.: ileal contents;
C.C.: colon contents;
E: Salmonella were isolated only using the enrichment method;
+: positive for Salmonella isolation;
−: negative for Salmonella isolation;
S: nalidixic acid and rifampicin sensitive strain;
R: nalidixic acid and rifampicin resistant strain.

### Example 10

### Studies on Immune Responses in Mice Induced by N-RM25 Administered Via the Parenteral Route

### Objectives

To verify slow “drip feeding” immune activation concept by investigating mucosal immune responses following parenteral inoculation with S. dublin mutant N-RM25 (highly attenuated but remains invasive).

### Major Investigations

1. Penetration of the liver, spleen and gall bladder by N-RM25 and N-RM29 (highly attenuated and has lost invasiveness) following intraperitoneal administration.
2. Penetration of the intestinal lumen and intestine by these strains following intraperitoneal administration.
3. Mucosal immune responses (IgA titre) induced by these strains.
4. Humoral immune responses (IgA and IgG titres) induced by these strains.

### Materials and Methods

### Experimental Animals

Sixty Salmonella free mice (QB out bred, 20-25 g) from the same genetic line were used. The mice were divided into 3 groups (Table 52).

### Feeding

Mice pellets and fresh water were supplied every day.

### Cage Cleaning

Bedding in mice cages were changed everyday. After removing old bedding, the cages were sterilised with 70% v/v ethanol and flaming.

### Study Design (see Tables 52, 53 and 54)

1. N-RM25 and R-NM29 were given to the mice in Groups 2 and 3, respectively. These organisms were administered via the intraperitoneal route.
2. Clinical appearance of the mice (5 parameters: activity, appetite, dehydration, respiratory condition and faecal consistency) was monitored twice a day at 9 am and 4 pm throughout the experiment. Mice showing clinical signs in three or more of the five parameters were classed as being infected by S. dublin and immediately euthanised.
3. Faecal samples (2-3 pellets) for bacteriology from each mouse were collected every morning.
4. At set points in time during the experiments, five mice in each group were fasted for 4 hours, sacrificed and immediately autopsied. Upon autopsy, the heart blood, liver, spleen, gall bladder bile intestinal flash were collected.
5. Heart blood of the sacrificed mice will be investigated for bacteriology and serology (IgA and IgG determination).
6. Gall bladder bile of the mice will be cultivated qualitatively.
7. The livers and spleens of the mice will be investigated for bacteriology and histopathology.
8. Intestinal flash will be used for secretory IgA determination.

TABLE 52
DOSES FOR EXPERIMENTAL INFECTION WITH N-RM25
AND R-NM29.

| | | Number of mice in | Dose |
|---|---|---|---|
| Group | Strain | group | (cfu/mouse)* |
| 1 (control) | — | 20 | 0 |
| 2 | N-RM25 | 20 | 9.8 × 10⁷ |
| 3 | R-NM29 | 20 | 1.1 × 10⁸ |

*Bacteria in 100 μl of PBS were injected in each mouse by the intraperitoneal route. cfu: colony forming unit. Mice in the Group 1 were injected with PBS only.

TABLE 53
TIMETABLE FOR EUTHANASIA.

Day

| Group | 0 | 7 | 14 | 21 |
|---|---|---|---|---|
| 1 | E | E | E | E |
| 2 | E | E | E | E |
| 3 | E | E | E | E |

Day: number of days post-inoculation with S. dublin strains.
E: days on which five mice in each group were euthanised.

### Sampling for Intestinal Flash

The ileum was ligated at a point approximately 15 cm from immediately before the caecum and the intestine cut at the upper side of the ligation and at the end of the ileocaecum to create a section containing the ileum and ileocaecum. The serosal surface of the section was rinsed with sterile cold (4° C.) PBS. A blunt needle (21 G) was inserted into the lumen from the distal end and 2 ml of sterile cold PBS was injected gently into the lumen. The distal end of the section was held tightly to prevent leak of PBS and the section was massaged gently to wash the surface of the intestinal mucosa. The PBS was collected in a small plastic tube and kept at −70° C.

### Euthanasia

Euthanasia was carried out using intraperitoneal injection with Nembutal at a dose of 100 μl/mouse.

TABLE 54
STUDY TIMETABLE

| Day | Task | Sampling |
|---|---|---|
| −3 | | F |
| −2 | | F |
| −1 | | F |
| 0 | Intraperitoneal infection and 1^{st} euthanasia | F, HB, L, S, GB, IF |
| 1 | | F |
| 2 | | F |
| 3 | | F |
| 4 | | F |
| 5 | | F |
| 6 | | F |
| 7 | 2^{nd} euthanasia | F, HB, L, S, GB, IF |
| 8 | | F |
| 9 | | F |
| 10 | | F |
| 11 | | F |
| 12 | | F |
| 13 | | F |
| 14 | 3^{rd} euthanasia | F, HB, L, S, GB, IF |
| 15 | | F |
| 16 | | F |
| 17 | | F |
| 18 | | F |
| 19 | | F |
| 20 | | F |
| 21 | 4^{th} euthanasia | F, HB, L, S, GB, IF |

F: faecal sample,
HB: heart blood sample,
L: liver sample,
S: spleen sample,
GB: gall bladder bile sample,
IF: intestinal flash sample.

### BIBLIOGRAPHY

Ausubel (Ed), Current Protocols in Molecular Biology, 5^{th} Edition, John Wiley & Sons, Inc, NY, 2002.
Black et al., Journal of Infectious Diseases 155:1260-1265, 1987.
Bruner, Cornell Veterinarian 75:93-96, 1985.
Carlton and Brown, Gene Mutation in Manual of Methods for General Bacteriology, pp 232-234, Eds Gerhardt et al., American Society for Microbiology, Washington D.C., USA, 1981.
Collins et al., Microbiological Methods pp 86, 114-115, 178-205, Butterworth-Heineman, Oxford, England, 1995.
Doetsch, Determinative Methods of Light Microscopy In Manual of Methods for General Bacteriology, p 29, Eds Gerhardt, et al., American Society for Microbiology, Washington D.C., USA, 1981.
Ferraro et al., Performance standards for antimicrobial susceptibility testing, NCCCLS 18:1-35, 1998.
Field, Veterinary Journal 104:251-266, 294-302, 323-339, 1948.
Frost and Spradbrow, Veterinary Microbiology I, II, II Practical Manual and Laboratory Notes, p 8, The University of Queensland, Brisbane, Australia.
Gibson, Veterinary Record 73:1284-1295, 1961.
Gibson, Journal of Dairy Research 32:97-134, 1965.
Griggs et al., Antimicrobial Agents and Chemotherapy 40:1009-1013, 1996.
Gomez et al., World Health Statistics Quarterly 50:81-89, 1997.
Hinton, British Veterinary Journal 130:556-562, 1974.
Hitchcock and Brown, Journal of Bacteriology 154:269-277, 1983.
House and Smith, 1997 USAHA Proceedings: evaluation of bovine Salmonella vaccines United Sates Animal Health Association, USA, 1997.
Imberechts, Salmonella serotypes analysed at the VAR in 2000 pp 4-27, Veterinary and Agrochemical Research Centre, Brussels, Belgium, 2001.
Inzana and Apicella, Electrophoresis 20:462-465, 1999.
Jin and Gross, Journal of Molecular Biology 202:45-58, 1988.
Levine et al., Journal of Clinical Investigation 79:888-902, 1987.
Levine et al., Lancet II 79:467-470, 1988.
Lindberg and Robertsson, Infection and Immunity 41:751-757, 1983.
Marolda et al., Journal of Bacteriology 172:3590-3599, 1990.
McClelland et al., Nature 413:852-856, 2001.
Metssalu, A new user's manual, pp 1-27, Labsystems Corporation, Helsinki, Finland, 1995.
Miles et al., Journal of Hygiene Cambridge 38:732-749, 1938
Murray et al., Australian Salmonella Reference Centre Annual Report 2000 p 4, Institute of Medical and Veterinary Science, Adelaide, Australia, 2001.
Reed and Muench, The American Journal of Hygiene 27:493-497, 1937.
Sambrook and Russell, Molecular Cloning: A Laboratory Manual, 3^{rd} Edition, Cold Spring Harbor Laboratory Press, Cold Spring Harbor, N.Y., 2001.
Smibert and Krieg, General Characterization In Manual of Methods for General Bacteriology, p 415, Eds Gerhardt et al., American Society for Microbiology, Washington D.C., USA, 1981.
Smith et al., American Journal of Veterinary Research 45:2231-2235, 1984.
Taylor et al., Journal of Infectious Diseases 146:322-327, 1982.
The Centres for Disease Control and Prevention, MMWR 43:1-7, 1994.
Tsai and Frasch, Analystical biochemistry 119:115-110, 1982.
Vandergraff and Malmo, Australian Veterinary Journal 53:453-455, 1977.
Watts et al., Performance standards for antimicrobial disk and dilution susceptibility test for bacteria isolated from animals, NCCLS 17:1-63, 1994.
World Health Organization, Salmonellosis control: the role of animal and product hygiene pp 7-78, Word Health Organization, Geneva, 1988.
Wray and Sojka, Journal of Dairy Research 44:383-425, 1977.
Wray, Veterinary Record 116:485-489, 1985.
Wray, Irish Veterinary Journal 46:137-140, 1993.

1. A therapeutic agent comprising a microorganism which has a reduced capacity to grow and replicate in the presence of a microbiostatic substance present in, or introduced to, an environment within a subject to which said microorganism migrates following administration wherein said microorganism is capable of inducing an immune response in said subject, which immune response is directed against an antigen on, or secreted by, the microorganism.

2. The therapeutic agent of claim 1 wherein the microbiostatic agent is bile salts.

3. The therapeutic agent of claim 1 or 2 wherein the microorganism is a prokaryote.

4. The therapeutic agent of claim 3 wherein the microorganism is a member of the Enterobacteriaceae.

5. The therapeutic agent of claim 4 wherein the microorganism is a Salmonella sp.

6. The therapeutic agent of claim 5 wherein the microorganism is Salmonella dublin.

7. The therapeutic agent of claim 6 wherein the microorganism comprises an insertion or deletion in an rpoB gene.

8. The therapeutic agent of claim 7 wherein the microorganism is selected from N-RM4, N-RM8, N-RM9, N-RM15, N-RM20, N-RM25, N-RM27 and R-NM29.

9. The therapeutic agent of claim 1 wherein the subject is a mammal.

10. The therapeutic agent of claim 9 wherein the subject is a livestock animal.

11. The therapeutic agent of claim 10 wherein the livestock animal is selected from the list consisting of a cow, a sheep and a pig.

12. The therapeutic agent of claim 9 wherein the mammal is a laboratory test animal.

13. The therapeutic agent of claim 12 wherein the laboratory test animal is selected from the list consisting of a mouse, a rat, a rabbit and a guinea pig.

14. The therapeutic agent of claim 9 wherein the subject is a human.

15. The therapeutic agent of claim 1 wherein the microorganism is rendered unable to grow or replicate in the presence of the microbiostatic substance by exposing the microorganism to nalidisic acid and rifampicin or chemical or functional equivalents thereof for a time and under conditions sufficient to induce a metabolic-drift mutation.

16. The therapeutic agent of claim 1 wherein the antigen is naturally occurring with said microorganism.

17. The therapeutic agent of claim 1 wherein the antigen is introduced to said microorganism.

18. The therapeutic agent of claim 1 wherein the microorganism induces a humoral and/or T-cell-mediated immune response.

19. The therapeutic agent of claim 16 wherein the microorganism induces a mucosal immune response.

20. A therapeutic agent comprising a Salmonella sp. which carries a metabolic-drift mutation resulting in a reduced capacity to grow and replicate in the presence of bile salts present in a subject to which the therapeutic agent is administered said Salmonella sp. is capable of inducing an immune response against itself or an antigen produced by itself.

21. The therapeutic agent of claim 20 wherein the Salmonella sp. is Salmonella dublin.

22. The therapeutic agent of claim 21 wherein the microorganism comprises an insertion or deletion in an rpoB gene.

23. The therapeutic agent of claim 22 wherein the microorganism is selected from N-RM4, N-RM8, N-RM9, N-RM15, N-RM20, N-RM25, N-RM27 and R-NM29.

24. The therapeutic agent of claim 21 wherein the subject is a livestock animal.

25. The therapeutic agent of claim 24 wherein the subject is a cow, a sheep or a pig.

26. The therapeutic agent of claim 25 wherein the subject is a cow.

27. The therapeutic agent of claim 20 wherein the immune response is a humoral immune response.

28. The therapeutic agent of claim 27 wherein the humoral immune response is a mucosal immune response.

29. A method of vaccinating a subject against a microorganism or an antigen produced by a microorganism said method comprising selecting a microorganism, exposing the microorganism to naladixic acid and rifampicin or their chemical or functional equivalents for a time and under conditions sufficient to induce a metabolic drift mutation which renders the microorganism substantially unable to grow or replicate in the presence of a selected microbiostatic agent, and administering said mutated microorganism to the subject under conditions sufficient for the microorganism to migrate to an environment comprising the microbiostatic agent where it maintains itself for a time sufficient for an immune response to be induced to the microorganism or an antigen produced thereby.

30. The method of claim 29 wherein the microbiostatic agent is bile salts.

31. The method of claim 29 or 30 wherein the microorganism is a prokaryote.

32. The method of claim 31 wherein the microorganism is a member of the Enterobacteriaceae.

33. The method of claim 32 wherein the microorganism is a Salmonella sp.

34. The method of claim 33 wherein the microorganism is Salmonella dublin.

35. The therapeutic agent of claim 34 wherein the microorganism comprises an insertion or deletion in an rpoB gene.

36. The therapeutic agent of claim 35 wherein the microorganism is selected from N-RM4, N-RM8, N-RM9, N-RM15, N-RM20, N-RM25, N-RM27 and R-NM29.

37. The method of claim 29 wherein the subject is a mammal.

38. The method of claim 37 wherein the subject is a livestock animal.

39. The method of claim 38 wherein the livestock animal is selected from a cow, a sheep and a pig.

40. The method of claim 37 wherein the mammal is a laboratory test animal.

41. The method of claim 40 wherein the laboratory test animal is selected from the list consisting of a mouse, a rat, a rabbit and a guinea pig.

42. The method of claim 37 wherein the mammal is a human.

43. The method of claim 41 wherein the microorganism induces a humoral and/or a T-cell-mediated immune response.

44. The method of claim 43 wherein the microorganism induces a mucosal immune response.

45. A purified culture of a Salmonella sp. comprising cells which have been exposed to naladixic acid and rifampicin or their chemical or functional equivalents under conditions sufficient to induce a metabolic-drift mutation which renders the Salmonella sp. substantially incapable of growing or replicating in the presence of bile salts.

46. The purified culture of claim 45 wherein the Salmonella sp. is Salmonella dublin.

47. The purified culture of claim 45 or 46 wherein the culture is freeze dried, frozen or reconstituted.

48. Use of the purified culture of claim 45 or 46 or 42 in the manufacture of a vaccine to induce an immune response in a mammal to a Salmonella sp.


SEQUENCE LISTING (6 sequences)

SEQ ID NO: 1 - DNA, 20 bp, salmonella
tgtccgagatggcctgaagc

SEQ ID NO: 2 - DNA, 20 bp, salmonella
taccgtcatagttatccacg

SEQ ID NO: 3 - DNA, 20 bp, salmonella
tcggcaaccgtcgtatccgt

SEQ ID NO: 4 - DNA, 20 bp, salmonella
tcgcacccatcaacgcacgg

SEQ ID NO: 5 - DNA, 12 bp, salmonella
tctcagtttatg

SEQ ID NO: 6 - DNA, 6 bp, salmonella
gaccag